(19) United States
(12) Patent Application Publication
(10) Pub. No.: US 2021/0269451 A1
(43) Pub. Date: Sep. 2, 2021

(54) PYRIDINYL HETEROCYCLYL COMPOUNDS FOR THE TREATMENT OF AUTOIMMUNE DISEASE

(71) Applicant: Hoffmann-La Roche Inc., Little Falls, NJ (US)

(72) Inventors: HAIXIA LIU, SHANGHAI (CN); HONG SHEN, SHANGHAI (CN); WEI ZHU, SHANGHAI (CN); TAISHAN HU, SHANGHAI (CN); ZHISEN ZHANG, SHANGHAI (CN); ZHIWEI ZHANG, SHANGHAI (CN); FABIAN DEY, Basel (CH); XIAOQING WANG, SHANGHAI (CN)

(73) Assignee: Hoffmann-La Roche Inc., Little Falls, NJ (US)

(21) Appl. No.: 17/251,490

(22) PCT Filed: Jun. 11, 2019
(86) PCT No.: PCT/EP2019/065121

§ 371 Date: Dec. 11, 2020

(30) Foreign Application Priority Data
Jun. 13, 2018 (CN) PCT/CN2018/091078

(51) Int. Cl. C07D 487/10; C07D 498/10; C07D 413/14; C07D 487/04; C07D 498/04; C07D 471/04

(52) U.S. Cl. CPC C07D 487/10; C07D 498/10; C07D 471/04; C07D 487/04; C07D 498/04; C07D 413/14

(57) ABSTRACT

The present invention relates to compounds of formula (I), a b (I), wherein R¹ to R⁴ and L are as described herein, and their pharmaceutically acceptable salt, enantiomer or diastereomer thereof, and compositions including the compounds and methods of using the compounds.

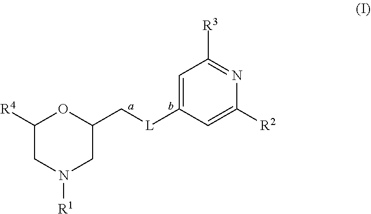

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SLE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721). Other biologics, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll Like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7,8,9 is considered as a key node in a broad of autoimmune and auto-inflammatory diseases (Krieg, A. M. et al. Immunol. Rev. 2007, 220, 251. Jimenez-Dalmaroni, M. J. et al Autoimmun Rev. 2016, 15, 1. Chen, J. Q., et al. Clinical Reviews in Allergy & Immunology 2016, 50, 1). Anti-RNA and anti-DNA antibodies are well established diagnostic markers of SLE, and these antibodies can deliver both self-RNA and self-DNA to endosomes. While self-RNA complexes can be recognized by TLR7 and TLR8, self-DNA complexes can trigger TLR9 activation. Indeed, defective clearance of self-RNA and self-DNA from blood and/or tissues is evident in SLE (Systemic Lupus Erythematosus) patients. TLR7 and TLR9 have been reported to be upregulated in SLE tissues, and correlate with chronicity and activity of lupus nephritis, respectively. In B cells of SLE patients, TLR7 expression correlates with anti-RNP antibody production, while TLR9 expression with IL-6 and anti-dsDNA antibody levels. Consistently, in lupus mouse models, TLR7 is required for anti-RNA antibodies, and TLR9 is required for anti-nucleosome antibody. On the other hand, overexpression of TLR7 or human TLR8 in mice promotes autoimmunity and autoinflammation. Moreover, activation of TLR8 specifically contributes to inflammatory cytokine secretion of mDC/macrophages, neutrophil NETosis, induction of Th17 cells, and suppression of Treg cells. In addition to the described role of TLR9 in promoting autoantibody production of B cells, activation of TLR9 by self-DNA in pDC also leads to induction of type I IFNs and other inflammatory cytokines. Given these roles of TLR9 in both pDC and B cells, both as key contributors to the pathogenesis of autoimmune diseases, and the extensive presence of self-DNA complexes that could readily activate TLR9 in many patients with autoimmune diseases, it may have extra benefit to further block self-DNA mediated TLR9 pathways on top of inhibition of TLR7 and TLR8 pathways. Taken together, TLR7, 8, and 9 pathways represent new therapeutic targets for the treatment of autoimmune and auto-inflammatory diseases, for which no effective steroid-free and non-cytotoxic oral drugs exist, and inhibition of all these pathways from the very upstream may deliver satisfying therapeutic effects. As such, we invented oral compounds that target and suppress TLR7, TLR8 and TLR9 for the treatment of autoimmune and auto-inflammatory diseases.

### SUMMARY OF THE INVENTION

The present invention relates to novel compounds of formula (I) or (Ia),

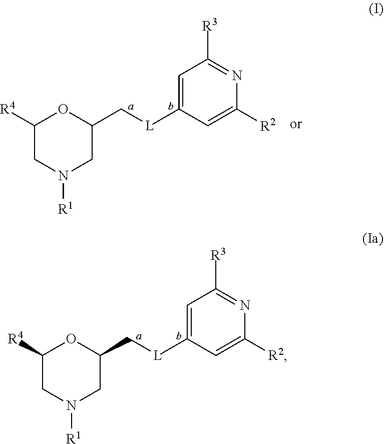

wherein
R¹ is

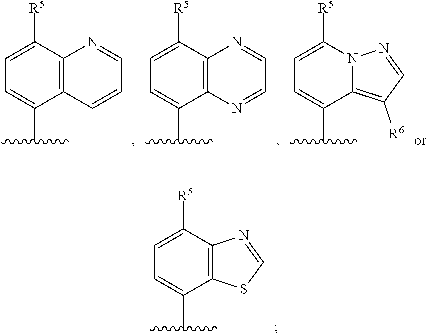

wherein R⁵ is cyano or halogen; R⁶ is H or halogen;
R² is H, amino or C₁₋₆alkyl;
R³ is amino, C₁₋₆alkylamino, C₁₋₆alkyl, haloC₁₋₆alkyl, heterocyclyl, hydroxyC₁₋₆alkyl or C₃₋₇cycloalkyl;
R⁴ is C₁₋₆alkyl;
L is 1,3,3a,4,6,6a-hexahydropyrrolo[3,4-c]pyrrolyl; 1,6-diazaspiro[3.3]heptanyl; 2,3,4a,5,7,7a-hexahydropyrrolo[3,4-b][1,4]oxazinyl; 2,6-diazaspiro[3.3]heptanyl; 2,7-diazaspiro[3.4]octanyl; 5-oxa-2,8-diazaspiro[3.5]nonanyl; (C₁₋₆alkyl)aminoazetidinyl; aminoazetidinyl; azetidinyl(C₁₋₆alkyl)amino; azetidinylamino; (phenylC₁₋₆alkyl)piperazinyl; (hydroxyC₁₋₆alkyl)piperazinyl; (C₁₋₆alkyl)piperazinyl; piperazinyl; piperidinyl; (C₁₋₆alkyl)aminopiperidinyl; aminohalopiperidinyl; amino(hydroxy)piperidinyl; aminopiperidinyl; piperidinylamino; amino(hydroxy)pyrrolidinyl; aminopyrrolidinyl; or pyrrolidinylamino;
or a pharmaceutically acceptable salt, enantiomer or diastereomer thereof.

Another object of the present invention is related to novel compounds of formula (I) or (Ia), their manufacture, medicaments based on a compound in accordance with the invention and their production as well as the use of compounds of formula (I) or (Ia) as TLR7 and/or TLR8 and/or TLR9 antagonist, and for the treatment or prophylaxis of systemic lupus erythematosus or lupus nephritis. The compounds of formula (I) or (Ia) show superior TLR7 and/or TLR8 and/or TLR9 antagonism activity. In addition, the compounds of formula (I) or (Ia) also show good cytotoxicity, solubility, hPBMC, human microsome stability and SDPK profiles, as well as low CYP inhibition.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl, trifluoromethyl and trifluoroethyl.

The term “C₃₋₇cycloalkyl” denotes a saturated monocyclic or bicyclic carbon ring containing from 3 to 7 carbon atoms, particularly from 3 to 6 carbon atoms, for example, cyclopropyl, cyclobutyl, cyclopentyl, cyclohexyl, cycloheptyl, bicyclo[1.1.1]pentanyl and the like. Particular “C₃₋₇cycloalkyl” group is cyclopropyl.

The term “heterocyclyl” denotes a monovalent saturated or partly unsaturated mono-, bicyclic or tricyclic ring system of 3 to 12 ring atoms, comprising 1, 2, or 3 ring heteroatoms selected from N, O and S, the remaining ring atoms being carbon. In particular embodiments, heterocyclyl is a monovalent saturated or partly unsaturated monocyclic or bicyclic ring system of 4 to 10 ring atoms, comprising 1, 2, or 3 ring heteroatoms selected from N, O and S, the remaining ring atoms being carbon. Examples for monocyclic saturated heterocyclyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Bicyclic heterocyclyl can be spiro ring, fused ring or bridged ring. Examples for partly unsaturated heterocyclyl are dihydrofuryl, imidazolinyl, dihydro-oxazolyl, tetrahydro-pyridinyl, or dihydropyranyl. Monocyclic or bicyclic or tricyclic heterocyclyl can be further substituted once, twice or three times by hydroxyC₁₋₆alkyl, amino, aminoC₁₋₆alkyl, C₁₋₆alkoxy, C₁₋₆alkoxyC₁₋₆alkyl, C₁₋₆alkyl, (C₁₋₆alkyl)₂amino, C₃₋₇ cyclo alkyl, C₃₋₇ cycloalkylamino, haloC₁₋₆alkyl, halogen, hydroxy, hydroxyC₁₋₆alkyl or pyrrolidinyl.

The term “enantiomer” denotes two stereoisomers of a compound which are non-superimposable mirror images of one another.

The term “diastereomer” denotes a stereoisomer with two or more centers of chirality and whose molecules are not mirror images of one another. Diastereomers have different physical properties, e.g. melting points, boiling points, spectral properties, and reactivities.

The term “cis-isomers” and “trans-isomers” denote the relative stereochemistry of the molecule or moiety. For example: compound 10b

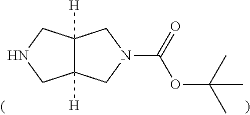

as the “cis-isomers” refers to a mixture of

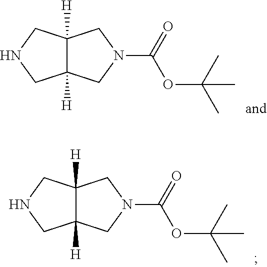

similarly, compound 26a

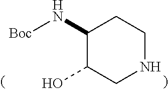 [SMILES: CC(C)(C)OC(=O)N[C@H]1CCNC[C@@H]1O]

as the “trans-isomers” refers to a mixture of

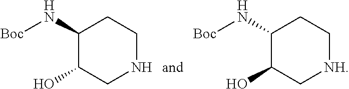 [SMILES: CC(C)(C)OC(=O)N[C@H]1CCNC[C@@H]1O ; CC(C)(C)OC(=O)N[C@@H]1CCNC[C@H]1O]

The way of showing relative stereochemistry also applies to the final compounds.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Antagonist of TLR7 and/or TLR8 and/or TLR9

The present invention relates to a compound of formula (I),

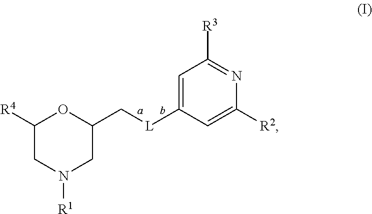

wherein
R¹ is

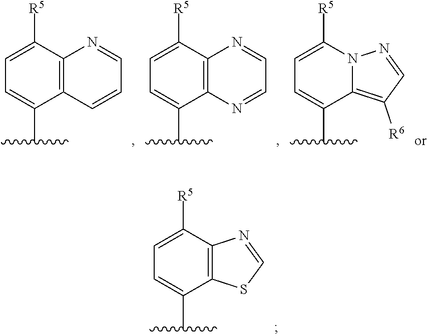

wherein R⁵ is cyano or halogen; R⁶ is H or halogen;
R² is H, amino or C₁₋₆alkyl;
R³ is amino, C₁₋₆alkylamino, C₁₋₆alkyl, haloC₁₋₆alkyl, heterocyclyl, hydroxyC₁₋₆alkyl or C₃₋₇cycloalkyl;
R⁴ is C₁₋₆alkyl;
L is 1,3,3a,4,6,6a-hexahydropyrrolo[3,4-c]pyrrolyl; 1,6-diazaspiro[3.3]heptanyl; 2,3,4a,5,7,7a-hexahydropyrrolo[3,4-b][1,4]oxazinyl; 2,6-diazaspiro[3.3]heptanyl; 2,7-diazaspiro[3.4]octanyl; 5-oxa-2,8-diazaspiro[3.5]nonanyl; (C₁₋₆alkyl)aminoazetidinyl; aminoazetidinyl; azetidinyl(C₁₋₆alkyl)amino; azetidinylamino; (phenylC₁₋₆alkyl)piperazinyl; (hydroxyC₁₋₆alkyl)piperazinyl; (C₁₋₆alkyl)piperazinyl; piperazinyl; piperidinyl; (C₁₋₆alkyl)aminopiperidinyl; aminohalopiperidinyl; amino(hydroxy)piperidinyl; aminopiperidinyl; piperidinylamino; amino(hydroxy)pyrrolidinyl; aminopyrrolidinyl; or pyrrolidinylamino;

or a pharmaceutically acceptable salt, enantiomer or diastereomer thereof.

A another embodiment of present invention is (ii) a compound of formula (Ia),

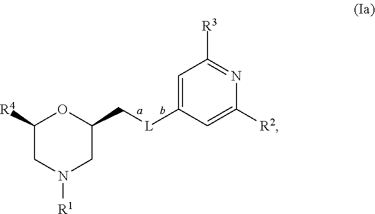

wherein
R¹ is

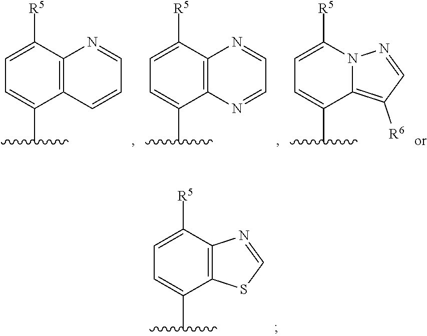

wherein R⁵ is cyano or halogen; R⁶ is H or halogen;
R² is H, amino or C₁₋₆alkyl;
R³ is amino, C₁₋₆alkylamino, C₁₋₆alkyl, haloC₁₋₆alkyl, heterocyclyl, hydroxyC₁₋₆alkyl or C₃₋₇cycloalkyl;
R⁴ is C₁₋₆alkyl;
L is 1,3,3a,4,6,6a-hexahydropyrrolo[3,4-c]pyrrolyl; 1,6-diazaspiro[3.3]heptanyl; 2,3,4a,5,7,7a-hexahydropyrrolo[3,4-b][1,4]oxazinyl; 2,6-diazaspiro[3.3]heptanyl; 2,7-diazaspiro[3.4]octanyl; 5-oxa-2,8-diazaspiro[3.5]nonanyl; (C₁₋₆alkyl)aminoazetidinyl; aminoazetidinyl; azetidinyl(C₁₋₆alkyl)amino; azetidinylamino; (phenylC₁₋₆alkyl)piperazinyl; (hydroxyC₁₋₆alkyl)piperazinyl; (C₁₋₆alkyl)piperazinyl; piperazinyl; piperidinyl; (C₁₋₆alkyl)aminopiperidinyl; aminohalopiperidinyl; amino(hydroxy)piperidinyl; aminopiperidinyl; piperidinylamino; amino(hydroxy)pyrrolidinyl; aminopyrrolidinyl; or pyrrolidinylamino;
   or a pharmaceutically acceptable salt, enantiomer or diastereomer thereof.

A further embodiment of present invention is (iii) a compound of formula (I) or (Ia) according to (i) or (ii), wherein
R¹ is

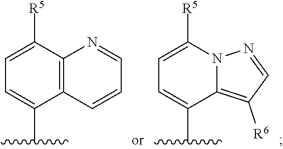

wherein R⁵ is cyano, R⁶ is H.

A further embodiment of present invention is (iv) a compound of formula (I) or (Ia) according to (iii), wherein L is

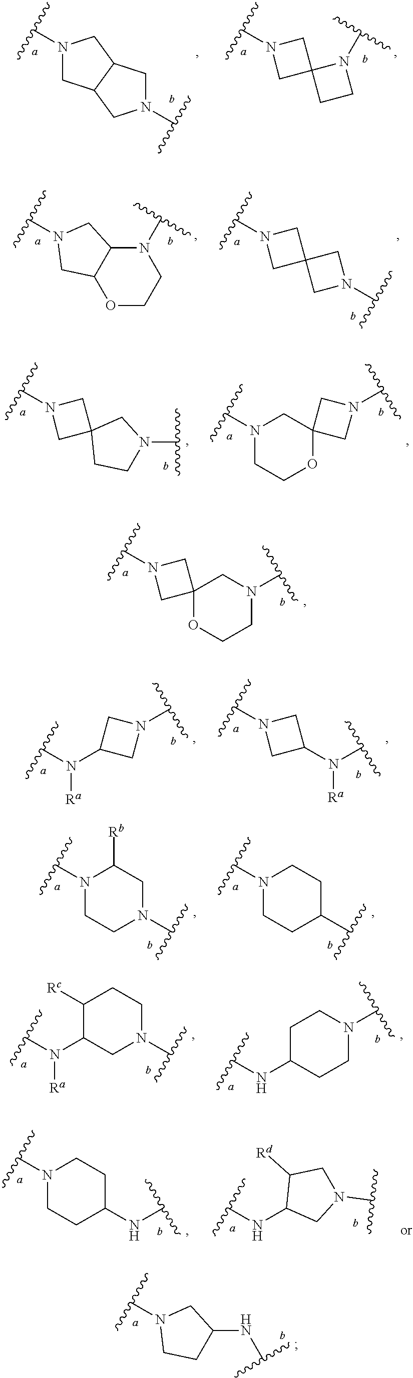

wherein R^{a} is H or C₁₋₆alkyl; R^{b} is H, phenylC₁₋₆alkyl, hydroxyC₁₋₆alkyl or C₁₋₆alkyl; R^{c} is H, halogen or hydroxy; R^{d} is H or hydroxy.

A further embodiment of present invention is (v) a compound of formula (I) or (Ia) according to (iv), wherein L is

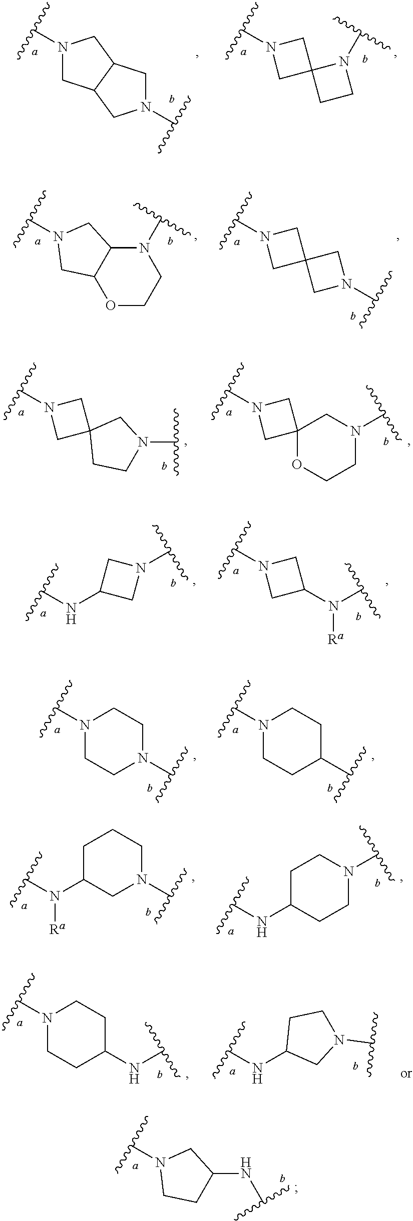

wherein R^{a} is H or C₁₋₆alkyl.

A further embodiment of present invention is (vi) a compound of formula (I) or (Ia) according to (iv), wherein L is

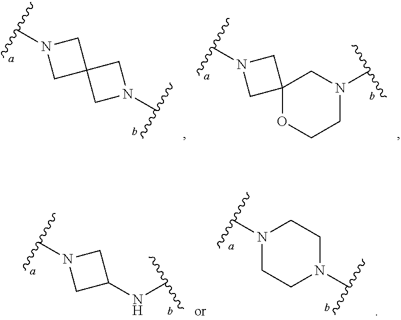 [SMILES: *N1CC2(C1)CN(*)C2 ; *N1CCOC2(C1)CN(*)C2 ; *NC1CN(*)C1 ; *N1CCN(*)CC1]

A further embodiment of present invention is (vii) a compound of formula (I) or (Ia) according to (vi), wherein R³ is amino, C₁₋₆alkylamino, C₁₋₆alkyl, hydroxyC₁₋₆alkyl or C₃₋₇cycloalkyl.

A further embodiment of present invention is (viii) a compound of formula (I) or (Ia) according to (vii), wherein R³ is amino, cyclopropyl, hydroxyethyl, hydroxymethyl, methyl or methylamino.

A further embodiment of present invention is (ix) a compound of formula (I) or (Ia) according to (i) or (ii), wherein
R¹ is

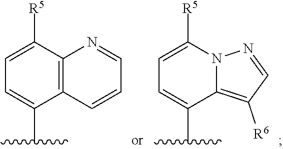

wherein R⁵ is cyano; R⁶ is H;
R² is H or C₁₋₆alkyl;
R³ is C₁₋₆alkyl, hydroxyC₁₋₆alkyl or C₃₋₇cyclo alkyl;
R⁴ is C₁₋₆alkyl;

### L is

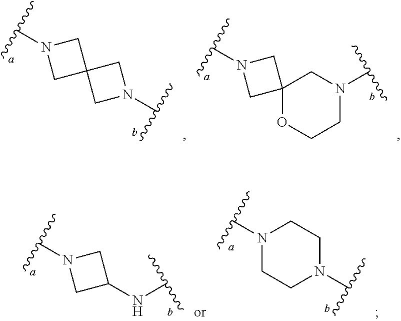 [SMILES: *N1CC2(C1)CN(*)C2 ; *N1CCOC2(C1)CN(*)C2 ; *NC1CN(*)C1 ; *N1CCN(*)CC1]

or a pharmaceutically acceptable salt, enantiomer or diastereomer thereof.

A further embodiment of present invention is (x) a compound of formula (I) or (Ia) according to (ix), wherein
R¹ is

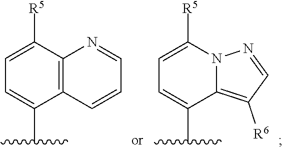

wherein R⁵ is cyano; R⁶ is H;
R² is H or methyl;
R³ is methyl, hydroxymethyl or cyclopropyl;
R⁴ is methyl;
L is

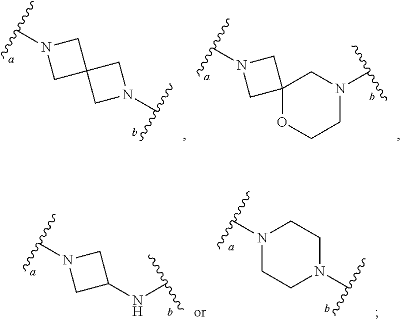 [SMILES: *N1CC2(C1)CN(*)C2 ; *N1CCOC2(C1)CN(*)C2 ; *NC1CN(*)C1 ; *N1CCN(*)CC1]

or a pharmaceutically acceptable salt, enantiomer or diastereomer thereof.

Another embodiment of present invention is that (xi) a compound of formula (I) or (Ia) selected from the following:

5-[(2S,6R)-2-[[2-[2-(hydroxymethyl)-6-methyl-4-pyridyl]-2,6-diazaspiro[3.3]heptan-6-yl]methyl]-6-methyl-morpholin-4-yl]quinoline-8-carbonitrile;

5-[(2S,6R)-2-[[8-(2-cyclopropyl-4-pyridyl)-5-oxa-2,8-diazaspiro[3.5]nonan-2-yl]methyl]-6-methyl-morpholin-4-yl]quinoline-8-carbonitrile;

5-[(2S,6R)-2-[[4-[2-(hydroxymethyl)-6-methyl-4-pyridyl]-2-methyl-piperazin-1-yl]methyl]-6-methyl-morpholin-4-yl]quinoline-8-carbonitrile;

5-[(2S,6R)-2-[[(2S)-4-[2-(hydroxymethyl)-6-methyl-4-pyridyl]-2-methyl-piperazin-1-yl]methyl]-6-methyl-morpholin-4-yl]quinoline-8-carbonitrile;

5-[(2S,6R)-2-[[(2R)-4-[2-(hydroxymethyl)-6-methyl-4-pyridyl]-2-methyl-piperazin-1-yl]methyl]-6-methyl-morpholin-4-yl]quinoline-8-carbonitrile;

5-[(2S,6R)-2-[[4-[[2-(hydroxymethyl)-6-methyl-4-pyridyl]amino]-1-piperidyl]methyl]-6-methyl-morpholin-4-yl]quinoline-8-carbonitrile;

5-[(2S,6R)-2-[[3-[[2-(hydroxymethyl)-6-methyl-4-pyridyl]amino]pyrrolidin-1-yl]methyl]-6-methyl-morpholin-4-yl]quinoline-8-carbonitrile;

5-[(2S,6R)-2-[[3-[(2,6-dimethyl-4-pyridyl)amino]azetidin-1-yl]methyl]-6-methyl-morpholin-4-yl]quinoline-8-carbonitrile;

5-[(2S,6R)-2-[[1-(2,6-dimethyl-4-pyridyl)-1,6-diazaspiro[3.3]heptan-6-yl]methyl]-6-methyl-morpholin-4-yl]quinoline-8-carbonitrile;

5-[(2S,6R)-2-[[7-(2,6-dimethyl-4-pyridyl)-2,7-diazaspiro[3.4]octan-2-yl]methyl]-6-methyl-morpholin-4-yl]quinoline-8-carbonitrile;

cis-5-[(2S,6R)-2-[[5-[2-(hydroxymethyl)-6-methyl-4-pyridyl]-1,3,3a,4,6,6a-hexahydropyrrolo[3,4-c]pyrrol-2-yl]methyl]-6-methyl-morpholin-4-yl]quinoline-8-carbonitrile;

5-[(2S,6R)-2-[[3-[(2,6-dimethyl-4-pyridyl)-methyl-amino]azetidin-1-yl]methyl]-6-methyl-morpholin-4-yl]quinoline-8-carbonitrile;

5-[(2S,6R)-2-[[1-[2-(hydroxymethyl)-6-methyl-4-pyridyl]-1,6-diazaspiro[3.3]heptan-6-yl]methyl]-6-methyl-morpholin-4-yl]quinoline-8-carbonitrile;

5-[(2S,6R)-2-[[2-(2,6-dimethyl-4-pyridyl)-5-oxa-2,8-diazaspiro[3.5]nonan-8-yl]methyl]-6-methyl-morpholin-4-yl]quinoline-8-carbonitrile;

5-[(2S,6R)-2-[[8-(2,6-dimethyl-4-pyridyl)-5-oxa-2,8-diazaspiro[3.5]nonan-2-yl]methyl]-6-methyl-morpholin-4-yl]quinoline-8-carbonitrile;

5-[(2R,6S)-2-methyl-6-[[1-(2-methyl-4-pyridyl)-1,6-diazaspiro[3.3]heptan-6-yl]methyl]morpholin-4-yl]quinoline-8-carbonitrile;

5-[(2R,6S)-2-methyl-6-[[8-(2-methyl-4-pyridyl)-5-oxa-2,8-diazaspiro[3.5]nonan-2-yl]methyl]morpholin-4-yl]quinoline-8-carbonitrile;

5-[(2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile;

trans-5-[(2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile;

cis-5-[(2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile;

5-[(2S,6R)-2-[[4-(2-amino-4-pyridyl)piperazin-1-yl]methyl]-6-methyl-morpholin-4-yl]quinoline-8-carbonitrile;

5-[(2S,6R)-2-[[[1-(2-amino-4-pyridyl)-4-piperidyl]amino]methyl]-6-methyl-morpholin-4-yl]quinoline-8-carbonitrile;

5-[(2S,6R)-2-[[[(3R)-1-(2-amino-4-pyridyl)-3-piperidyl]amino]methyl]-6-methyl-morpholin-4-yl]quinoline-8-carbonitrile;

5-[(2S,6R)-2-[[[(3S)-1-(2-amino-4-pyridyl)-3-piperidyl]amino]methyl]-6-methyl-morpholin-4-yl]quinoline-8-carbonitrile;

5-[(2S,6R)-2-[[[1-[2-(hydroxymethyl)-6-methyl-4-pyridyl]-4-piperidyl]amino]methyl]-6-methyl-morpholin-4-yl]quinoline-8-carbonitrile;

5-[(2S,6R)-2-[[[(3R)-1-[2-(hydroxymethyl)-6-methyl-4-pyridyl]-3-piperidyl]amino]methyl]-6-methyl-morpholin-4-yl]quinoline-8-carbonitrile;

5-[(2S,6R)-2-[[[(3S)-1-[2-(hydroxymethyl)-6-methyl-4-pyridyl]-3-piperidyl]amino]methyl]-6-methyl-morpholin-4-yl]quinoline-8-carbonitrile;

trans-5-[(2S,6R)-2-[[[3-hydroxy-1-[2-(hydroxymethyl)-6-methyl-4-pyridyl]-4-piperidyl]amino]methyl]-6-methyl-morpholin-4-yl]quinoline-8-carbonitrile;

trans-5-[(2S,6R)-2-[[[4-hydroxy-1-[2-(hydroxymethyl)-6-methyl-4-pyridyl]-3-piperidyl]amino]methyl]-6-methyl-morpholin-4-yl]quinoline-8-carbonitrile;

5-[(2S,6R)-2-[[[(3R)-1-[2-(hydroxymethyl)-6-methyl-4-pyridyl]pyrrolidin-3-yl]amino]methyl]-6-methyl-morpholin-4-yl]quinoline-8-carbonitrile;

5-[(2S,6R)-2-[[[(3S)-1-[2-(hydroxymethyl)-6-methyl-4-pyridyl]pyrrolidin-3-yl]amino]methyl]-6-methyl-morpholin-4-yl]quinoline-8-carbonitrile;

5-[(2S,6R)-2-[[[1-[2-(hydroxymethyl)-6-methyl-4-pyridyl]azetidin-3-yl]amino]methyl]-6-methyl-morpholin-4-yl]quinoline-8-carbonitrile;

trans-5-[(2S,6R)-2-[[[4-hydroxy-1-[2-(hydroxymethyl)-6-methyl-4-pyridyl]pyrrolidin-3-yl]amino]methyl]-6-methyl-morpholin-4-yl]quinoline-8-carbonitrile;

trans-5-[(2S,6R)-2-[[[1-(2,6-dimethyl-4-pyridyl)-4-hydroxy-3-piperidyl]amino]methyl]-6-methyl-morpholin-4-yl]quinoline-8-carbonitrile;

cis-5-[(2S,6R)-2-[[[4-fluoro-1-[2-(hydroxymethyl)-6-methyl-4-pyridyl]-3-piperidyl]amino]methyl]-6-methyl-morpholin-4-yl]quinoline-8-carbonitrile;

trans-5-[(2S,6R)-2-[[[4-fluoro-1-[2-(hydroxymethyl)-6-methyl-4-pyridyl]-3-piperidyl]amino]methyl]-6-methyl-morpholin-4-yl]quinoline-8-carbonitrile;

5-[(2S,6R)-2-[[[(3R)-1-[2-(hydroxymethyl)-6-methyl-4-pyridyl]-3-piperidyl]-methyl-amino]methyl]-6-methyl-morpholin-4-yl]quinoline-8-carbonitrile;

5-[(2S,6R)-2-[[[5,5-difluoro-1-[2-(hydroxymethyl)-6-methyl-4-pyridyl]-3-piperidyl]amino]methyl]-6-methyl-morpholin-4-yl]quinoline-8-carbonitrile;

5-[(2S,6R)-2-[[[1-[2-(hydroxymethyl)-6-methyl-4-pyridyl]azetidin-3-yl]-methyl-amino]methyl]-6-methyl-morpholin-4-yl]quinoline-8-carbonitrile;

5-[(2S,6R)-2-[[[(3S)-1-[2-(hydroxymethyl)-6-methyl-4-pyridyl]-3-piperidyl]-methyl-amino]methyl]-6-methyl-morpholin-4-yl]quinoline-8-carbonitrile;

4-[(2S,6R)-2-[[2-benzyl-4-(2,6-dimethyl-4-pyridyl)piperazin-1-yl]methyl]-6-methyl-morpholin-4-yl]pyrazolo[1,5-a]pyridine-7-carbonitrile;

5-[(2S,6R)-2-[[4-(2-amino-6-methyl-4-pyridyl)piperazin-1-yl]methyl]-6-methyl-morpholin-4-yl]quinoline-8-carbonitrile;

5-[(2S,6R)-2-[[(2R)-4-(2-amino-6-methyl-4-pyridyl)-2-(hydroxymethyl)piperazin-1-yl]methyl]-6-methyl-morpholin-4-yl]quinoline-8-carbonitrile;

5-[(2R,6S)-2-methyl-6-[[4-[2-methyl-6-(methylamino)-4-pyridyl]piperazin-1-yl]methyl]morpholin-4-yl]quinoline-8-carbonitrile;

5-[(2S,6R)-2-[[4-(2-amino-6-methyl-4-pyridyl)-1-piperidyl]methyl]-6-methyl-morpholin-4-yl]quinoline-8-carbonitrile; 5-[(2S,6R)-2-[[2-[2-(1-hydroxyethyl)-6-methyl-4-pyridyl]-2,6-diazaspiro[3.3]heptan-6-yl]methyl]-6-methyl-morpholin-4-yl]quinoline-8-carbonitrile;

5-[(2S,6R)-2-[[4-[2-amino-6-(hydroxymethyl)-4-pyridyl]piperazin-1-yl]methyl]-6-methyl-morpholin-4-yl]quinoline-8-carbonitrile;

5-[(2S,6R)-2-[[2-[2-(1-hydroxyethyl)-4-pyridyl]-2,6-diazaspiro[3.3]heptan-6-yl]methyl]-6-methyl-morpholin-4-yl]quinoline-8-carbonitrile;

5-[(2S,6R)-2-[[2-[2-(difluoromethyl)-4-pyridyl]-2,6-diazaspiro[3.3]heptan-6-yl]methyl]-6-methyl-morpholin-4-yl]quinoline-8-carbonitrile;

5-[(2S,6R)-2-[[2-[2-(1-hydroxy-1-methyl-ethyl)-4-pyridyl]-2,6-diazaspiro[3.3]heptan-6-yl]methyl]-6-methyl-morpholin-4-yl]quinoline-8-carbonitrile;

5-[(2S,6R)-2-[[2-(2-cyclopropyl-4-pyridyl)-2,6-diazaspiro[3.3]heptan-6-yl]methyl]-6-methyl-morpholin-4-yl]quinoline-8-carbonitrile;

5-[(2S,6R)-2-[[4-[2-(hydroxymethyl)-6-methyl-4-pyridyl]piperazin-1-yl]methyl]-6-methyl-morpholin-4-yl]quinoline-8-carbonitrile; and

5-[(2S,6R)-2-[[8-[2-(hydroxymethyl)-4-pyridyl]-5-oxa-2,8-diazaspiro[3.5]nonan-2-yl]methyl]-6-methyl-morpholin-4-yl]quinoline-8-carbonitrile;

or a pharmaceutically acceptable salt, enantiomer or diastereomer thereof.

A number of compounds used as reference herein were disclosed in patent US20150105370 showing TLR7 and TLR9 potency data summarized in table 1. Compounds in Table 1 are all characterized with an aromatic ring at the terminal position (phenyl or pyridinyl), however, according to the potency data disclosed, only some of the compounds in Table 1 showed good TLR7 potency, and all of which were lack of TLR9 potency. More examples disclosed in US20150105370 with same structural characteristics confirmed such trend, which suggests the terminal aryl/heteroaryl ring is not favorable for TLR9 activity.

Meanwhile, more analogues of the compounds disclosed in US20150105370, such as compound R1, compound R2 which bear some substituents on the terminal aryl ring, were synthesized to confirm the SAR (structure-activity-relationship). But according to the potency data shown in Table 2, the substituents on the terminal aryl ring may not necessarily improve the potency of TLR9. Therefore, the skill of the art shall not obtain any incitation from the information disclosed in US20150105370 to further optimize such chemical structures.

Surprisingly, the compounds of this invention significantly improved TLR9 potency (>10 folds compared to ER-888286) while keeping excellent TLR7 and TLR8 potency. In another embodiment, the human microsome stability of the compounds of this invention was improved as compared to the reference compounds R1, R2, ER-887258 and ER-888285 (see Table 6). The compounds of formula (I) or (Ia) also showed good hPBMC, cytotoxicity, solubility and SDPK profiles, as well as low CYP inhibition.

TABLE 1
TLR7 and TLR9 potency of compounds disclosed in U.S. Pat. No. 20150105370

| | | HEK/hTLR7 | HEK/hTLR9 |
|---|---|---|---|
| Compound | Structure | IC50 (μM) | IC50 (μM) |
| ER-887258 | 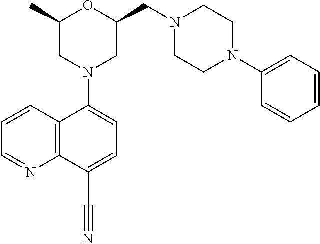 [SMILES: C[C@@H]1CN(c2ccc(C#N)c3ncccc23)C[C@H](CN2CCN(c3ccccc3)CC2)O1] | 0.0852 | >2.0 |
| ER-888285 | 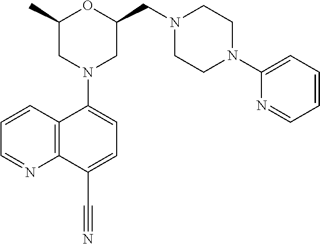 [SMILES: C[C@@H]1CN(c2ccc(C#N)c3ncccc23)C[C@H](CN2CCN(c3ccccn3)CC2)O1] | 0.120 | >2.0 |
| ER-888286 | 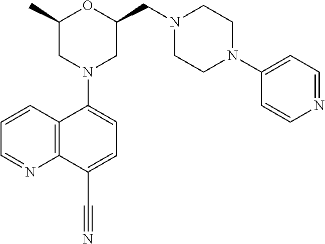 [SMILES: C[C@@H]1CN(c2ccc(C#N)c3ncccc23)C[C@H](CN2CCN(c3ccncc3)CC2)O1] | 1.370 | >2.0 |
| ER-894544 | 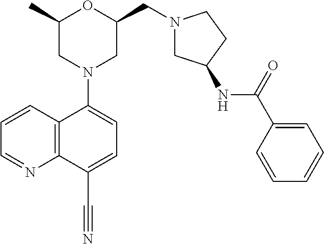 [SMILES: C[C@@H]1CN(c2ccc(C#N)c3ncccc23)C[C@H](CN2CC[C@@H](NC(=O)c3ccccc3)C2)O1] | 0.043 | >6.2 |
| ER-894160 | 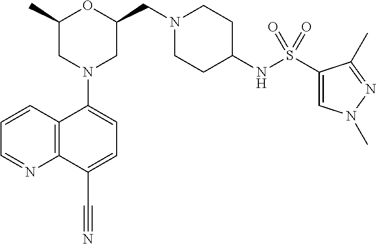 [SMILES: Cc1nn(C)cc1S(=O)(=O)NC1CCN(C[C@H]2CN(c3ccc(C#N)c4ncccc34)C[C@@H](C)O2)CC1] | 0.1990 | >10.0 |
| ER-894155 | 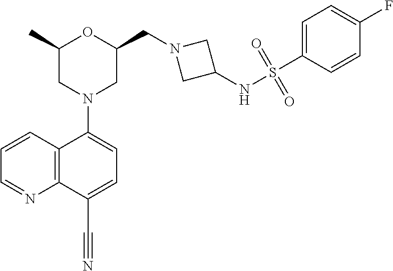 [SMILES: C[C@@H]1CN(c2ccc(C#N)c3ncccc23)C[C@H](CN2CC(NS(=O)(=O)c3ccc(F)cc3)C2)O1] | 0.2820 | >10.0 |

### SYNTHESIS

The compounds of the present invention can be prepared by any conventional means. Suitable processes for synthesizing these compounds as well as their starting materials are provided in the schemes below and in the examples. All substituents, in particular, R¹ to R⁴ are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

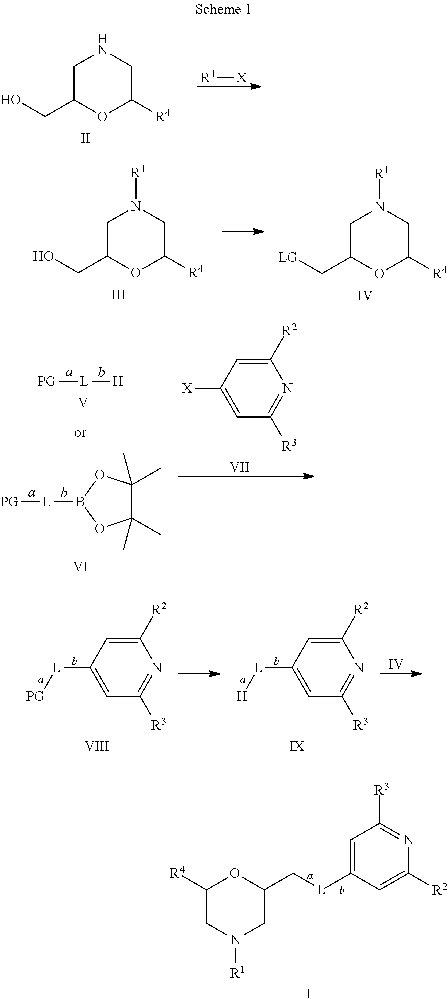

Wherein X is halogen; LG is a leaving group, such as OTf, OTs and OMs; PG is a protecting group, such as Boc and Cbz.

The coupling of compound of formula (II) with R¹-X can be achieved by direct coupling in the presence of a base, such as DIPEA or K₂ CO₃, or under Buchwald-Hartwig amination conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as RuPhos Pd G2, and a base, such as Cs₂CO₃, to provide compound of formula (III). Subsequently the hydroxy group of compound of formula (III) is converted to a leaving group, such as OTf, OTs, or OMs, under basic condition, such as DIPEA, TEA, K₂CO₃ or 2,6-dimethylpyridine, with Tf₂O, TsCl or MsCl. The coupling of compound of formula (V) with compound of formula (VII) can be achieved by direct coupling under high temperature, or under Buchwald-Hartwig amination conditions with a catalyst, such as RuPhos Pd G2, BrettPhos Pd G3, Pd₂(dba)₃/BINAP or Pd₂(dba)₃/XantPhos and a base, such as Cs₂CO₃ or t-BuONa, to provide compound of formula (VIII). The coupling of compound of formula (VI) with compound of formula (VII) can be achieved by Suzuki coupling reaction with a catalyst, such as PdCl₂(dppf) or PdCl₂(dtbpf) and a base, such as K₂CO₃ or Na₂CO₃, followed by hydrogenation reaction with a catalyst, such as Pd-C to provide compound of formula (VIII). The protecting group of compound of formula (VIII) can be removed under high temperature or acidic condition, such as TFA, or hydrogenation condition with a catalyst, such as Pd-C. Compound of formula (IX) was further coupled with compound of formula (IV) in the presence of base, such as K₂ CO₃, DIPEA, or Cs₂CO₃, to afford compound of formula (I). In some embodiment, the coupling of compound of formula (IX) and (IV) may give a product containing a protecting group, e.g. Boc, originated from compound of formula (IX), which will be removed before affording the final compound of formula (I). Compounds of formula (Ia) can be synthesized using the chiral compound of formula (II) correspondingly.

Compounds of this invention can be obtained as mixtures of diastereomers or enantiomers, which can be separated by methods well known in the art, e.g. (chiral) HPLC or SFC.

This invention also relates to a process for the preparation of a compound of formula (I) or (Ia) comprising any of the following step:

a) the coupling of compound of formula (IX),

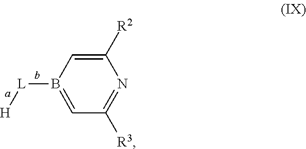

with compound of formula (IV) in the presence of a base;

wherein the base can be for example K₂ CO₃, DIPEA, or Cs₂CO_{3.}

A compound of formula (I) or (Ia) when manufactured according to the above process is also an object of the invention.

INDICATIONS AND METHODS OF TREATMENT

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### EXAMPLES

The invention will be more fully understood by reference to the following examples. They should not, however, be construed as limiting the scope of the invention.

### ABBREVIATIONS

The invention will be more fully understood by reference to the following examples. They should not, however, be construed as limiting the scope of the invention.

Abbreviations used herein are as follows:
ACN: acetonitrile
BINAP: (2,2′-bis(diphenylphosphino)-1,1′-binaphthyl)
Boc₂O: di-tert butyl dicarbonate
BrettPhos Pd G3: [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate
t-Bu XPhosPd G3: [(2-di-tert-butylphosphino-2′,4′,6′-triisopropyl-1,1′-biphenyl)-2-(2′-amino-1,1′-biphenyl)]palladium(II) methanesulfonate
CbzCl: benzylchloroformate
cataCXium A Pd G2: chloro[(di(1-alamantyl)-N-butylphosphine)-2-(2-aminobiphenyl)]palladium(II)
DAST: diethylaminosulfur trifluoride
DCM: dichloromethane
DIAD: diisopropyl azodicarboxylate
DIPEA: N,N-diisopropylethylamine
DMA: dimethylacetamide
DMEDA: 1,2-dimethylethylenediamine
EtOAc or EA: ethyl acetate
FA: formic acid
HLM human liver microsome
IC₅₀: half inhibition concentration
IPA: isopropanol
LCMS liquid chromatography-mass spectrometry
MS: mass spectrometry
NMP: N-methylpyrrolidin-2-one
PdCl₂(dtbpf) 1,1′-bis(di-tert-butylphosphino)ferrocene palladium dichloride
Pd₂(dba)₃: tris(dibenzylideneacetone)dipalladium(0)
PdCl₂(dppf): 1,1′-bis(diphenylphosphino)ferrocene palladium dichloride
PE: petroleum ether
prep-HPLC: preparative high performance liquid chromatography
prep-TLC: preparative thin layer chromatography
PPh₃: triphenylphosphine
Rf: retention factor
rt: room temperature
RuPhos Pd G2: chloro(2-dicyclohexylphosphino-2′,6′-diisopropoxy-1, biphenyl)[2-(2′-amino-1,1′-biphenyl)]palladiurn(II) 2nd generation
SelectFluor: 1-chloromethyl-4-fluoro-1,4-diazoniabicyclo[2.2.2]octane bis(tetrafluoroborate)
SFC: supercritical fluid chromatography
TEA: triethylamine
TFA: trifluoroacetic acid
Tf₂O: trifluoromethanesulfonic anhydride
THF: tetrahydrofuran
v/v: volume ratio
XantPhos: 4,5-Bis(diphenylphosphino)-9,9-dimethylxanthene
XPhos: 2-dicyclohexylphosphino-2′,4′,6′-triisopropylbiphenyl
XPhos Pd G2: chloro(2-dicyclohexylphosphino-2′,4′6′-triisopropyl-1,1-biphenyl) [2-(2′-amino-1,1′-biphenyl)]palladium(II)

### GENERAL EXPERIMENTAL CONDITIONS

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 ∈, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃.H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):

Acidic condition I: A: 0.1% TFA in H₂O; B: 0.1% TFA in acetonitrile;

Acidic condition II: A: 0.0375% TFA in H₂O; B: 0.01875% TFA in acetonitrile;

Basic condition I: A: 0.1% NH₃.H₂O in H₂O; B: acetonitrile;

Basic condition II: A: 0.025% NH₃.H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate A [(2R,6R)-4-(8-cyano-5-quinolyl)-6-methyl-morpholin-2-yl]methyl trifluoromethanesulfonate

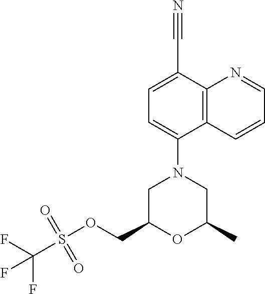 [SMILES: C[C@@H]1CN(c2ccc(C#N)c3ncccc23)C[C@H](COS(=O)(=O)C(F)(F)F)O1]

The title compound was prepared according to the following scheme:

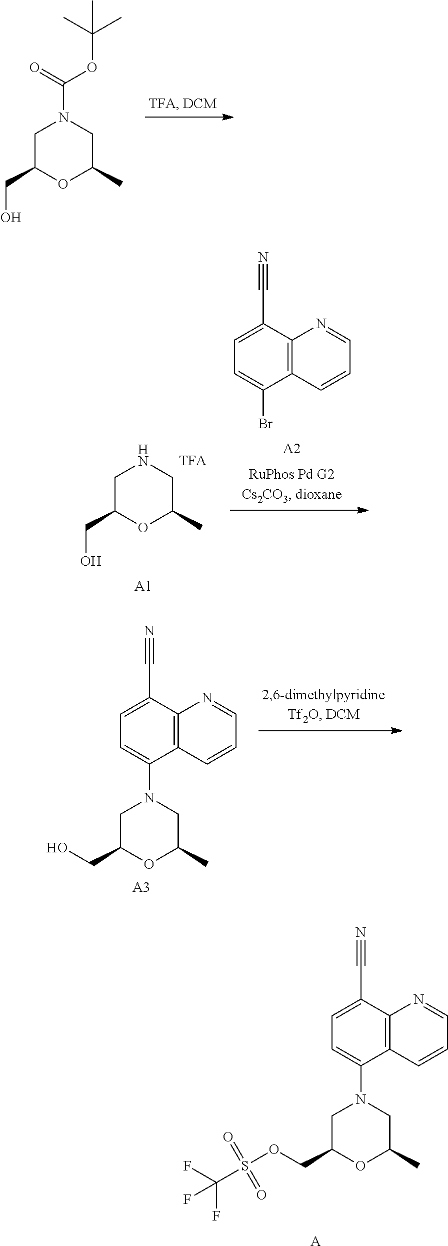

Step 1: preparation of [(2R,6R)-6-methylmorpholin-2-yl]methanol;2,2,2-trifluoroacetic acid (compound A1)

To a solution of tert-butyl(2R,6R)-2-(hydroxymethyl)-6-methylmorpholine-4-carboxylate (CAS: 1700609-48-8, Vendor: WuXi Apptec, 1.35 g, 5.84 mmol) in DCM (10 mL) was added 2,2,2-trifluoroacetic acid (2.66 g, 23.30 mmol). The reaction mixture was stirred at rt for 3 hrs.

Then the reaction mixture was concentrated in vacuo to give the crude product compound A1 (1.43 g) which was used in next step directly. MS: calc'd 132 (MH⁺), measured 132 (MH⁺).

Step 2: preparation of 5-[(2R,6R)-2-(hydroxymethyl)-6-methyl-morpholin-4-yl]quinoline-8-carbonitrile (compound A3)
A mixture of 5-bromoquinoline-8-carbonitrile (compound A2, CAS: 507476-70-2, Vendor: BePharm, 1.50 g, 6.42 mmol), [(2R,6R)-6-methylmorpholin-2-yl]methanol;2,2,2-trifluoroacetic acid (compound A1, 1.43 g, 5.83 mmol), RuPhos Pd G2 (136 mg, 175 μmol) and Cs₂CO₃ (5.70 g, 17.50 mmol) in 1,4-dioxane (10 mL) was heated to 90° C. overnight under N₂. After being cooled down, the solid was filtered off and washed with EA (10 mL) twice. The combined organics was concentrated, the residue was purified by flash column eluting with a gradient of EA/PE (0 to 100%) to afford compound A3 (0.71 g) as a light yellow solid. MS: calc'd 284 (MH⁺), measured 284 (MH⁺).

Step 3: preparation of [(2R,6R)-4-(8-cyano-5-quinolyl)-6-methyl-morpholin-2-yl]methyl trifluoromethanesulfonate (Intermediate A)
To a flask was added 5-[(2R,6R)-2-(hydroxymethyl)-6-methyl-morpholin-4-yl]quinoline-8-carbonitrile (compound A3, 0.71 g, 2.50 mmol), DCM (10 mL) and 2,6-dimethylpyridine (0.54 g, 577 μL, 5.00 mmol). Then the reaction mixture was cooled with ice bath and trifluoromethanesulfonic anhydride (1.06 g, 634 μL, 3.75 mmol) was added dropwise. After being stirred for 2 hrs, the mixture was concentrated and purified by flash column (EA/PE=0 to 40%) to give product Intermediate A (0.72 g) as a yellow solid. MS: calc'd 416 (MH₊), measured 416 (MH₊).

### Intermediate B [(2R,6R)-4-(7-cyanopyrazolo[1,5-a]pyridin-4-yl)-6-methyl-morpholin-2-yl]methyl trifluoromethanesulfonate

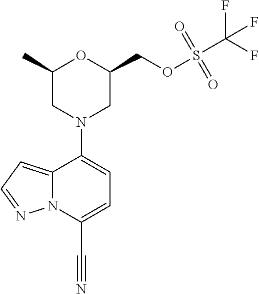 [SMILES: C[C@@H]1CN(c2ccc(C#N)n3nccc23)C[C@H](COS(=O)(=O)C(F)(F)F)O1]

The title compound was prepared in analogy to the preparation of Intermediate A by using 4-chloropyrazolo[1,5-a]pyridine-7-carbonitrile (CAS: 1268520-74-6, Vendor: PharmaBlock) instead of 5-bromoquinoline-8-carbonitrile (compound A2). Intermediate B (116 mg) was obtained as a white solid. MS: calc'd 405 (MH₊), measured 405 (MH₊).

### Reference Compound R1

### 5-[(2S,6R)-2-[[4-(4-methoxyphenyl)piperazin-1-yl]methyl]-6-methyl-morpholin-4-yl]quinoline-8-carbonitrile

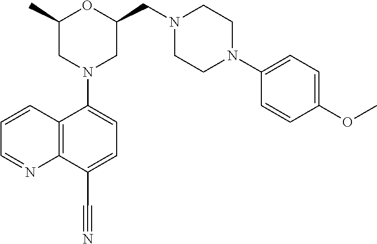 [SMILES: COc1ccc(N2CCN(C[C@H]3CN(c4ccc(C#N)c5ncccc45)C[C@@H](C)O3)CC2)cc1]

The title compound was prepared in analogy to the step 3 in preparation of Example 1 by using 1-(4-cholophenyl)piperazine (CAS: 38212-33-8, Vendor: BePharm) instead of [4-(2,6-diazaspiro[3.3]heptan-2-yl)-6-methyl-2-pyridyl]methanol;2,2,2-trifluoroacetic acid (compound 1d). Compound R1 (22 mg) was obtained as a yellow solid. MS: calc'd 458 (MH₊), measured 458 (MH₊). ¹H NMR (400 MHz, METHANOL-d₄) δ=9.00 (dd, J=1.7, 4.2 Hz, 1H), 8.67 (dd, J=1.7, 8.6 Hz, 1H), 8.18 (d, J=8.1 Hz, 1H), 7.66 (dd, J=4.3, 8.6 Hz, 1H), 7.30 (d, J=7.9 Hz, 1H), 7.05-6.98 (m, 2H), 6.93-6.85 (m, 2H), 4.55-4.46 (m, 1H), 4.26-4.16 (m, 1H), 3.89-3.56 (m, 7H), 3.50-3.37 (m, 6H), 3.25-3.00 (m, 2H), 2.84-2.71 (m, 2H), 1.33 (d, J=6.2 Hz, 3H).

### Reference Compound R2

### 5-[(2S,6R)-2-[[4-(4-chlorophenyl)piperazin-1-yl]methyl]-6-methyl-morpholin-4-yl]quinoline-8-carbonitrile

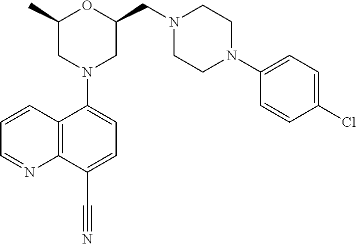 [SMILES: C[C@@H]1CN(c2ccc(C#N)c3ncccc23)C[C@H](CN2CCN(c3ccc(Cl)cc3)CC2)O1]

The title compound was prepared in analogy to the preparation of compound R1 by using 1-(4-cholophenyl)piperazine (CAS: 38212-33-8, Vendor: BePharm) instead of 1-(4-methoxyphenyl)piperazine. Compound R2 (24 mg) was obtained as a yellow solid. MS: calc'd 462 (MH₊), measured 462 (MH₊). ¹H NMR (400 MHz, METHANOL-d₄) δ=9.00 (dd, J=1.6, 4.3 Hz, 1H), 8.67 (dd, J=1.7, 8.6 Hz, 1H), 8.17 (d, J=8.1 Hz, 1H), 7.66 (dd, J=4.3, 8.6 Hz, 1H), 7.34-7.26 (m, 3H), 7.07-6.98 (m, 2H), 4.55-4.47 (m, 1H), 4.25-4.15 (m, 1H), 3.99-3.55 (m, 3H), 3.55-3.32 (m, 7H), 3.28-3.04 (m, 2H), 2.85-2.71 (m, 2H), 1.33 (d, J=6.2 Hz, 3H).

### Example 1

### 5-[(2S,6R)-2-[[2-[2-(hydroxymethyl)-6-methyl-4-pyridyl]-2,6-diazaspiro[3.3]heptan-6-yl]methyl]-6-methyl-morpholin-4-yl]quinoline-8-carbonitrile

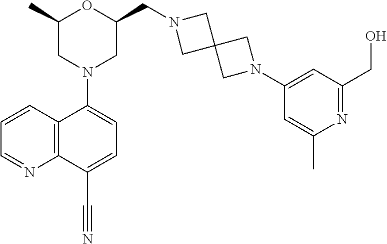 [SMILES: Cc1cc(N2CC3(CN(C[C@H]4CN(c5ccc(C#N)c6ncccc56)C[C@@H](C)O4)C3)C2)cc(CO)n1]

The title compound was prepared according to the following scheme:

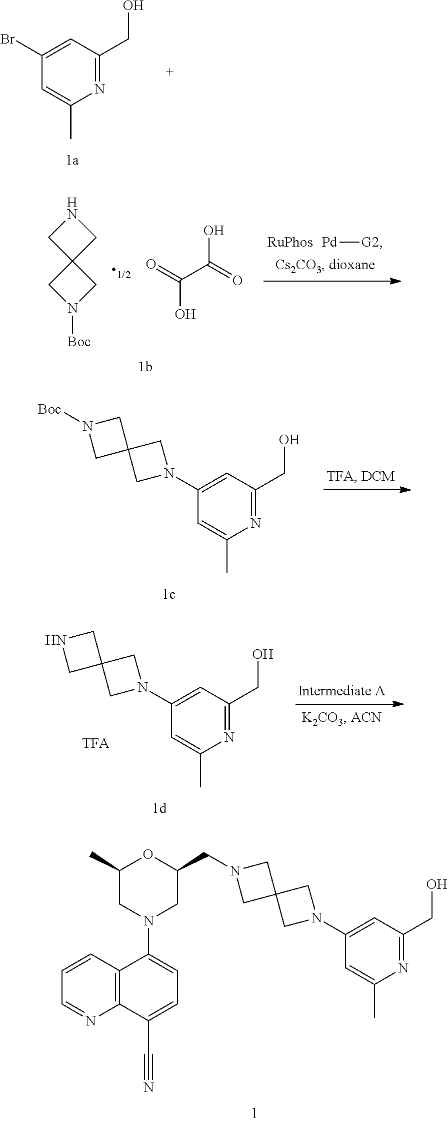

### Step 1: preparation of tert-butyl 6-[2-(hydroxymethyl)-6-methyl-4-pyridyl]-2,6-diazaspiro[3.3]heptane-2-carboxylate (compound 1c)

To a flask was added (4-bromo-6-methyl-2-pyridyl)methanol (compound 1a, CAS: 448906-60-3, Vendor: BePharm, 150 mg, 74 μmol), tert-butyl 2,6-diazaspiro[3.3]heptane-2-carboxylate;oxalic acid (compound 1b, CAS: 1041026-70-3, Vendor: BePharm, 235 mg, 968 μmol), Cs₂CO₃ (726 mg, 2.23 mmol) and 1,4-dioxane (5 mL), the suspension was bubbled with N₂ for 5 mins and Ruphos Pd G2 (29 mg, 37 μmol) was added. The mixture was heated to 120° C. under microwave for 3 hrs. After being cooled down, the mixture was diluted with 10 mL EA and filtered through celite, the filtrate was concentrated and purified by flash column (MeOH/DCM=0 to 10%) to give compound 1c (72 mg) as a yellow oil. MS: calc'd 320 (MH₊), measured 320 (MH₊).

Step 2: preparation of [4-(2,6-diazaspiro[3.3]heptan-2-yl)-6-methyl-2-pyridyl]methanol;2,2,2-trifluoroacetic acid (compound 1d)

To a solution of tert-butyl 6-[2-(hydroxymethyl)-6-methyl-4-pyridyl]-2,6-diazaspiro[3.3]heptane-2-carboxylate (compound alc, 72 mg, 225 μmol) in DCM (5 mL) was added TFA (1 mL). After being stirred at rt for 3 hrs, the reaction mixture was concentrated in vacuo to give the crude product compound 1d (75 mg) as a yellow oil which was used in next step directly. MS: calc'd 220 (MH₊), measured 220 (MH₊).

Step 3: preparation of 5-[(2S,6R)-2-[[2-[2-(hydroxymethyl)-6-methyl-4-pyridyl]-2,6-diazaspiro[3.3]heptan-6-yl]methyl]-6-methyl-morpholin-4-yl]quinoline-8-carbonitrile (Equation 1)

To a tube was added [(2R,6R)-4-(8-cyano-5-quinolyl)-6-methyl-morpholin-2-yl]methyl trifluoromethanesulfonate (Intermediate A, 50 mg, 120 μmol), [4-(2,6-diazaspiro[3.3]heptan-2-yl)-6-methyl-2-pyridyl]methanol; 2,2,2-trifluoroacetic acid (compound 1d, 75 mg, 225 μmol), potassium carbonate (83 mg, 602 μmol) and ACN (6 mL). The reaction mixture was heated to 55° C. for 2 hrs. After being cooled down, the mixture was diluted with some ACN and filtered through celite, the filtrate was concentrated to give a yellow oil which was purified by prep-HPLC to give Example 1 (40 mg) as a light yellow solid. MS: calc'd 485 (MH₊), measured 485 (MH₊). ¹H NMR (400 MHz, METHANOL-d₄) δ=8.88 (dd, J=1.6, 4.3 Hz, 1H), 8.54 (dd, J=1.6, 8.6 Hz, 1H), 8.05 (d, J=7.9 Hz, 1H), 7.54 (dd, J=4.2, 8.6 Hz, 1H), 7.16 (d, J=8.1 Hz, 1H), 6.40 (s, 1H), 6.31 (br s, 1H), 4.54 (s, 2H), 4.52-4.23 (m, 8H), 4.17 (br t, J=9.8 Hz, 1H), 4.08-3.96 (m, 1H), 3.43-3.35 (m, 1H), 3.35-3.24 (m, 3H), 2.72-2.54 (m, 2H), 2.40 (s, 3H), 1.19 (d, J=6.2 Hz, 3H).

### Example 3

### 5-[(2S,6R)-2-[[8-(2-cyclopropyl-4-pyridyl)-5-oxa-2,8-diazaspiro[3.5]nonan-2-yl]methyl]-6-methyl-morpholin-4-yl]quinoline-8-carbonitrile

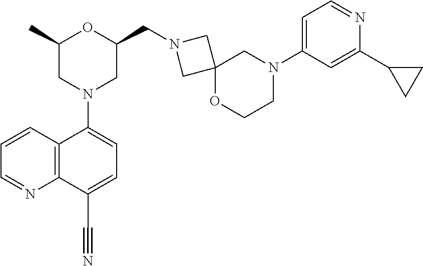 [SMILES: C[C@@H]1CN(c2ccc(C#N)c3ncccc23)C[C@H](CN2CC3(C2)CN(c2ccnc(C4CC4)c2)CCO3)O1]

The title compound was prepared in analogy to the preparation of Example 1 by using 4-bromo-2-cyclopropyl-pyridine (CAS: 1086381-28-3, Vendor: BePharm) and tert-butyl 5-oxa-2,8-diazaspiro[3.5]nonane-2-carboxylate (CAS: 1251011-05-8, Vendor: PharmaBlock) instead of (4-bromo-6-methyl-2-pyridyl)methanol (compound 1a) and tert-butyl 2,6-diazaspiro[3.3]heptane-2-carboxylate;oxalic acid (compound 1b). Example 3 (23 mg) was obtained as a light yellow solid. MS: calc'd 511 (MH₊), measured 511 (MH₊). ¹H NMR (400 MHz, METHANOL-d₄) δ=8.86 (dd, J=1.6, 4.3 Hz, 1H), 8.53 (dd, J=1.7, 8.6 Hz, 1H), 8.04 (d, J=8.1 Hz, 1H), 7.94-7.82 (m, 1H), 7.52 (dd, J=4.2, 8.6 Hz, 1H), 7.14 (d, J=8.1 Hz, 1H), 6.65-6.56 (m, 2H), 4.01-3.85 (m, 2H), 3.68-3.59 (m, 2H), 3.50-3.37 (m, 4H), 3.27 (br d, J=12.3 Hz, 2H), 3.19 (br s, 2H), 3.04 (dd, J=6.4, 8.1 Hz, 2H), 2.69-2.48 (m, 4H), 1.92-1.83 (m, 1H), 1.14 (d, J=6.2 Hz, 3H), 0.91-0.76 (m, 4H).

### Example 4

### 5-[2S,6R)-2-[[4-[2-(hydroxymethyl)-6-methyl-4-pyridyl]-2-methyl-piperazin-1-yl]methyl]-6-methyl-morpholin-4-yl]quinoline-8-carbonitrile

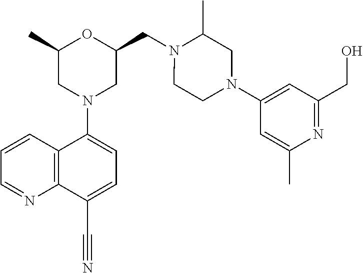 [SMILES: Cc1cc(N2CCN(C[C@H]3CN(c4ccc(C#N)c5ncccc45)C[C@@H](C)O3)C(C)C2)cc(CO)n1]

The title compound was prepared in analogy to the preparation of Example 1 by using tert-butyl 2-methylpiperazine-1-carboxylate (CAS: 120737-78-2, Vendor: Accela ChemBio Inc) instead of tert-butyl 2,6-diazaspiro[3.3]heptane-2-carboxylate;oxalic acid (compound 1b).

### Example 4A and 4B

### 5-[2S,6R)-2-[[(2S)-4-[2-(hydroxymethyl)-6-methyl-4-pyridyl]-2-methyl-piperazin-1-yl]methyl]-6-methyl-morpholin-4-yl]quinoline-8-carbonitrile and 5-[2S,6R)-2-[[(2R)-4-[2-(hydroxymethyl)-6-methyl-4-pyridyl]-2-methyl-piperazin-1-yl]methyl]-6-methyl-morpholin-4-yl]quinoline-8-carbonitrile

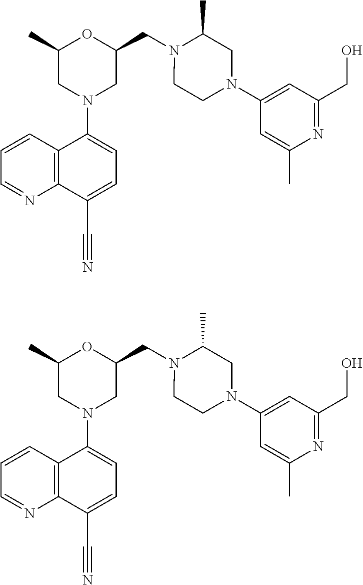 [SMILES: Cc1cc(N2CCN(C[C@H]3CN(c4ccc(C#N)c5ncccc45)C[C@@H](C)O3)[C@@H](C)C2)cc(CO)n1 ; Cc1cc(N2CCN(C[C@H]3CN(c4ccc(C#N)c5ncccc45)C[C@@H](C)O3)[C@H](C)C2)cc(CO)n1]

Example 4A (2 mg) and Example 4B (4 mg) were obtained through prep-HPLC separation of Example 4.

Example 4A: MS: calc'd 487 (MH₊), measured 487 (MH₊). ¹H NMR (400 MHz, METHANOL-d₄) δ=9.02 (dd, J=1.6, 4.2 Hz, 1H), 8.69 (dd, J=1.6, 8.6 Hz, 1H), 8.20 (d, J=8.1 Hz, 1H), 7.68 (dd, J=4.3, 8.6 Hz, 1H), 7.31 (d, J=8.1 Hz, 1H), 7.16 (s, 1H), 7.11 (s, 1H), 4.74 (s, 2H), 4.49 (br t, J=9.8 Hz, 1H), 4.37-4.16 (m, 3H), 3.93-3.75 (m, 2H), 3.75-3.64 (m, 2H), 3.63-3.52 (m, 2H), 3.46 (br d, J=11.9 Hz, 2H), 3.41-3.37 (m, 1H), 2.89-2.71 (m, 2H), 2.60 (s, 3H), 1.52 (br d, J=5.7 Hz, 3H), 1.34 (d, J=6.4 Hz, 3H).

Example 4B: MS: calc'd 487 (MH₊), measured 487 (MH₊). ¹H NMR (400 MHz, METHANOL-d₄) δ=9.02 (dd, J=1.7, 4.2 Hz, 1H), 8.69 (dd, J=1.6, 8.6 Hz, 1H), 8.19 (d, J=7.9 Hz, 1H), 7.68 (dd, J=4.3, 8.6 Hz, 1H), 7.31 (d, J=8.1 Hz, 1H), 7.13 (s, 1H), 7.09 (s, 1H), 4.73 (s, 2H), 4.47 (br t, J=9.9 Hz, 1H), 4.30-4.14 (m, 3H), 3.93-3.76 (m, 2H), 3.75-3.58 (m, 2H), 3.57-3.41 (m, 5H), 2.86-2.71 (m, 2H), 2.59 (s, 3H), 1.49 (br d, J=5.7 Hz, 3H), 1.32 (d, J=6.2 Hz, 3H).

### Example 5

### 5-[2S,6R)-2-[[4-[[2-(hydroxymethyl)-6-methyl-4-pyridyl]amino]-1-piperidyl]methyl]-6-methyl-morpholin-4-yl]quinoline-8-carbonitrile

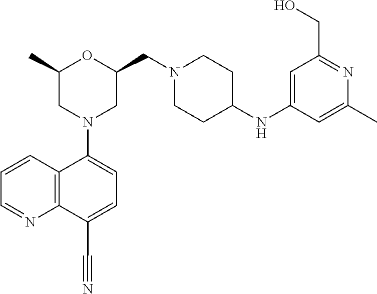 [SMILES: Cc1cc(NC2CCN(C[C@H]3CN(c4ccc(C#N)c5ncccc45)C[C@@H](C)O3)CC2)cc(CO)n1]

The title compound was prepared according to the following scheme:

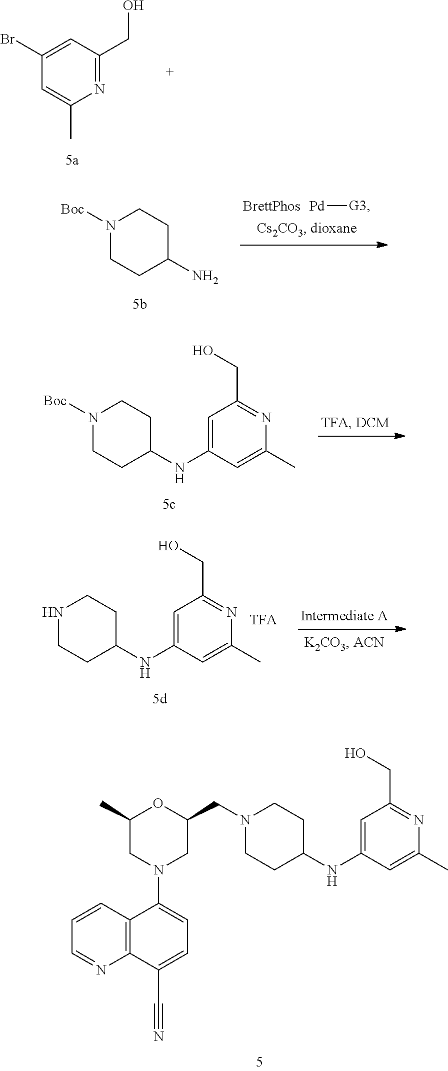

Step 1: preparation of tert-butyl 4-[[2-(hydroxymethyl)-6-methyl-4-pyridyl]amino]piperidine-1-carboxylate (compound 5c)

To a flask was added (4-bromo-6-methyl-2-pyridyl)methanol (compound 5a, CAS: 448906-60-3, Vendor: BePharm, 150 mg, 742 μmol), tert-butyl 4-aminopiperidine-1-carboxylate (compound 5b, CAS: 87120-72-7, Vendor: BePharm, 223 mg, 1.11 mmol), Cs₂CO₃ (726 mg, 2.23 mmol) and 1,4-dioxane (5 mL), the suspension was bubbled with N₂ for 5 mins and BrettPhos Pd-G3 (20 mg, 22 μmol) was added. The mixture was heated to 100° C. under microwave for 2 hrs. After being cooled down, the mixture was diluted with 10 mL EA and filtered through celite, the filtrate was concentrated to give a brown oil, which was purified by flash column (MeOH/DCM=0 to 15%) to give compound 5c (72 mg) as a yellow oil. MS: calc'd 322 (MH₊), measured 322 (MH₊).

Step 2: preparation of [6-methyl-4-(4-piperidylamino)-2-pyridyl]methanol;2,2,2-trifluoroacetic acid (compound 5d)

To a solution of tert-butyl 4-[[2-(hydroxymethyl)-6-methyl-4-pyridyl]amino]piperidine-1-carboxylate (compound 5c, 72 mg, 224 μmol) in DCM (5 mL) was added TFA (1 mL). The reaction mixture was stirred at rt for 3 hrs. Then it was concentrated in vacuo to give the crude product compound 5d (75 mg) which was used in next step directly. MS: calc'd 222 (MH₊), measured 222 (MH₊).

Step 3: preparation of 5-[(2S,6R)-2-[[4-[[2-(hydroxymethyl)-6-methyl-4-pyridyl]amino]-1-piperidyl]methyl]-6-methyl-morpholin-4-yl]quinoline-8-carbonitrile

### Example 5

To a tube was added [(2R,6R)-4-(8-cyano-5-quinolyl)-6-methyl-morpholin-2-yl]methyl trifluoromethanesulfonate (Intermediate A, 40 mg, 96 μmol), [6-methyl-4-(4-piperidylamino)-2-pyridyl]methanol; 2,2,2-trifluoroacetic acid (compound 5d, 75 mg, 224 μmol), potassium carbonate (67 mg, 481 μmol) and ACN (5 mL). The mixture was heated to 50° C. for 2 hrs. After being cooled down, the mixture was diluted with some ACN and filtered through celite, the filtrate was concentrated and purified by prep-HPLC to give the desired product Example 5 (19 mg) as a light yellow solid. MS: calc'd 487 (MH₊), measured 487 (MH₊). ¹H NMR (400 MHz, METHANOL-d₄) δ=9.00 (dd, J=1.6, 4.3 Hz, 1H), 8.67 (dd, J=1.6, 8.6 Hz, 1H), 8.17 (d, J=7.9 Hz, 1H), 7.65 (dd, J=4.3, 8.6 Hz, 1H), 7.27 (d, J=8.1 Hz, 1H), 6.58 (d, J=1.7 Hz, 1H), 6.33 (d, J=1.8 Hz, 1H), 4.52 (s, 2H), 4.26-4.14 (m, 1H), 4.09 (ddd, J=2.3, 6.3, 10.1 Hz, 1H), 3.49-3.37 (m, 3H), 3.13 (br d, J=12.2 Hz, 1H), 2.99 (br d, J=11.4 Hz, 1H), 2.76-2.63 (m, 2H), 2.63-2.45 (m, 2H), 2.41-2.24 (m, 5H), 2.10-1.95 (m, 2H), 1.68-1.51 (m, 2H), 1.27 (d, J=6.2 Hz, 3H).

### Example 6

### 5-[2S,6R)-2-[[3-[[2-(hydroxymethyl)-6-methyl-4-pyridyl]amino]pyrrolidin-1-yl]methyl]-6-methyl-morpholin-4-yl]quinoline-8-carbonitrile

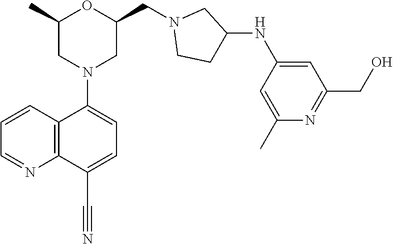 [SMILES: Cc1cc(NC2CCN(C[C@H]3CN(c4ccc(C#N)c5ncccc45)C[C@@H](C)O3)C2)cc(CO)n1]

The title compound was prepared in analogy to the preparation of Example 5 by using tert-butyl 3-aminopyrrolidine-1-carboxylate (CAS: 186550-13-0, Vendor: Fudechem) instead of tert-butyl 4-aminopiperidine-1-carboxylate (compound 5b). Example 6 (4 mg) was obtained as a light yellow solid. MS: calc'd 473 (MH₊), measured 473 (MH₊). ¹H NMR (400 MHz, METHANOL-d₄) δ=9.04-8.96 (m, 1H), 8.67 (d, J=8.6 Hz, 1H), 8.16 (d, J=7.9 Hz, 1H), 7.65 (dd, J=4.2, 8.5 Hz, 1H), 7.26 (d, J=8.1 Hz, 1H), 6.58 (s, 1H), 6.34 (s, 1H), 4.53 (s, 2H), 4.17-4.08 (m, 2H), 3.48-3.38 (m, 3H), 3.07 (dd, J=7.0, 9.8 Hz, 1H), 2.95-2.85 (m, 1H), 2.78-2.58 (m, 6H), 2.45-2.25 (m, 4H), 1.80-1.69 (m, 1H), 1.27 (d, J=6.2 Hz, 3H).

### Example 7

### 5-[2S,6R)-2-[[3-[(2,6-dimethyl-4-pyridyl)amino]azetidin-1-yl]methyl]-6-methyl-morpholin-4-yl]quinoline-8-carbonitrile

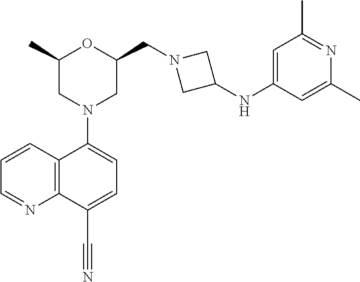 [SMILES: Cc1cc(NC2CN(C[C@H]3CN(c4ccc(C#N)c5ncccc45)C[C@@H](C)O3)C2)cc(C)n1]

The title compound was prepared in analogy to the preparation of Example 5 by using 4-bromo-2,6-dimethyl-pyridine (CAS: 5093-70-9, Vendor: Accela ChemBio Inc) and tert-butyl 3-aminoazetidine-1-carboxylate (CAS: 193269-78-2, Vendor: PharmaBlock) instead of (4-bromo-6-methyl-2-pyridyl)methanol (compound 5a) and tert-butyl 4-aminopiperidine-1-carboxylate (compound 5b). Example 7 (27 mg) was obtained as a yellow solid. MS: calc'd 443 (MH⁺), measured 443 (MH₊). ¹H NMR (400 MHz, METHANOL-d₄) δ=8.99 (dd, J=1.3, 4.2 Hz, 1H), 8.65 (dd, J=1.3, 8.6 Hz, 1H), 8.16 (d, J=7.9 Hz, 1H), 7.65 (dd, J=4.2, 8.6 Hz, 1H), 7.28 (d, J=8.1 Hz, 1H), 6.67 (br s, 2H), 4.85-4.68 (m, 3H), 4.43-4.23 (m, 3H), 4.20-4.08 (m, 1H), 3.64-3.46 (m, 2H), 3.42 (br d, J=11.7 Hz, 2H), 2.84-2.66 (m, 2H), 2.52 (br s, 6H), 1.30 (d, J=6.2 Hz, 3H).

### Example 8

### 5-[2S,6R)-2-[[1-(2,6-dimethyl-4-pyridyl)-1,6-diazaspiro[3.3]heptan-6-yl]methyl]-6-methyl-morpholin-4-yl]quinoline-8-carbonitrile

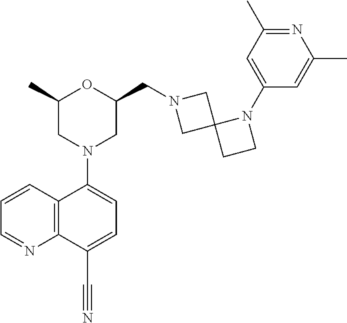 [SMILES: Cc1cc(N2CCC23CN(C[C@H]2CN(c4ccc(C#N)c5ncccc45)C[C@@H](C)O2)C3)cc(C)n1]

The title compound was prepared in analogy to the preparation of Example 1 by using 4-bromo-2,6-dimethyl-pyridine and tert-butyl 1,6-diazaspiro[3.3]heptane-6-carboxylate;oxalic acid (CAS: 1272412-72-2, Vendor: PharmaBlock) instead of (4-bromo-6-methyl-2-pyridyl)methanol (compound 1a) and tert-butyl 2,6-diazaspiro[3.3]heptane-2-carboxylate;oxalic acid (compound 1b). Example 8 (23 mg) was obtained as a light yellow solid. MS: calc'd 469 (MH₊), measured 469 (MH₊). ¹H NMR (400 MHz, METHANOL-d₄) δ=9.02 (dd, J=1.6, 4.3 Hz, 1H), 8.68 (dd, J=1.7, 8.6 Hz, 1H), 8.19 (d, J=7.9 Hz, 1H), 7.67 (dd, J=4.3, 8.6 Hz, 1H), 7.31 (d, J=8.1 Hz, 1H), 7.06 (br s, 1H), 6.47 (br s, 1H), 5.15 (br s, 2H), 4.48 (br d, J=11.6 Hz, 2H), 4.38-4.28 (m, 1H), 4.21-4.03 (m, 3H), 3.65-3.51 (m, 2H), 3.45 (br t, J=10.0 Hz, 2H), 2.94 (t, J=7.4 Hz, 2H), 2.87-2.70 (m, 2H), 2.55 (br s, 6H), 1.32 (d, J=6.2 Hz, 3H).

### Example 9

### 5-[2S,6R)-2[[7-(2,6-dimethyl-4-pyridyl)-2,7-diazaspiro[3.4]octan-2-yl]methyl]-6-methyl-morpholin-4-yl]quinoline-8-carbonitrile

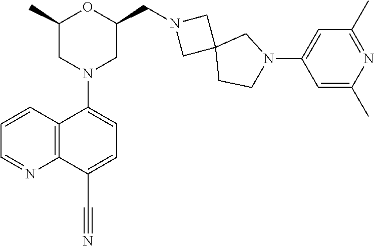 [SMILES: Cc1cc(N2CCC3(CN(C[C@H]4CN(c5ccc(C#N)c6ncccc56)C[C@@H](C)O4)C3)C2)cc(C)n1]

The title compound was prepared in analogy to the preparation of Example 1 by using 4-bromo-2,6-dimethyl-pyridine and tert-butyl 2,7-diazaspiro[3.4]octane-2-carboxylate (CAS: 885270-84-8, Vendor: PharmaBlock) instead of (4-bromo-6-methyl-2-pyridyl)methanol (compound 1a) and tert-butyl 2,6-diazaspiro[3.3]heptane-2-carboxylate;oxalic acid (compound 1b). Example 9 (32 mg) was obtained as a light yellow solid. MS: calc'd 483 (MH₊), measured 483 (MH₊). ¹H NMR (400 MHz, METHANOL-d₄) δ=9.01 (dd, J=1.6, 4.2 Hz, 1H), 8.66 (dd, J=1.6, 8.6 Hz, 1H), 8.18 (d, J=8.1 Hz, 1H), 7.66 (dd, J=4.2, 8.6 Hz, 1H), 7.29 (d, J=8.1 Hz, 1H), 6.65-6.52 (m, 2H), 4.52-4.28 (m, 5H), 4.20-4.09 (m, 1H), 3.98-3.74 (m, 2H), 3.69-3.60 (m, 2H), 3.60-3.47 (m, 2H), 3.42 (br d, J=12.5 Hz, 2H), 2.85-2.64 (m, 2H), 2.52 (br d, J=4.8 Hz, 8H), 1.31 (d, J=6.2 Hz, 3H).

### Example 10

### cis-5-[2S,6R)-2-[[5-[2-(hydroxymethyl)-6-methyl-4-pyridyl]-1,3,3a,4,6,6a-hexahydropyrrolo[3,4-c]pyrrol-2-yl]methyl]-6-methyl-morpholin-4-yl]quinoline-8-carbonitrile

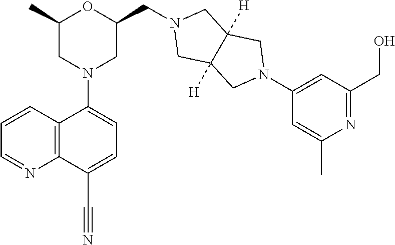 [SMILES: Cc1cc(N2C[C@H]3CN(C[C@H]4CN(c5ccc(C#N)c6ncccc56)C[C@@H](C)O4)C[C@H]3C2)cc(CO)n1]

The title compound was prepared in analogy to the preparation of Example 1 by using cis-tert-butyl 2,3,3a,4,6,6a-hexahydro-1H-pyrrolo[3,4-c]pyrrole-5-carboxylate (compound 10b, CAS: 141449-85-6, Vendor: PharmaBlock) instead of tert-butyl 2,6-diazaspiro[3.3]heptane-2-carboxylate;oxalic acid (compound 1b). Example 10 (42 mg) was obtained as a light yellow solid. MS: calc'd 499 (MH₊), measured 499 (MH₊). ¹H NMR (400 MHz, METHANOL-d₄) δ=8.98 (dd, J=1.6, 4.3 Hz, 1H), 8.63 (dd, J=1.3, 8.5 Hz, 1H), 8.14 (d, J=8.1 Hz, 1H), 7.63 (dd, J=4.3, 8.6 Hz, 1H), 7.26 (d, J=8.1 Hz, 1H), 6.78 (br s, 1H), 6.68 (br s, 1H), 4.70 (s, 2H), 4.42 (br t, J=9.9 Hz, 1H), 4.20-4.09 (m, 2H), 3.98-3.62 (m, 6H), 3.59-3.36 (m, 7H), 2.74 (dt, J=11.1, 12.7 Hz, 2H), 2.55 (s, 3H), 1.32 (d, J=6.2 Hz, 3H).

### Example 11

### 5-[2S,6R)-2-[[3-[(2,6-dimethyl-4-pyridyl)-methyl-amino]azetidin-1-yl]methyl]-6-methyl-morpholin-4-yl]quinoline-8-carbonitrile

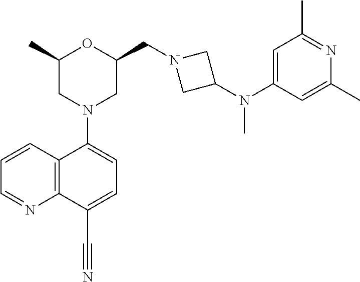 [SMILES: Cc1cc(N(C)C2CN(C[C@H]3CN(c4ccc(C#N)c5ncccc45)C[C@@H](C)O3)C2)cc(C)n1]

The title compound was prepared in analogy to the preparation of Example 1 by using 4-bromo-2,6-dimethyl-pyridine and tert-butyl 3-(methylamino)azetidine-1-carboxylate (CAS: 454703-20-9, Vendor: PharmaBlock) instead of (4-bromo-6-methyl-2-pyridyl)methanol (compound 1a) and tert-butyl 2,6-diazaspiro[3.3]heptane-2-carboxylate;oxalic acid (compound 1b). Example 11 (34 mg) was obtained as a light yellow solid. MS: calc'd 457 (MH₊), measured 457 (MH₊). ¹H NMR (400 MHz, METHANOL-d₄) δ=9.00 (dd, J=1.5, 4.2 Hz, 1H), 8.66 (dd, J=1.6, 8.6 Hz, 1H), 8.16 (d, J=8.1 Hz, 1H), 7.66 (dd, J=4.3, 8.6 Hz, 1H), 7.28 (d, J=8.1 Hz, 1H), 6.87 (s, 2H), 5.30 (br s, 1H), 4.79-4.48 (m, 4H), 4.36 (br t, J=9.9 Hz, 1H), 4.21-4.10 (m, 1H), 3.68-3.49 (m, 2H), 3.43 (br d, J=12.5 Hz, 2H), 3.30 (s, 3H), 2.86-2.68 (m, 2H), 2.57 (s, 6H), 1.31 (d, J=6.2 Hz, 3H).

### Example 12

### 5-[2S,6R)-2-[[1-[2-(hydroxymethyl)-6-methyl-4-pyridyl]-1,6-diazaspiro[3.3]heptan-6-yl]methyl]-6-methyl-morpholin-4-yl]quinoline-8-carbonitrile

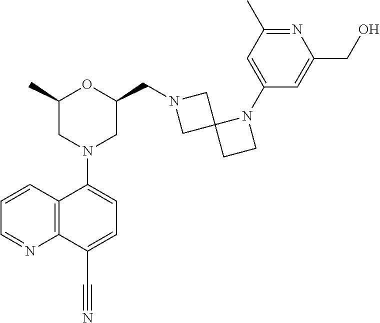 [SMILES: Cc1cc(N2CCC23CN(C[C@H]2CN(c4ccc(C#N)c5ncccc45)C[C@@H](C)O2)C3)cc(CO)n1]

The title compound was prepared in analogy to the preparation of Example 1 by using tert-butyl 1,6-diazaspiro[3.3]heptane-6-carboxylate;oxalic acid instead of tert-butyl 2,6-diazaspiro[3.3]heptane-2-carboxylate;oxalic acid (compound 1b). Example 12 (22 mg) was obtained as a light yellow solid. MS: calc'd 485 (MH₊), measured 485 (MH₊). ¹H NMR (400 MHz, METHANOL-d₄) δ=9.01 (dd, J=1.6, 4.3 Hz, 1H), 8.67 (dd, J=1.6, 8.7 Hz, 1H), 8.19 (d, J=8.1 Hz, 1H), 7.67 (dd, J=4.3, 8.6 Hz, 1H), 7.30 (d, J=8.1 Hz, 1H), 7.09 (br s, 1H), 6.59 (br s, 1H), 5.20 (br s, 2H), 4.75 (br d, J=4.5 Hz, 2H), 4.53 (br d, J=13.3 Hz, 2H), 4.36 (br t, J=9.8 Hz, 1H), 4.22-4.05 (m, 3H), 3.69-3.53 (m, 2H), 3.49-3.39 (m, 2H), 2.96 (t, J=7.5 Hz, 2H), 2.88-2.68 (m, 2H), 2.60 (br s, 3H), 1.32 (d, J=6.2 Hz, 3H).

### Example 13

### 5-[2S,6R)-2-[[2-(2,6-dimethyl-4-pyridyl)-5-oxa-2,8-diazaspiro[3.5]nonan-8-yl]methyl]-6-methyl-morpholin-4-yl]quinoline-8-carbonitrile

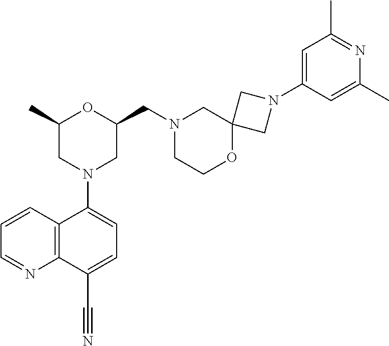 [SMILES: Cc1cc(N2CC3(CN(C[C@H]4CN(c5ccc(C#N)c6ncccc56)C[C@@H](C)O4)CCO3)C2)cc(C)n1]

The title compound was prepared in analogy to the preparation of Example 1 by using 4-bromo-2,6-dimethyl-pyridine and tert-butyl 5-oxa-2,8-diazaspiro[3.5]nonane-8-carboxylate (CAS: 1251005-61-4, Vendor: PharmaBlock) instead of (4-bromo-6-methyl-2-pyridyl)methanol (compound 1a) and tert-butyl 2,6-diazaspiro[3.3]heptane-2-carboxylate;oxalic acid (compound 1b). Example 13 (23 mg) was obtained as a light yellow solid. MS: calc'd 499 (MH₊), measured 499 (MH₊). ¹H NMR (400 MHz, METHANOL-d₄) δ=8.98 (br d, J=3.1 Hz, 1H), 8.64 (br d, J=8.6 Hz, 1H), 8.15 (br d, J=7.9 Hz, 1H), 7.63 (dd, J=4.3, 8.4 Hz, 1H), 7.27 (br d, J=7.9 Hz, 1H), 6.42 (s, 2H), 4.53-4.39 (m, 1H), 4.31 (br d, J=10.3 Hz, 2H), 4.25-4.09 (m, 3H), 4.02 (br s, 2H), 3.74-3.37 (m, 5H), 3.26-3.11 (m, 3H), 2.75 (td, J=11.1, 17.1 Hz, 2H), 2.49 (s, 6H), 1.30 (br d, J=6.1 Hz, 3H).

### Example 14

### 5-[2S,6R)-2-[[8-(2,6-dimethyl-4-pyridyl)-5-oxa-2,8-diazaspiro[3.5]nonan-2-yl]methyl]-6-methyl-morpholin-4-yl]quinoline-8-carbonitrile

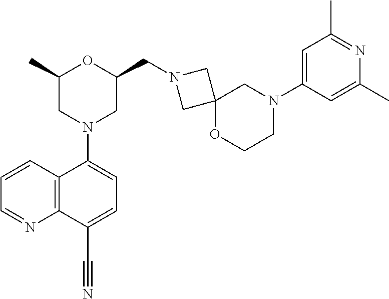 [SMILES: Cc1cc(N2CCOC3(CN(C[C@H]4CN(c5ccc(C#N)c6ncccc56)C[C@@H](C)O4)C3)C2)cc(C)n1]

The title compound was prepared in analogy to the preparation of Example 1 by using 4-bromo-2,6-dimethyl-pyridine and tert-butyl 5-oxa-2,8-diazaspiro[3.5]nonane-2-carboxylate (CAS: 1251011-05-8, Vendor: PharmaBlock) instead of (4-bromo-6-methyl-2-pyridyl)methanol (compound 1a) and tert-butyl 2,6-diazaspiro[3.3]heptane-2-carboxylate;oxalic acid (compound 1b). Example 14 (28 mg) was obtained as a white solid. MS: calc'd 499 (MH₊), measured 499 (MH₊). ¹H NMR (400 MHz, METHANOL-d₄) δ=8.98 (br d, J=3.1 Hz, 1H), 8.65 (br d, J=8.4 Hz, 1H), 8.16 (d, J=8.1 Hz, 1H), 7.63 (dd, J=4.3, 8.6 Hz, 1H), 7.26 (d, J=8.1 Hz, 1H), 6.65 (s, 2H), 4.12-3.99 (m, 2H), 3.75 (br t, J=4.7 Hz, 2H), 3.60-3.48 (m, 4H), 3.39 (br d, J=12.0 Hz, 2H), 3.31-3.25 (m, 2H), 3.22-3.10 (m, 2H), 2.82-2.60 (m, 4H), 2.40 (s, 6H), 1.26 (d, J=6.1 Hz, 3H).

### Example 15

### 5-[2R,6S)-2-methyl-6-[[1-(2-methyl-4-pyridyl)-1,6-diazaspiro[3.3]heptan-6-yl]methyl]morpholin-4-yl]quinoline-8-carbonitrile

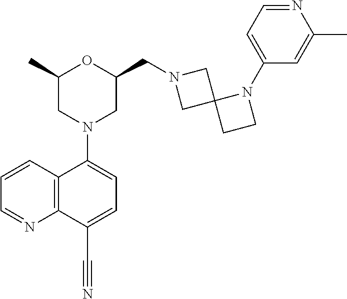 [SMILES: Cc1cc(N2CCC23CN(C[C@H]2CN(c4ccc(C#N)c5ncccc45)C[C@@H](C)O2)C3)ccn1]

The title compound was prepared in analogy to the preparation of Example 1 by using 4-bromo-2-methyl-pyridine (CAS: 22282-99-1, Vendor: TCI) and tert-butyl 1,6-diazaspiro[3.3]heptane-6-carboxylate;oxalic acid instead of (4-bromo-6-methyl-2-pyridyl)methanol (compound 1a) and tert-butyl 2,6-diazaspiro[3.3]heptane-2-carboxylate;oxalic acid (compound 1b). Example 15 (34 mg) was obtained as a yellow solid. MS: calc'd 455 (MH₊), measured 455 (MH₊). ¹H NMR (400 MHz, METHANOL-d₄) δ=9.00 (dd, J=1.6, 4.2 Hz, 1H), 8.66 (dd, J=1.7, 8.6 Hz, 1H), 8.15 (dd, J=7.6, 17.1 Hz, 2H), 7.66 (dd, J=4.3, 8.6 Hz, 1H), 7.29 (d, J=8.1 Hz, 1H), 7.19 (br s, 1H), 6.62 (br s, 1H), 5.23 (br s, 2H), 4.55 (br d, J=12.7 Hz, 2H), 4.37 (br t, J=9.8 Hz, 1H), 4.22-4.04 (m, 3H), 3.73-3.54 (m, 2H), 3.44 (br t, J=11.4 Hz, 2H), 2.98 (t, J=7.5 Hz, 2H), 2.87-2.68 (m, 2H), 2.60 (br s, 3H), 1.31 (d, J=6.2 Hz, 3H).

### Example 16

### 5-[2R,6S)-2-methyl-6-[[8-(2-methyl-4-pyridyl)-5-oxa-2,8-diazaspiro[3.5]nonan-2-yl]methyl]morpholin-4-yl]quinoline-8-carbonitrile

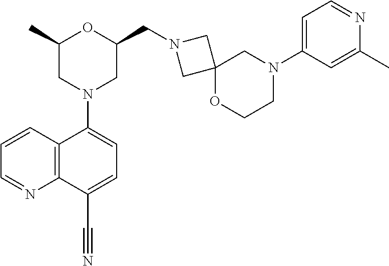 [SMILES: Cc1cc(N2CCOC3(CN(C[C@H]4CN(c5ccc(C#N)c6ncccc56)C[C@@H](C)O4)C3)C2)ccn1]

The title compound was prepared in analogy to the preparation of Example 1 by using 4-bromo-2-methyl-pyridine and tert-butyl 5-oxa-2,8-diazaspiro[3.5]nonane-2-carboxylate instead of (4-bromo-6-methyl-2-pyridyl)methanol (compound 1a) and tert-butyl 2,6-diazaspiro[3.3]heptane-2-carboxylate;oxalic acid (compound 1b). Example 16 (35 mg) was obtained as a light yellow solid. MS: calc'd 485 (MH₊), measured 485 (MH₊). ¹H NMR (400 MHz, METHANOL-d₄) δ=8.98 (br d, J=3.1 Hz, 1H), 8.63 (br d, J=8.3 Hz, 1H), 8.13 (br t, J=6.8 Hz, 2H), 7.64 (br dd, J=4.2, 8.4 Hz, 1H), 7.25 (br d, J=7.9 Hz, 1H), 7.22-7.09 (m, 2H), 4.46 (br s, 2H), 4.35 (br s, 3H), 4.19-4.01 (m, 3H), 3.96 (br s, 2H), 3.71 (br s, 2H), 3.65-3.57 (m, 1H), 3.57-3.46 (m, 1H), 3.40 (br d, J=12.0 Hz, 2H), 2.87-2.64 (m, 2H), 2.58 (s, 3H), 1.28 (br d, J=6.1 Hz, 3H).

### Example 17, 17A and 17B

### 5-[2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile, trans-5-[2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile and cis-5-[2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile

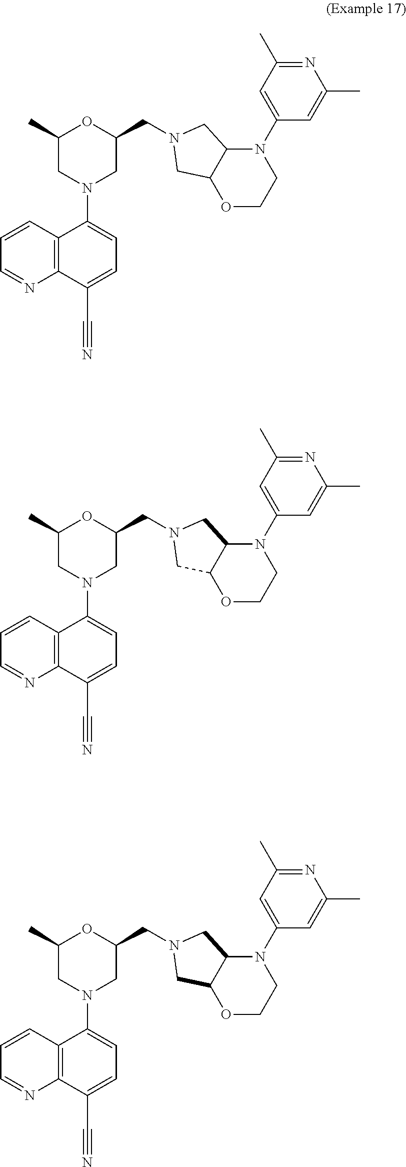

Example 17 was prepared in analogy to the preparation of Example 1 by using 4-bromo-2,6-dimethyl-pyridine and tert-butyl 3,4,4a,5,7,7a-hexahydro-2H-pyrrolo[3,4-b][1,4]oxazine-6-carboxylate (CAS: 1360364-21-1, Vendor: PharmaBlock) instead of (4-bromo-6-methyl-2-pyridyl)methanol (compound 1a) and tert-butyl 2,6-diazaspiro[3.3]heptane-2-carboxylate;oxalic acid (compound 1b). The trans and cis isomers were obtained by flash column separation (EA/PE=0 to 100%).

Example 17A (38 mg) was obtained as a light yellow solid. MS: calc'd 499 (MH₊), measured 499 (MH₊). ¹H NMR (400 MHz, METHANOL-d₄) δ=8.99 (br s, 1H), 8.66 (br t, J=6.8 Hz, 1H), 8.16 (d, J=8.1 Hz, 1H), 7.64 (td, J=4.1, 8.3 Hz, 1H), 7.26 (br d, J=7.9 Hz, 1H), 6.66 (s, 2H), 4.09 (br d, J=11.6 Hz, 3H), 3.98-3.88 (m, 1H), 3.88-3.78 (m, 1H), 3.70-3.52 (m, 2H), 3.49-3.37 (m, 2H), 3.22-3.10 (m, 2H), 3.04-2.79 (m, 5H), 2.79-2.60 (m, 2H), 2.42 (s, 6H), 1.27 (br d, J=4.8 Hz, 3H).

Example 17B (27 mg) was obtained as a light yellow solid. MS: calc'd 499 (MH₊), measured 499 (MH₊). ¹H NMR (400 MHz, METHANOL-d₄) δ=8.98 (br d, J=1.7 Hz, 1H), 8.64 (br t, J=6.7 Hz, 1H), 8.14 (d, J=7.9 Hz, 1H), 7.62 (td, J=4.2, 8.5 Hz, 1H), 7.25 (d, J=7.9 Hz, 1H), 6.58 (s, 2H), 4.35-4.23 (m, 1H), 4.19-3.97 (m, 4H), 3.65 (br t, J=11.2 Hz, 1H), 3.56-3.28 (m, 4H), 3.19-3.03 (m, 2H), 2.89-2.59 (m, 6H), 2.39 (d, J=1.7 Hz, 6H), 1.26 (dd, J=1.9, 6.2 Hz, 3H).

### Example 19

### 5-[(2S,6R)-2-[[4-(2-amino-4-pyridyl)piperazin-1-yl]methyl]-6-methyl-morpholin-4-yl]quinoline-8-carbonitrile

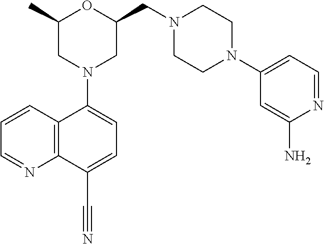 [SMILES: C[C@@H]1CN(c2ccc(C#N)c3ncccc23)C[C@H](CN2CCN(c3ccnc(N)c3)CC2)O1]

The title compound was prepared according to the following scheme:

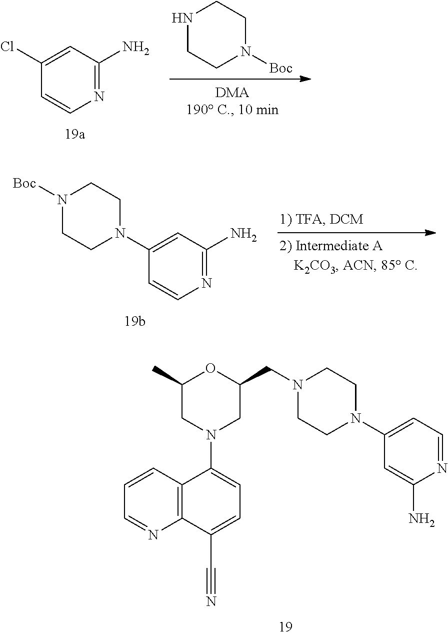 [SMILES: Nc1cc(Cl)ccn1 ; CC(C)(C)OC(=O)N1CCNCC1 ; CC(C)(C)OC(=O)N1CCN(c2ccnc(N)c2)CC1 ; C[C@@H]1CN(c2ccc(C#N)c3ncccc23)C[C@H](CN2CCN(c3ccnc(N)c3)CC2)O1]

Step 1: preparation of tert-butyl 4-(2-amino-4-pyridyl)piperazine-1-carboxylate (compound 19b)

A mixture of 4-chloropyridin-2-amine (compound 19a, CAS: 19798-80-2, Vendor: Aldrich, 129 mg, 1.00 mmol) and tert-butyl piperazine-1-carboxylate (CAS: 57260-71-6, Vendor: Accela ChemBio Inc, 186 mg, 1.00 mmol) in N,N-dimethylacetamide (3 mL) was heated at 190° C. for 10 minutes. After the reaction mixture was cooled down, the solid was collected by filtration to give compound 19b (223 mg) as a grey solid. MS: calc'd 279 (MH₊), measured 279 (MH₊).

Step 2: preparation of 5-[(2S,6R)-2-[[4-(2-amino-4-pyridyl)piperazin-1-yl]methyl]-6-methyl-morpholin-4-yl]quinoline-8-carbonitrile (Example 19)

The mixture of tert-butyl 4-(2-amino-4-pyridyl)piperazine-1-carboxylate (compound 19b, 67 mg, 240 μmol) and DCM/TFA=1/2 (3 mL) was stirred at room temperature for 1 h, then it was concentrated and the residue was dissolved in ACN (6 mL), to which K₂CO₃ (83 mg, 600 μmol) and [2R,6R)-4-(8-cyano-5-quinolyl)-6-methyl-morpholin-2-yl]methyl trifluoromethanesulfonate (Intermediate A, 83 mg, 200 μmol) were added. After being stirred at 85° C. for 2 hrs, the reaction mixture was filtered and then directly purified by prep-HPLC to give Example 19 (16 mg). MS: calc'd 444 (MH₊), measured 444 (MH₊). ¹H NMR (400 MHz, METHANOL-d₄) δ=9.01 (dd, J=1.6, 4.3 Hz, 1H), 8.68 (dd, J=1.7, 8.6 Hz, 1H), 8.18 (d, J=7.9 Hz, 1H), 7.67 (dd, J=4.3, 8.6 Hz, 1H), 7.64 (d, J=7.5 Hz, 1H), 7.30 (d, J=7.9 Hz, 1H), 6.66 (dd, J=2.6, 7.6 Hz, 1H), 6.17 (d, J=2.4 Hz, 1H), 4.58-4.48 (m, 1H), 4.27-4.15 (m, 1H), 4.09-3.75 (m, 4H), 3.68-3.49 (m, 4H), 3.48-3.39 (m, 4H), 2.84-2.71 (m, 2H), 1.33 (d, J=6.2 Hz, 3H).

### Example 20

### 5-[(2S,6R)-2-[[[1-(2-amino-4-pyridyl)-4-piperidyl]amino]methyl]-6-methyl-morpholin-4-yl]quinoline-8-carbonitrile

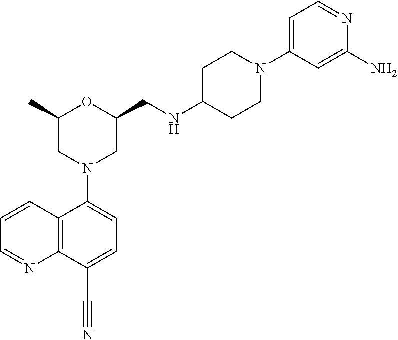 [SMILES: C[C@@H]1CN(c2ccc(C#N)c3ncccc23)C[C@H](CNC2CCN(c3ccnc(N)c3)CC2)O1]

The title compound was prepared according to the following scheme:

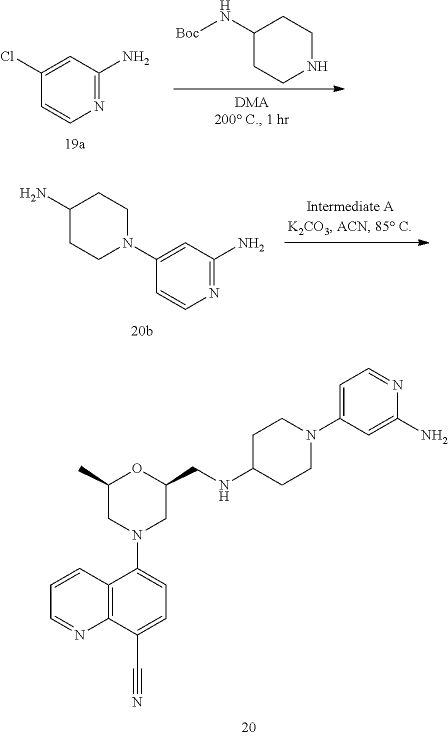 [SMILES: Nc1cc(Cl)ccn1 ; CC(C)(C)OC(=O)NC1CCNCC1 ; Nc1cc(N2CCC(N)CC2)ccn1 ; C[C@@H]1CN(c2ccc(C#N)c3ncccc23)C[C@H](CNC2CCN(c3ccnc(N)c3)CC2)O1]

Step 1: preparation of 4-(4-amino-1-piperidyl)pyridin-2-amine (compound 20b)

A mixture of 4-chloropyridin-2-amine (129 mg, 1.00 mmol) and tert-butyl piperidin-4-ylcarbamate (CAS: 73874-95-0, Vendor: Accela ChemBio Inc, 200 mg, 1.00 mmol) in N,N-dimethylacetamide (3 mL) was stirred at 200° C. for 1 h. After the reaction mixture was cooled down, the solution was added dropwise to methyl tert-butyl ether (60 mL). The solid was collected by centrifugation, then dissolved in MeOH (5 mL). To this solution was added 5 drops of 5 M NaOMe solution in methanol. After being stirred for 5 minutes, the reaction mixture was added with solid NaHCO₃ (500 mg) and stirred for another 15 minutes, then EA (15 mL) was added. The mixture was filtered and the organic phase was concentrated to give crude compound 20b (75 mg) which was used directly for next step. MS: calc'd 193 (MH₊), measured 193 (MH₊).

Step 2: preparation of 5-[(2S,6R)-2-[[[1-(2-amino-4-pyridyl)-4-piperidyl]amino]methyl]-6-methyl-morpholin-4-yl]quinoline-8-carbonitrile (Example 20)

To a mixture of [(2R,6R)-4-(8-cyano-5-quinolyl)-6-methyl-morpholin-2-yl]methyl trifluoromethanesulfonate (Intermediate A, 50 mg, 120 μmol) and 4-(4-aminopiperidin-1-yl)pyridin-2-amine (compound 20b, 33 mg, 170 μmol) in ACN (6 mL) was added K₂CO₃ (50 mg, 361 μmol). After being stirred at 85° C. overnight, the reaction mixture was filtered and the organic phase was directly purified by prep-HPLC to give Example 20 (25 mg). MS: calc'd 458 (MH₊), measured 458 (MH₊). ¹H NMR (400 MHz, METHANOL-d₄) δ=9.00 (dd, J=1.5, 4.2 Hz, 1H), 8.65 (dd, J=1.6, 8.6 Hz, 1H), 8.16 (d, J=7.9 Hz, 1H), 7.66 (dd, J=4.3, 8.6 Hz, 1H), 7.56 (d, J=7.6 Hz, 1H), 7.28 (d, J=8.1 Hz, 1H), 6.62 (dd, J=2.6, 7.6 Hz, 1H), 6.11 (d, J=2.6 Hz, 1H), 4.40-4.28 (m, 1H), 4.28-4.12 (m, 3H), 3.58 (ddd, J=4.5, 7.3, 11.6 Hz, 1H), 3.50-3.35 (m, 3H), 3.28-3.12 (m, 3H), 2.86-2.68 (m, 2H), 2.36-2.25 (m, 2H), 1.73 (dq, J=4.1, 12.2 Hz, 2H), 1.32 (d, J=6.4 Hz, 3H).

### Example 21

### 5-[(2S,6R)-2-[[[(3R)-1-(2-amino-4-pyridyl)-3-piperidyl]amino]methyl]-6-methyl-morpholin-4-yl]quinoline-8-carbonitrile

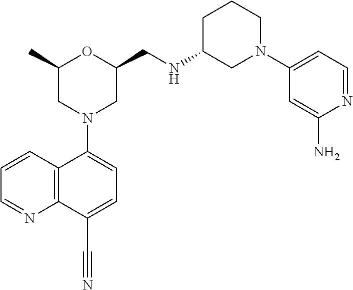 [SMILES: C[C@@H]1CN(c2ccc(C#N)c3ncccc23)C[C@H](CN[C@@H]2CCCN(c3ccnc(N)c3)C2)O1]

The title compound was prepared in analogy to the preparation of Example 20 by using tert-butyl N-[(3R)-3-piperidyl]carbamate (CAS: 309956-78-3, Vendor: TCI) instead of tert-butyl piperidin-4-ylcarbamate. Example 21 (8 mg) was obtained. MS: calc'd 458 (MH₊), measured 458 (MH₊). ¹H NMR (400 MHz, METHANOL-d₄) δ=9.01 (dd, J=1.6, 4.3 Hz, 1H), 8.66 (dd, J=1.7, 8.6 Hz, 1H), 8.18 (d, J=8.1 Hz, 1H), 7.67 (dd, J=4.2, 8.6 Hz, 1H), 7.58 (d, J=7.6 Hz, 1H), 7.30 (d, J=8.1 Hz, 1H), 6.63 (dd, J=2.7, 7.7 Hz, 1H), 6.17 (d, J=2.6 Hz, 1H), 4.40-4.31 (m, 2H), 4.22-4.13 (m, 1H), 3.98 (br d, J=13.6 Hz, 1H), 3.51-3.37 (m, 4H), 3.35 (s, 1H), 3.31-3.27 (m, 1H), 3.25-3.14 (m, 1H), 2.87-2.70 (m, 2H), 2.40-2.32 (m, 1H), 2.06-1.94 (m, 1H), 1.88-1.66 (m, 2H), 1.33 (d, J=6.4 Hz, 3H).

### Example 22

### 5-[2S,6R)-2-[[[(3S)-1-(2-amino-4-pyridyl)-3-piperidyl]amino]methyl]-6-methyl-morpholin-4-yl]quinoline-8-carbonitrile

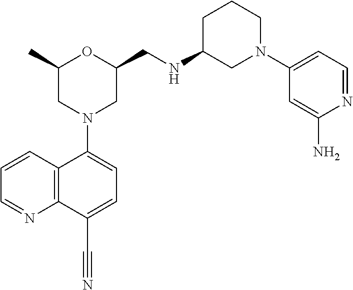 [SMILES: C[C@@H]1CN(c2ccc(C#N)c3ncccc23)C[C@H](CN[C@H]2CCCN(c3ccnc(N)c3)C2)O1]

The title compound was prepared in analogy to the preparation of Example 20 by using tert-butyl N-[(3S)-3-piperidyl]carbamate (CAS: 216854-23-8, Vendor: TCI) instead of tert-butyl piperidin-4-ylcarbamate. Example 22 (15 mg) was obtained. MS: calc'd 458 (MH₊), measured 458 (MH₊). ¹H NMR (400 MHz, METHANOL-d₄) δ=9.01 (dd, J=1.6, 4.3 Hz, 1H), 8.66 (dd, J=1.7, 8.6 Hz, 1H), 8.18 (d, J=8.1 Hz, 1H), 7.66 (dd, J=4.3, 8.6 Hz, 1H), 7.58 (d, J=7.6 Hz, 1H), 7.29 (d, J=8.1 Hz, 1H), 6.64 (dd, J=2.6, 7.6 Hz, 1H), 6.17 (d, J=2.6 Hz, 1H), 4.40-4.28 (m, 2H), 4.25-4.12 (m, 1H), 3.95 (br d, J=13.3 Hz, 1H), 3.52-3.40 (m, 4H), 3.39-3.35 (m, 1H), 3.30-3.14 (m, 2H), 2.88-2.68 (m, 2H), 2.42-2.29 (m, 1H), 2.07-1.95 (m, 1H), 1.90-1.67 (m, 2H), 1.33 (d, J=6.2 Hz, 3H).

### Example 23

5-[2S,6R)-2-[[[1-[2-(hydroxymethyl)-6-methyl-4-pyridyl]-4-piperidyl]amino]methyl]-6-methyl-morpholin-4-yl]quinoline-8-carbonitrile

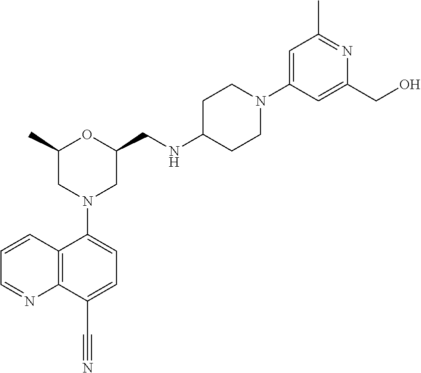 [SMILES: Cc1cc(N2CCC(NC[C@H]3CN(c4ccc(C#N)c5ncccc45)C[C@@H](C)O3)CC2)cc(CO)n1]

The title compound was prepared in analogy to the preparation of Example 1 by using tert-butyl piperidin-4-ylcarbamate instead of tert-butyl 2,6-diazaspiro[3.3]heptane-2-carboxylate;oxalic acid (compound 1b). Example 23 (3 mg) was obtained. MS: calc'd 487 (MH₊), measured 487 (MH₊). ¹H NMR (400 MHz, METHANOL-d₄) δ=9.00 (d, J=3.1 Hz, 1H), 8.67 (br d, J=8.2 Hz, 1H), 8.17 (d, J=8.1 Hz, 1H), 7.66 (dd, J=4.2, 8.4 Hz, 1H), 7.28 (d, J=7.9 Hz, 1H), 7.03 (s, 1H), 6.97 (s, 1H), 4.68 (s, 2H), 4.35 (br d, J=13.4 Hz, 2H), 4.30-4.22 (m, 1H), 4.22-4.11 (m, 1H), 3.51-3.35 (m, 3H), 3.31-3.06 (m, 4H), 2.83-2.68 (m, 2H), 2.54 (s, 3H), 2.27 (br s, 2H), 1.66 (br s, 2H), 1.31 (d, J=6.2 Hz, 3H).

### Example 24

5-[2S,6R)-2-[[[(3R)-1-[2-(hydroxymethyl)-6-methyl-4-pyridyl]-3-piperidyl]amino]methyl]-6-methyl-morpholin-4-yl]quinoline-8-carbonitrile

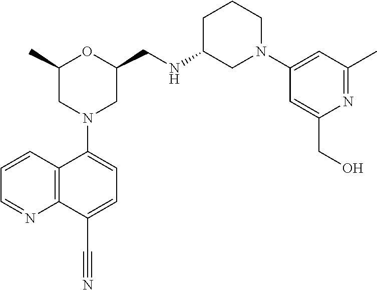 [SMILES: Cc1cc(N2CCC[C@@H](NC[C@H]3CN(c4ccc(C#N)c5ncccc45)C[C@@H](C)O3)C2)cc(CO)n1]

The title compound was prepared in analogy to the preparation of Example 1 by using tert-butyl N-[(3R)-3-piperidyl]carbamate instead of tert-butyl 2,6-diazaspiro[3.3]heptane-2-carboxylate;oxalic acid (compound 1b). Example 24 (8 mg) was obtained. MS: calc'd 487 (MH₊), measured 487 (MH₊). ¹H NMR (400 MHz, METHANOL-d₄) δ=8.98 (d, J=2.9 Hz, 1H), 8.64 (d, J=7.6 Hz, 1H), 8.14 (d, J=7.9 Hz, 1H), 7.64 (dd, J=4.3, 8.6 Hz, 1H), 7.24 (d, J=8.1 Hz, 1H), 7.03 (s, 1H), 6.96 (s, 1H), 4.67 (s, 2H), 4.34 (br d, J=12.5 Hz, 1H), 4.24 (br s, 1H), 4.18-4.05 (m, 2H), 3.44 (br dd, J=11.9, 18.6 Hz, 2H), 3.37-3.33 (m, 1H), 3.31-3.26 (m, 1H), 3.19-3.06 (m, 3H), 2.83-2.66 (m, 2H), 2.53 (s, 3H), 2.38-2.21 (m, 1H), 1.99 (br d, J=13.2 Hz, 1H), 1.81-1.61 (m, 2H), 1.30 (d, J=6.2 Hz, 3H).

### Example 25

### 5-[2S,6R)-2-[[[(3S)-1-[2-(hydroxymethyl)-6-methyl-4-pyridyl]-3-piperidyl]amino]methyl]-6-methyl-morpholin-4-yl]quinoline-8-carbonitrile

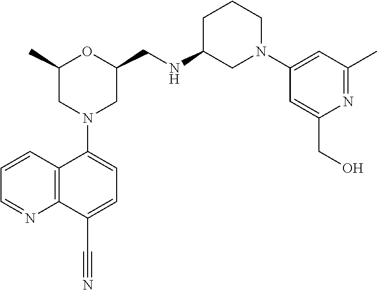 [SMILES: Cc1cc(N2CCC[C@H](NC[C@H]3CN(c4ccc(C#N)c5ncccc45)C[C@@H](C)O3)C2)cc(CO)n1]

The title compound was prepared in analogy to the preparation of Example 1 by using tert-butyl N-[(3S)-3-piperidyl]carbamate instead of tert-butyl 2,6-diazaspiro[3.3]heptane-2-carboxylate;oxalic acid (compound 1b). Example 25 (1 mg) was obtained. MS: calc'd 487 (MH₊), measured 487 (MH₊). ¹H NMR (400 MHz, METHANOL-d₄) δ=9.00 (dd, J=1.5, 4.2 Hz, 1H), 8.66 (dd, J=1.6, 8.6 Hz, 1H), 8.17 (d, J=7.9 Hz, 1H), 7.65 (dd, J=4.3, 8.6 Hz, 1H), 7.26 (d, J=8.1 Hz, 1H), 7.03 (d, J=2.3 Hz, 1H), 6.95 (d, J=2.2 Hz, 1H), 4.70-4.65 (m, 2H), 4.26 (br d, J=12.6 Hz, 1H), 4.19-4.02 (m, 3H), 3.43 (br t, J=10.0 Hz, 2H), 3.37-3.34 (m, 1H), 3.31-3.23 (m, 1H), 3.06-2.90 (m, 3H), 2.83-2.74 (m, 1H), 2.74-2.65 (m, 1H), 2.57-2.47 (m, 3H), 2.27-2.15 (m, 1H), 2.01-1.90 (m, 1H), 1.73-1.59 (m, 2H), 1.28 (d, J=6.2 Hz, 3H).

### Example 26

### trans-5-[2S,6R)-2-[[[3-hydroxy-1-[2-(hydroxymethyl)-6-methyl-4-pyridyl]-4-piperidyl]amino]methyl]-6-methyl-morpholin-4-yl]quinoline-8-carbonitrile

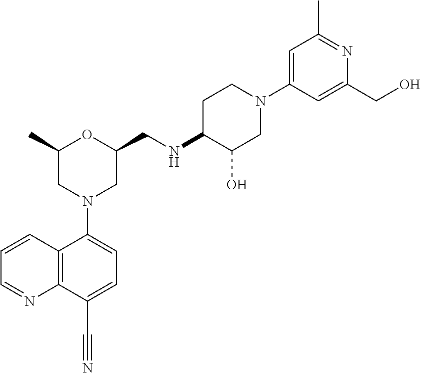 [SMILES: Cc1cc(N2CC[C@H](NC[C@H]3CN(c4ccc(C#N)c5ncccc45)C[C@@H](C)O3)[C@@H](O)C2)cc(CO)n1]

The title compound was prepared according to the following scheme:

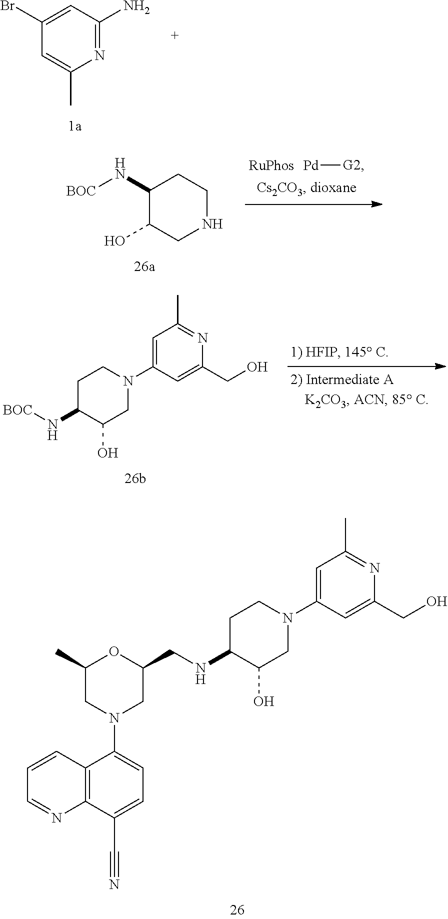

Step 1: preparation of trans-tert-butyl N-[3-hydroxy-1-[2-(hydroxymethyl)-6-methyl-4-pyridyl]-4-piperidyl]carbamate (compound 26b)

The mixture of (4-bromo-6-methyl-2-pyridyl)methanol (compound 1a, 152 mg, 750 μmol), trans-tert-butyl(3-hydroxypiperidin-4-yl)carbamate (CAS: 859854-66-3, Vendor: PharmaBlock, 108 mg, 500 μmol) and Cs₂CO₃ (326 mg, 1.00 mmol) in 1,4-dioxane (5 mL) was bubbled with N₂ for 5 mins and Ruphos Pd G2 (12 mg, 15 μmol) was added. Then the mixture was sealed and stirred at 85° C. for 4 hrs. After being cooled down, the mixture was diluted with EA (20 mL), filtered and concentrated, the residue was purified by flash column (MeOH/DCM=10 to 20%) to give compound 26b (110 mg). MS: calc'd 338 (MH₊), measured 338 (MH₊).

Step 2: preparation of trans-5-[2S,6R)-2-[[[3-hydroxy-1-[2-(hydroxymethyl)-6-methyl-4-pyridyl]-4-piperidyl]amino]methyl]-6-methyl-morpholin-4-yl]quinoline-8-carbonitrile (Example 26)

The mixture of trans-tert-butyl N-[3-hydroxy-1-[2-(hydroxymethyl)-6-methyl-4-pyridyl]-4-piperidyl]carbamate (compound 26b, 57 mg, 169 μmol) and 1,1,1,3,3,3-hexafluoro-2-propanol (10 mL) was stirred at 145° C. for 40 minutes under microwave. The reaction mixture was concentrated and the residue was dissolved in dry ACN (5 mL) followed by adding [(2R,6R)-4-(8-cyano-5-quinolyl)-6-methyl-morpholin-2-yl]methyl trifluoromethanesulfonate (Intermediate A, 50 mg, 120 μmol) and K₂CO₃ (50 mg, 361 μmol). After being stirred at 85° C. for 4 hrs, the reaction mixture was filtered and the organic phase was directly purified by prep-HPLC to give Example 26 (22 mg). MS: calc'd 503 (MH₊), measured 503 (MH₊). ¹H NMR (400 MHz, METHANOL-d₄) δ=9.00 (dd, J=1.5, 4.2 Hz, 1H), 8.69-8.63 (m, 1H), 8.17 (d, J=7.9 Hz, 1H), 7.66 (dd, J=4.3, 8.6 Hz, 1H), 7.29 (d, J=7.9 Hz, 1H), 7.08 (s, 1H), 7.02 (s, 1H), 4.71 (s, 2H), 4.46-4.32 (m, 3H), 4.23-4.13 (m, 1H), 3.83 (dq, J=4.8, 9.7 Hz, 1H), 3.54-3.40 (m, 4H), 3.40-3.34 (m, 1H), 3.30-3.23 (m, 1H), 3.18-3.09 (m, 1H), 2.86-2.69 (m, 2H), 2.56 (s, 3H), 2.46-2.33 (m, 1H), 1.80 (dq, J=4.0, 12.6 Hz, 1H), 1.32 (dd, J=2.2, 6.2 Hz, 3H).

### Example 27

### trans-5-[2S,6R)-2-[[[4-hydroxy-1-[2-(hydroxymethyl)-6-methyl-4-pyridyl]-3-piperidyl]amino]methyl]-6-methyl-morpholin-4-yl]quinoline-8-carbonitrile

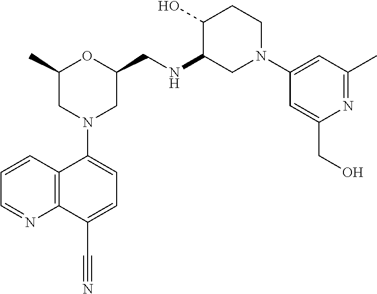 [SMILES: Cc1cc(N2CC[C@@H](O)[C@H](NC[C@H]3CN(c4ccc(C#N)c5ncccc45)C[C@@H](C)O3)C2)cc(CO)n1]

The title compound was prepared in analogy to the preparation of Example 26 by using trans-tert-butyl N-(4-hydroxy-3-piperidyl)carbamate (CAS: 859854-68-5, Vendor: PharmaBlock) instead of trans-tert-butyl (3-hydroxypiperidin-4-yl)carbamate (compound 26a). Example 27 (19 mg) was obtained. MS: calc'd 503 (MH₊), measured 503 (MH₊). ¹H NMR (400 MHz, METHANOL-d₄) δ=9.02-8.99 (m, 1H), 8.66 (td, J=2.0, 8.6 Hz, 1H), 8.17 (d, J=8.1 Hz, 1H), 7.66 (dd, J=4.2, 8.6 Hz, 1H), 7.29 (d, J=8.1 Hz, 1H), 7.15-7.12 (m, 1H), 7.07 (d, J=7.9 Hz, 1H), 4.72 (s, 2H), 4.68-4.60 (m, 1H), 4.46-4.34 (m, 1H), 4.34-4.26 (m, 1H), 4.24-4.13 (m, 1H), 4.13-4.03 (m, 1H), 3.61-3.34 (m, 6H), 3.30-3.25 (m, 1H), 2.88-2.70 (m, 2H), 2.57 (d, J=1.5 Hz, 3H), 2.30-2.18 (m, 1H), 1.76-1.63 (m, 1H), 1.32 (dd, J=6.4, 10.0 Hz, 3H).

### Example 28

### 5-[2S,6R)-2-[[[(3R)-1-[2-(hydroxymethyl)-6-methyl-4-pyridyl]pyrrolidin-3-yf]amino]methyl]-6-methyl-morpholin-4-yl]quinoline-8-carbonitrile

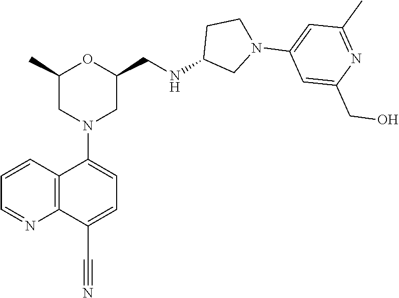 [SMILES: Cc1cc(N2CC[C@@H](NC[C@H]3CN(c4ccc(C#N)c5ncccc45)C[C@@H](C)O3)C2)cc(CO)n1]

The title compound was prepared in analogy to the preparation of Example 26 by using tert-butyl N-[(3R)-pyrrolidin-3-yl]carbamate (CAS: 122536-77-0, Vendor: Accela ChemBio Inc) instead of trans-tert-butyl (3-hydroxypiperidin-4-yl)carbamate (compound 26a). Example 28 (15 mg) was obtained. MS: calc'd 473 (MH₊), measured 473 (MH₊). ¹H NMR (400 MHz, METHANOL-d₄) δ=9.01 (dd, J=1.7, 4.2 Hz, 1H), 8.66 (dd, J=1.6, 8.6 Hz, 1H), 8.18 (d, J=7.9 Hz, 1H), 7.66 (dd, J=4.2, 8.6 Hz, 1H), 7.29 (d, J=8.1 Hz, 1H), 6.79 (br d, J=5.4 Hz, 1H), 6.76-6.66 (m, 1H), 4.71 (s, 2H), 4.37 (br t, J=10.0 Hz, 1H), 4.26-4.13 (m, 2H), 4.08-3.98 (m, 1H), 3.92-3.80 (m, 2H), 3.80-3.61 (m, 1H), 3.52-3.40 (m, 3H), 3.31-3.26 (m, 1H), 2.86-2.73 (m, 2H), 2.72-2.61 (m, 1H), 2.57 (s, 3H), 2.51-2.39 (m, 1H), 1.33 (d, J=6.2 Hz, 3H).

### Example 29

### 5-[2S,6R)-2-[[[(3S)-1-[2-(hydroxymethyl)-6-methyl-4-pyridyl]pyrrolidin-3-yl]amino]methyl]-6-methyl-morpholin-4-yl]quinoline-8-carbonitrile

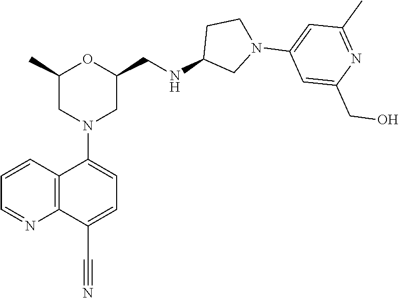 [SMILES: Cc1cc(N2CC[C@H](NC[C@H]3CN(c4ccc(C#N)c5ncccc45)C[C@@H](C)O3)C2)cc(CO)n1]

The title compound was prepared in analogy to the preparation of Example 26 by using tert-butyl N-[(3S)-pyrrolidin-3-yl]carbamate (CAS: 122536-76-9, Vendor: Accela ChemBio Inc) instead of trans-tert-butyl (3-hydroxypiperidin-4-yl)carbamate (compound 26a). Example 29 (17 mg) was obtained. MS: calc'd 473 (MH₊), measured 473 (MH₊). ¹H NMR (400 MHz, METHANOL-d₄) δ=9.02 (dd, J=1.6, 4.3 Hz, 1H), 8.67 (dd, J=1.7, 8.6 Hz, 1H), 8.19 (d, J=8.1 Hz, 1H), 7.67 (dd, J=4.2, 8.6 Hz, 1H), 7.31 (d, J=8.1 Hz, 1H), 6.80 (br d, J=6.1 Hz, 1H), 6.71 (br d, J=14.4 Hz, 1H), 4.72 (s, 2H), 4.36 (br t, J=10.2 Hz, 1H), 4.26-4.14 (m, 2H), 4.07 (br d, J=10.4 Hz, 1H), 3.91-3.78 (m, 2H), 3.78-3.66 (m, 1H), 3.54-3.42 (m, 3H), 3.31-3.26 (m, 1H), 2.87-2.72 (m, 2H), 2.66 (dt, J=6.6, 13.8 Hz, 1H), 2.58 (s, 3H), 2.49-2.38 (m, 1H), 1.33 (d, J=6.2 Hz, 3H).

### Example 30

### 5-[2S,6R)-2-[[[1-[2-(hydroxymethyl)-6-methyl-4-pyridyl]azetidin-3-yl]amino]methyl]-6-methyl-morpholin-4-yl]quinoline-8-carbonitrile

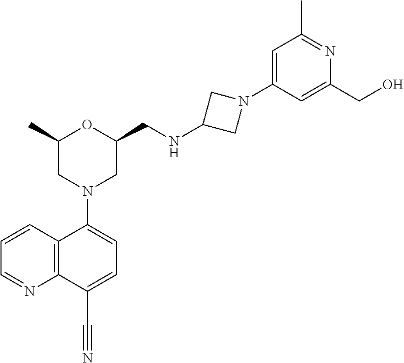 [SMILES: Cc1cc(N2CC(NC[C@H]3CN(c4ccc(C#N)c5ncccc45)C[C@@H](C)O3)C2)cc(CO)n1]

The title compound was prepared in analogy to the preparation of Example 26 by using tert-butyl N-(azetidin-3-yl)carbamate (CAS: 91188-13-5, Vendor: PharmaBlock) instead of trans-tert-butyl (3-hydroxypiperidin-4-yl)carbamate (compound 26a). Example 30 (7 mg) was obtained. MS: calc'd 459 (MH₊), measured 459 (MH₊). ¹H NMR (400 MHz, METHANOL-d₄) δ=9.02 (dd, J=1.6, 4.3 Hz, 1H), 8.68 (dd, J=1.7, 8.6 Hz, 1H), 8.19 (d, J=8.1 Hz, 1H), 7.67 (dd, J=4.3, 8.6 Hz, 1H), 7.30 (d, J=7.9 Hz, 1H), 6.62 (d, J=2.2 Hz, 1H), 6.53 (d, J=2.0 Hz, 1H), 4.69 (s, 2H), 4.67-4.58 (m, 2H), 4.50-4.39 (m, 3H), 4.34 (br t, J=9.8 Hz, 1H), 4.25-4.14 (m, 1H), 3.51-3.42 (m, 2H), 3.40-3.35 (m, 1H), 3.23 (dd, J=9.6, 12.9 Hz, 1H), 2.86-2.71 (m, 2H), 2.55 (s, 3H), 1.34 (d, J=6.2 Hz, 3H).

### Example 31

### trans-5-[2S,6R)-2-[[[4-hydroxy-1-[2-(hydroxymethyl)-6-methyl-4-pyridyl]pyrrolidin-3-yl]amino]methyl]-6-methyl-morpholin-4-yl]quinoline-8-carbonitrile

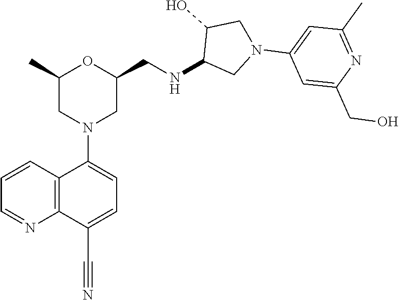 [SMILES: Cc1cc(N2C[C@@H](O)[C@H](NC[C@H]3CN(c4ccc(C#N)c5ncccc45)C[C@@H](C)O3)C2)cc(CO)n1]

The title compound was prepared in analogy to the preparation of Example 26 by using trans-tert-butyl N-(4-hydroxypyrrolidin-3-yl)carbamate (CAS: 870632-89-6, Vendor: PharmaBlock) instead of trans-tert-butyl (3-hydroxypiperidin-4-yl)carbamate (compound 26a). Example 31 (6 mg) was obtained. MS: calc'd 489 (MH₊), measured 489 (MH₊). ¹H NMR (400 MHz, METHANOL-d₄) δ=9.02 (td, J=1.6, 4.2 Hz, 1H), 8.67 (ddd, J=1.7, 6.8, 8.5 Hz, 1H), 8.19 (dd, J=1.3, 8.0 Hz, 1H), 7.67 (td, J=4.1, 8.4 Hz, 1H), 7.30 (dd, J=1.5, 8.0 Hz, 1H), 6.80 (s, 1H), 6.72 (br s, 1H), 4.77-4.71 (m, 3H), 4.40-4.32 (m, 1H), 4.20-3.92 (m, 4H), 3.88-3.75 (m, 1H), 3.59-3.42 (m, 4H), 3.40-3.35 (m, 1H), 2.85-2.72 (m, 2H), 2.58 (s, 3H), 1.33 (dd, J=2.2, 6.2 Hz, 3H).

### Example 32

### trans-5-[2S,6R)-2-[[[1-(2,6-dimethyl-4-pyridyl)-4-hydroxy-3-piperidyl]amino]methyl]-6-methyl-morpholin-4-yl]quinoline-8-carbonitrile

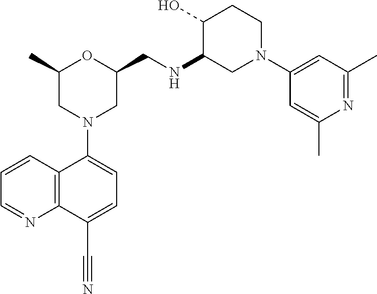 [SMILES: Cc1cc(N2CC[C@@H](O)[C@H](NC[C@H]3CN(c4ccc(C#N)c5ncccc45)C[C@@H](C)O3)C2)cc(C)n1]

The title compound was prepared in analogy to the preparation of Example 26 by using trans-tert-butyl N-(4-hydroxy-3-piperidyl)carbamate (CAS: 859854-68-5, Vendor: PharmaBlock) and 4-bromo-2,6-dimethyl-pyridine instead of trans-tert-butyl (3-hydroxypiperidin-4-yl)carbamate (compound 26a) and (4-bromo-6-methyl-2-pyridyl)methanol (compound 1a). Example 32 (8 mg) was obtained. MS: calc'd 487 (MH₊), measured 487 (MH₊). ¹H NMR (400 MHz, METHANOL-d₄) δ=9.02 (dd, J=1.6, 4.3 Hz, 1H), 8.67 (ddd, J=1.7, 3.1, 8.6 Hz, 1H), 8.19 (d, J=7.9 Hz, 1H), 7.67 (dd, J=4.3, 8.6 Hz, 1H), 7.31 (d, J=8.1 Hz, 1H), 7.02 (d, J=8.6 Hz, 2H), 4.60 (br d, J=11.7 Hz, 1H), 4.44-4.26 (m, 2H), 4.26-4.14 (m, 1H), 4.14-4.00 (m, 1H), 3.57-3.40 (m, 4H), 3.38-3.35 (m, 1H), 3.31-3.20 (m, 2H), 2.89-2.70 (m, 2H), 2.54 (d, J=2.0 Hz, 6H), 2.30-2.19 (m, 1H), 1.75-1.62 (m, 1H), 1.32 (dd, J=6.2, 12.6 Hz, 3H).

### Example 33

### cis-5-[2S,6R)-2-[[[4-fluoro-1-[2-(hydroxymethyl)-6-methyl-4-pyridyl]-3-piperidyl]amino]methyl]-6-methyl-morpholin-4-yl]quinoline-8-carbonitrile

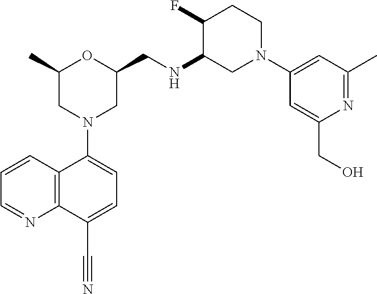 [SMILES: Cc1cc(N2CC[C@H](F)[C@H](NC[C@H]3CN(c4ccc(C#N)c5ncccc45)C[C@@H](C)O3)C2)cc(CO)n1]

The title compound was prepared in analogy to the preparation of Example 26 by using cis-tert-butyl N-(4-fluoro-3-piperidyl)carbamate (CAS: 1363382-99-3, Vendor: PharmaBlock) instead of trans-tert-butyl (3-hydroxypiperidin-4-yl)carbamate (compound 26a). Example 33 (9 mg) was obtained. MS: calc'd 505 (MH₊), measured 505 (MH₊). ¹H NMR (400 MHz, METHANOL-d₄) δ=9.02 (dd, J=1.6, 4.3 Hz, 1H), 8.68 (d, J=8.6 Hz, 1H), 8.19 (d, J=8.1 Hz, 1H), 7.67 (ddd, J=1.3, 4.3, 8.6 Hz, 1H), 7.31 (d, J=8.1 Hz, 1H), 7.15 (s, 1H), 7.09 (d, J=2.6 Hz, 1H), 5.54-5.33 (m, 1H), 4.72 (s, 2H), 4.60-4.51 (m, 1H), 4.43-4.33 (m, 1H), 4.28-4.15 (m, 2H), 3.80-3.61 (m, 2H), 3.58-3.42 (m, 4H), 3.41-3.35 (m, 1H), 2.90-2.71 (m, 2H), 2.58 (s, 3H), 2.44-2.30 (m, 1H), 2.16-1.95 (m, 1H), 1.36-1.29 (m, 3H).

### Example 34

### trans-5-[2S,6R)-2-[[[4-fluoro-1-[2-(hydroxymethyl)-6-methyl-4-pyridyl]-3-piperidyl]amino]methyl]-6-methyl-morpholin-4-yl]quinoline-8-carbonitrile

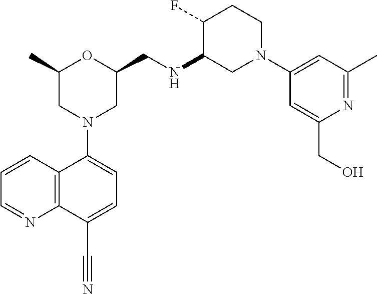 [SMILES: Cc1cc(N2CC[C@@H](F)[C@H](NC[C@H]3CN(c4ccc(C#N)c5ncccc45)C[C@@H](C)O3)C2)cc(CO)n1]

The title compound was prepared in analogy to the preparation of Example 26 by using trans-tert-butyl N-(4-fluoro-3-piperidyl)carbamate (CAS: 1052713-46-8, Vendor: PharmaBlock) instead of trans-tert-butyl (3-hydroxypiperidin-4-yl)carbamate (compound 26a). Example 34 (5 mg) was obtained. MS: calc'd 505 (MH₊), measured 505 (MH₊). ¹H NMR (400 MHz, METHANOL-d₄) δ=9.03-8.99 (m, 1H), 8.67 (ddd, J=1.7, 3.3, 8.6 Hz, 1H), 8.21-8.16 (m, 1H), 7.69-7.63 (m, 1H), 7.32-7.27 (m, 1H), 7.15 (dd, J=2.5, 6.3 Hz, 1H), 7.09 (dd, J=2.6, 10.1 Hz, 1H), 5.26-5.02 (m, 1H), 4.73 (s, 2H), 4.70-4.59 (m, 1H), 4.43-4.27 (m, 2H), 4.19 (ddd, J=2.0, 6.2, 10.0 Hz, 1H), 3.68 (dt, J=3.7, 9.8 Hz, 1H), 3.56-3.34 (m, 6H), 2.91-2.70 (m, 2H), 2.58 (s, 3H), 2.51-2.41 (m, 1H), 2.02-1.90 (m, 1H), 1.36-1.26 (m, 3H).

### Example 35

### 5-[2S,6R)-2-[[[(3R)-1-[2-(hydroxymethyl)-6-methyl-4-pyridyl]-3-piperidyl]-methyl-amino]methyl]-6-methyl-morpholin-4-yl]quinoline-8-carbonitrile

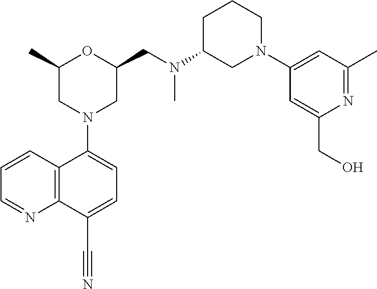 [SMILES: Cc1cc(N2CCC[C@@H](N(C)C[C@H]3CN(c4ccc(C#N)c5ncccc45)C[C@@H](C)O3)C2)cc(CO)n1]

The title compound was prepared in analogy to the preparation of Example 26 by using tert-butyl N-methyl-N-[(3R)-3-piperidyl]carbamate (CAS: 309962-67-2, Vendor: PharmaBlock) instead of trans-tert-butyl (3-hydroxypiperidin-4-yl)carbamate (compound 26a). Example 35 (5 mg) was obtained. MS: calc'd 501 (MH₊), measured 501 (MH₊). ¹H NMR (400 MHz, METHANOL-d₄) δ=9.00 (dd, J=1.5, 4.2 Hz, 1H), 8.67 (dd, J=1.3, 8.6 Hz, 1H), 8.17 (d, J=8.1 Hz, 1H), 7.65 (dd, J=4.3, 8.6 Hz, 1H), 7.28 (d, J=8.1 Hz, 1H), 7.14-7.08 (m, 1H), 7.07-7.02 (m, 1H), 4.72 (s, 2H), 4.63 (br d, J=12.6 Hz, 1H), 4.56-4.46 (m, 1H), 4.30 (br d, J=13.6 Hz, 1H), 4.24-4.15 (m, 1H), 3.69-3.39 (m, 6H), 3.30-3.15 (m, 1H), 3.11 (s, 3H), 2.85-2.70 (m, 2H), 2.62-2.52 (m, 3H), 2.42-2.31 (m, 1H), 2.11 (br d, J=14.1 Hz, 1H), 2.06-1.93 (m, 1H), 1.85-1.69 (m, 1H), 1.30 (d, J=6.2 Hz, 3H).

### Example 36

### 5-[2S,6R)-2-[[[5,5-difluoro-1-[2-(hydroxymethyl)-6-methyl-4-pyridyl]-3-piperidyl]amino]methyl]-6-methyl-morpholin-4-yl]quinoline-8-carbonitrile

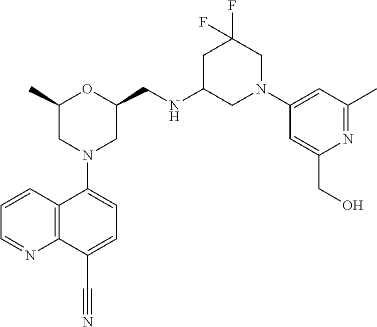 [SMILES: Cc1cc(N2CC(NC[C@H]3CN(c4ccc(C#N)c5ncccc45)C[C@@H](C)O3)CC(F)(F)C2)cc(CO)n1]

The title compound was prepared in analogy to the preparation of Example 26 by using tert-butyl N-(5,5-difluoro-3-piperidyl)carbamate (CAS: 1303973-94-5, Vendor: PharmaBlock) instead of trans-tert-butyl (3-hydroxypiperidin-4-yl)carbamate (compound 26a). Example 36 (9 mg) was obtained. MS: calc'd 523 (MH₊), measured 523 (MH₊). ¹H NMR (400 MHz, METHANOL-d₄) δ=9.02 (dd, J=1.7, 4.2 Hz, 1H), 8.67 (dd, J=1.6, 8.6 Hz, 1H), 8.19 (d, J=8.1 Hz, 1H), 7.67 (dd, J=4.2, 8.6 Hz, 1H), 7.30 (d, J=8.1 Hz, 1H), 7.25 (t, J=3.0 Hz, 1H), 7.20 (d, J=2.8 Hz, 1H), 4.75 (s, 2H), 4.66-4.55 (m, 2H), 4.42-4.31 (m, 1H), 4.22-4.13 (m, 1H), 3.85-3.66 (m, 2H), 3.66-3.55 (m, 1H), 3.51-3.39 (m, 3H), 3.38-3.34 (m, 1H), 2.94-2.72 (m, 3H), 2.61 (s, 3H), 2.52-2.32 (m, 1H), 1.33 (d, J=6.2 Hz, 3H).

### Example 37

### 5-[2S,6R)-2-[[[1-[2-(hydroxymethyl)-6-methyl-4-pyridyl]azetidin-3-yl]-methyl-amino]methyl]-6-methyl-morpholin-4-yl]quinoline-8-carbonitrile

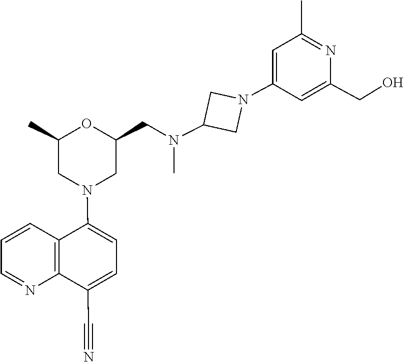 [SMILES: Cc1cc(N2CC(N(C)C[C@H]3CN(c4ccc(C#N)c5ncccc45)C[C@@H](C)O3)C2)cc(CO)n1]

The title compound was prepared in analogy to the preparation of Example 26 by using tert-butyl N-(azetidin-3-yl)-N-methyl-carbamate (CAS: 577777-20-9, Vendor: PharmaBlock) instead of trans-tert-butyl (3-hydroxypiperidin-4-yl)carbamate (compound 26a). Example 37 (31 mg) was obtained. MS: calc'd 473 (MH₊), measured 473 (MH₊). ¹H NMR (400 MHz, METHANOL-d₄) δ=9.00 (dd, J=1.6, 4.3 Hz, 1H), 8.69 (dd, J=1.7, 8.6 Hz, 1H), 8.17 (d, J=7.9 Hz, 1H), 7.66 (dd, J=4.3, 8.6 Hz, 1H), 7.28 (d, J=7.9 Hz, 1H), 6.63 (d, J=2.2 Hz, 1H), 6.53 (d, J=2.1 Hz, 1H), 4.69 (s, 2H), 4.66-4.46 (m, 6H), 4.25-4.16 (m, 1H), 3.51-3.40 (m, 2H), 3.39-3.35 (m, 2H), 3.07 (s, 3H), 2.84-2.70 (m, 2H), 2.55 (s, 3H), 1.32 (d, J=6.2 Hz, 3H).

### Example 38

### 5-[2S,6R)-2-[[[(3S)-1-[2-(hydroxymethyl)-6-methyl-4-pyridyl]-3-piperidyl]-methyl-amino]methyl]-6-methyl-morpholin-4-yl]quinoline-8-carbonitrile

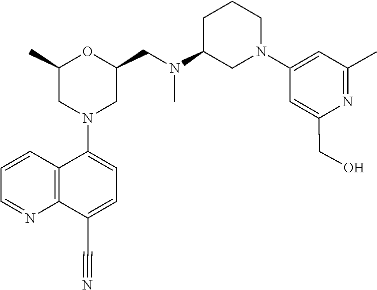 [SMILES: Cc1cc(N2CCC[C@H](N(C)C[C@H]3CN(c4ccc(C#N)c5ncccc45)C[C@@H](C)O3)C2)cc(CO)n1]

The title compound was prepared in analogy to the preparation of Example 26 by using tert-butyl N-methyl-N-[(3S)-3-piperidyl]carbamate (CAS: 309962-63-8, Vendor: PharmaBlock) instead of trans-tert-butyl (3-hydroxypiperidin-4-yl)carbamate (compound 26a). Example 38 (13 mg) was obtained. MS: calc'd 501 (MH₊), measured 501 (MH₊). ¹H NMR (400 MHz, METHANOL-d₄) δ=8.99 (dd, J=1.6, 4.2 Hz, 1H), 8.67 (dd, J=1.6, 8.6 Hz, 1H), 8.16 (d, J=7.9 Hz, 1H), 7.65 (dd, J=4.3, 8.6 Hz, 1H), 7.28 (d, J=8.1 Hz, 1H), 7.10 (d, J=2.6 Hz, 1H), 7.04 (d, J=2.6 Hz, 1H), 4.74-4.70 (m, 1H), 4.71 (s, 1H), 4.70-4.62 (m, 1H), 4.51 (br t, J=10.0 Hz, 1H), 4.33-4.14 (m, 2H), 3.69-3.38 (m, 6H), 3.29-3.15 (m, 1H), 3.10 (s, 3H), 2.84-2.70 (m, 2H), 2.63-2.50 (m, 3H), 2.40-2.27 (m, 1H), 2.16-1.95 (m, 2H), 1.82-1.68 (m, 1H), 1.31 (d, J=6.4 Hz, 3H).

### Example 39

### 4-[2S,6R)-2-[[2-benzyl-4-(2,6-dimethyl-4-pyridyl)piperazin-1-yl]methyl]-6-methyl-morpholin-4-yl]pyrazolo[1,5-a]pyridine-7-carbonitrile

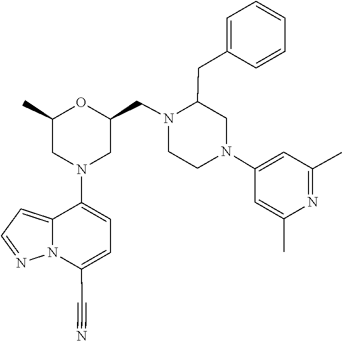 [SMILES: Cc1cc(N2CCN(C[C@H]3CN(c4ccc(C#N)n5nccc45)C[C@@H](C)O3)C(Cc3ccccc3)C2)cc(C)n1]

The title compound was prepared according to the following scheme:

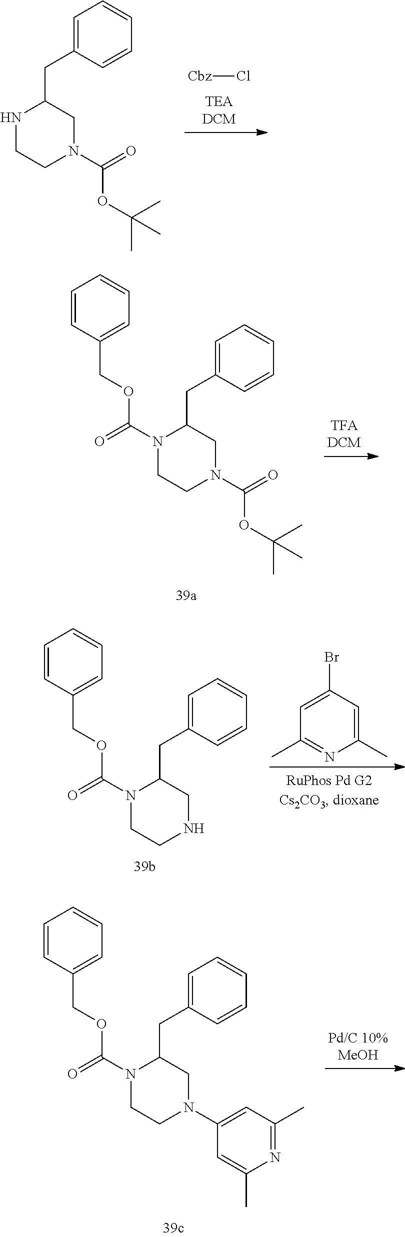

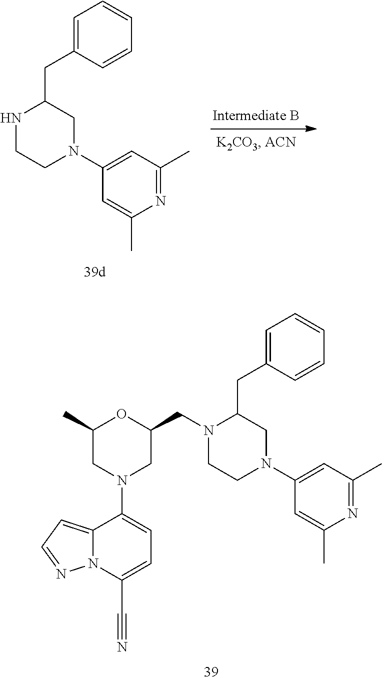 [SMILES: Cc1cc(N2CCNC(Cc3ccccc3)C2)cc(C)n1 ; Cc1cc(N2CCN(C[C@H]3CN(c4ccc(C#N)n5nccc45)C[C@@H](C)O3)C(Cc3ccccc3)C2)cc(C)n1]

Step 1: preparation of benzyl 2-benzylpiperazine-1-carboxylate (compound 39b)

To a flask was added tert-butyl 3-benzylpiperazine-1-carboxylate (CAS: 502649-29-8, Vendor: BePharm, 300 mg, 1.09 mmol), TEA (330 mg, 454 μL, 3.26 mmol) and DCM (2 mL). Then it was cooled with ice bath and Cbz-Cl (278 mg, 232 μL, 1.63 mmol) was added drop-wise. After being warmed to rt slowly and stirred for 2 hrs, the reaction mixture was diluted with 20 mL water and extracted with EA (15 mL) twice, the organic layer was dried over Na₂SO₄ and concentrated to give a brown oil. After purification by flash column (EA/PE, 0 to 20%), the compound 39a (268 mg) was obtained as an oil. MS: calc'd 411 (MH₊), measured 411 (MH₊).

The compound 39a was dissolved in DCM (2 mL) and TFA (124 mg, 84 μL, 1.09 mmol). The mixture was stirred at rt for 2 hrs, and then concentrated directly to give compound 39b (200 mg) as an oil. MS: calc'd 311 (MH₊), measured 311 (MH₊).

Step 2: preparation of 3-benzyl-1-(2,6-dimethyl-4-pyridyl)piperazine (compound 39d)

To a flask was added benzyl 2-benzylpiperazine-1-carboxylate (compound 39b, 65 mg, 209 μmol), 4-bromo-2,6-dimethylpyridine (39 mg, 209 μmol), Cs₂CO₃ (205 mg, 628 μmol) and 1,4-dioxane (5 mL), the suspension was bubbled with N₂ for 5 mins and Ruphos Pd G2 (16 mg, 21 μmol) was added. The mixture was heated to 90° C. and stirred for 12 hrs. After being cooled down, the mixture was diluted with 10 mL EA and filtered through celite, and the filtrate was concentrated to give a yellow oil, which was purified by flash column (EA/PE=0 to 100% & MeOH/EA=10%), the elution was concentrated to give compound 39c (52 mg) as an oil. MS: calc'd 416 (MH₊), measured 416 (MH₊).

To a flask containing benzyl 2-benzyl-4-(2,6-dimethyl-4-pyridyl)piperazine-1-carboxylate (compound 39c, 26 mg, 63 μmol) was added Pd/C (10 wt. %, 10 mg, 71 μmol) and MeOH (5 mL). The reaction mixture was stirred at r.t. under hydrogen balloon for 2 hrs, then filtered through celite, and the filtrate was concentrated to give compound 39d (17 mg) as a semi-solid. MS: calc'd 282 (MH₊), measured 282 (MH₊).

Step 3: preparation of 4-[(2S,6R)-2-[[2-benzyl-4-(2,6-dimethyl-4-pyridyl)piperazin-1-yl]methyl]-6-methyl-morpholin-4-yl]pyrazolo[1,5-a]pyridine-7-carbonitrile (Example 39)

To a tube was added [2R,6R)-4-(7-cyanopyrazolo[1,5-a]pyridin-4-yl)-6-methyl-morpholin-2-yl]methyl trifluoromethanesulfonate (Intermediate B, 20 mg, 50 μmol), potassium carbonate (27 mg, 198 μmol), 3-benzyl-1-(2,6-dimethyl-4-pyridyl)piperazine (compound 39d, 21 mg, 74 μmol) and ACN (4 mL). The suspension was heated to reflux for 2 hrs. After being cooled down, the mixture was diluted with some ACN and filtered through celite, the filtrate was concentrated to give a yellow solid which was purified by prep-HPLC to give Example 39 (2 mg) as a light yellow powder. MS: calc'd 536 (MH₊), measured 536 (MH₊). ¹H NMR (400 MHz, METHANOL-d₄) δ=8.61-8.47 (m, 1H), 8.03 (s, 1H), 7.51-7.45 (m, 1H), 7.33-7.15 (m, 5H), 6.84 (t, J=2.8 Hz, 1H), 6.56 (dd, J=2.3, 7.9 Hz, 2H), 3.98-3.88 (m, 1H), 3.81-3.69 (m, 2H), 3.53-3.39 (m, 1H), 3.26-3.20 (m, 2H), 3.19-3.07 (m, 3H), 3.06-2.86 (m, 3H), 2.86-2.72 (m, 2H), 2.72-2.58 (m, 3H), 2.45-2.36 (m, 6H), 1.28 (dd, J=3.6, 6.2 Hz, 3H).

### Example 40

### 5-[(2S,6R)-2-[[4-(2-amino-6-methyl-4-pyridyl)piperazin-1-yl]methyl]-6-methyl-morpholin-4-yl]quinoline-8-carbonitrile

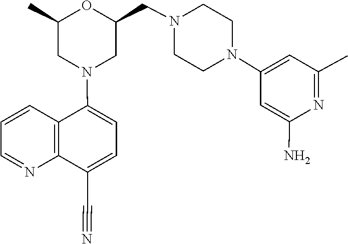 [SMILES: Cc1cc(N2CCN(C[C@H]3CN(c4ccc(C#N)c5ncccc45)C[C@@H](C)O3)CC2)cc(N)n1]

The title compound was prepared in analogy to the preparation of Example 19 by using 4-chloro-6-methylpyridin-2-amine (CAS: 5600-21-5, Vendor: Accela ChemBio Inc) instead of 4-chloropyridin-2-amine (compound 19a). Example 40 (39 mg) was obtained as a light yellow powder. MS: calc'd 458 (MH₊), measured 458 (MH₊). ¹H NMR (400 MHz, METHANOL-d₄) δ=9.01 (dd, J=1.7, 4.2 Hz, 1H), 8.67 (dd, J=1.6, 8.6 Hz, 1H), 8.18 (d, J=7.9 Hz, 1H), 7.67 (dd, J=4.3, 8.6 Hz, 1H), 7.30 (d, J=8.1 Hz, 1H), 6.51 (d, J=1.7 Hz, 1H), 6.02 (d, J=2.2 Hz, 1H), 4.55-4.45 (m, 1H), 4.25-4.16 (m, 1H), 3.87 (br s, 4H), 3.74-3.48 (m, 4H), 3.47-3.38 (m, 4H), 2.84-2.70 (m, 2H), 2.40 (s, 3H), 1.33 (d, J=6.2 Hz, 3H).

### Example 41

### 5-[2S,6R)-2-[[(2R)-4-(2-amino-6-methyl-4-pyridyl)-2-(hydroxymethyl)piperazin-1-yl]methyl]-6-methyl-morpholin-4-yl]quinoline-8-carbonitrile

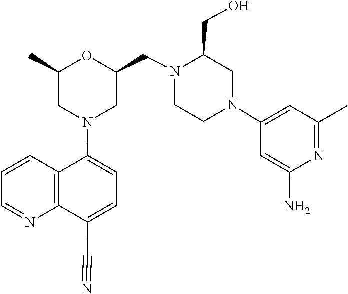 [SMILES: Cc1cc(N2CCN(C[C@H]3CN(c4ccc(C#N)c5ncccc45)C[C@@H](C)O3)[C@@H](CO)C2)cc(N)n1]

The title compound was prepared in analogy to the preparation of Example 19 by using 4-chloro-6-methylpyridin-2-amine and tert-butyl (2R)-2-(hydroxymethyl)piperazine-1-carboxylate (CAS: 169448-87-7, Vendor: BePharm) instead of 4-chloropyridin-2-amine (compound 19a) and tert-butyl piperazine-1-carboxylate. Example 41 (12 mg) was obtained as a light yellow powder. MS: calc'd 488 (MH₊), measured 488 (MH₊). ¹H NMR (400 MHz, METHANOL-d₄) δ=8.99 (dd, J=1.5, 4.2 Hz, 1H), 8.68 (dd, J=1.6, 8.6 Hz, 1H), 8.17 (d, J=7.9 Hz, 1H), 7.65 (dd, J=4.2, 8.6 Hz, 1H), 7.27 (d, J=8.2 Hz, 1H), 6.18 (s, 1H), 5.85 (d, J=2.2 Hz, 1H), 4.23-4.14 (m, 1H), 4.14-4.07 (m, 1H), 3.75-3.67 (m, 2H), 3.64-3.58 (m, 1H), 3.54-3.44 (m, 3H), 3.44-3.37 (m, 2H), 3.19-3.10 (m, 2H), 3.00 (dd, J=7.0, 14.0 Hz, 1H), 2.77-2.67 (m, 3H), 2.65-2.57 (m, 1H), 2.25 (s, 3H), 1.28 (d, J=6.2 Hz, 3H).

### Example 42

### 5-[(2R,6S)-2-methyl-6-[[4-[2-methyl-6-(methylamino)-4-pyridyl]piperazin-1-yl]methyl]morpholin-4-yl]quinoline-8-carbonitrile

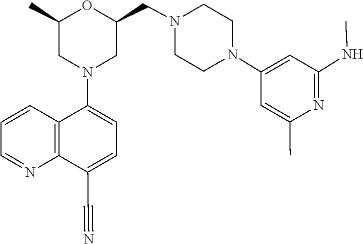 [SMILES: CNc1cc(N2CCN(C[C@H]3CN(c4ccc(C#N)c5ncccc45)C[C@@H](C)O3)CC2)cc(C)n1]

The title compound was prepared according to the following scheme:

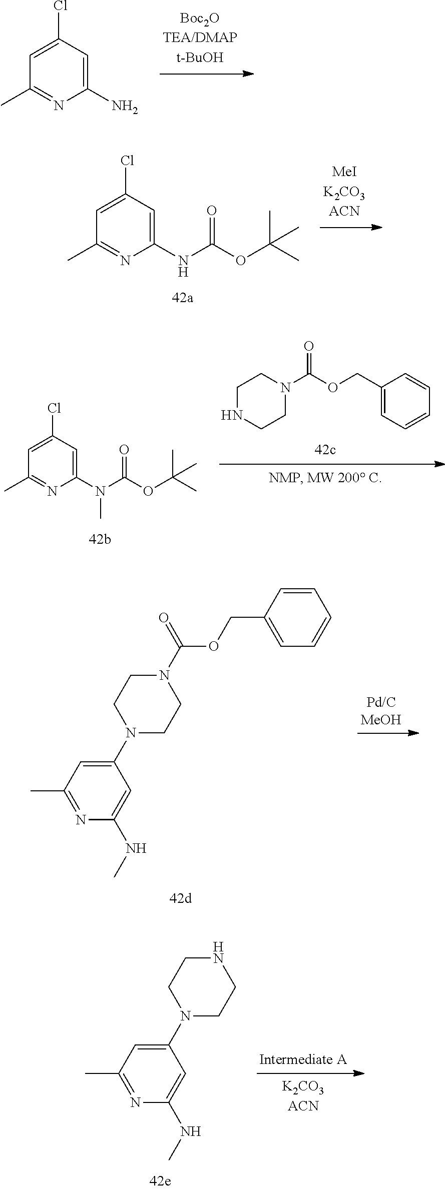

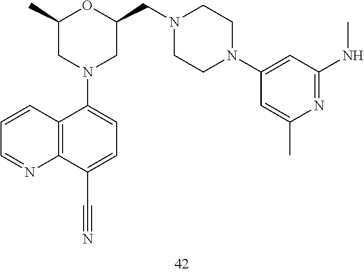 [SMILES: CNc1cc(N2CCN(C[C@H]3CN(c4ccc(C#N)c5ncccc45)C[C@@H](C)O3)CC2)cc(C)n1]

Step 1: preparation of tert-butyl N-(4-chloro-6-methyl-2-pyridyl)carbamate (compound 42a)

To a flask was added 4-chloro-6-methylpyridin-2-amine (200 mg, 1.40 mmol), t-BuOH (5 mL), DMAP (17 mg, 140 μmol), TEA (284 mg, 391 μL, 2.81 mmol) and Boc₂O (459 mg, 2.10 mmol). After being stirred at rt for 12 hrs, the mixture was concentrated to give an oil and purified by flash column (EA/PE=0 to 20%) to give compound 42a (307 mg) as a colorless oil. MS: calc'd 243 (MH₊), measured 243 (MH₊).

Step 2: preparation of tert-butyl N-(4-chloro-6-methyl-2-pyridyl)-N-methyl-carbamate (compound 42b)

To a flask was added tert-butyl N-(4-chloro-6-methyl-2-pyridyl)carbamate (compound 42a, 100 mg, 412 μmol) and THF (4 mL), the solution was cooled with ice bath and NaH on oil (60%, 82.4 mg, 2.06 mmol) was added. After being stirred for 10 mins, the reaction mixture was added with iodomethane (234 mg, 1.65 mmol), then warmed to rt slowly and stirred for another 12 hrs. The reaction mixture was quenched with water and extracted with EA (15 mL) three times, the organic layer was dried over Na₂SO₄ and concentrated to give a yellow oil, which was purified by flash column (EA/PE=0 to 5%) to give compound 42b (87 mg) as an oil. MS: calc'd 257 (MH₊), measured 257 (MH₊).

Step 3: preparation of benzyl 4-[2-methyl-6-(methylamino)-4-pyridyl]piperazine-1-carboxylate (compound 42d)

To a microwave tube was added tert-butyl N-(4-chloro-6-methyl-2-pyridyl)-N-methyl-carbamate (compound 42b, 87 mg, 339 μmol), benzyl piperazine-1-carboxylate (compound 42c, 97 mg, 441 μmol) and NMP (5 mL), the tube was sealed and stirred under microwave at 200° C. for 30 mins. After being cooled down, the mixture was poured into 20 mL water and adjusted to pH >7, then it was extracted with DCM (15 mL) twice, the organic layer was dried and concentrated to give a brown crude product which was purified by flash column (EA/PE=0 to 100% & MeOH/DCM=20%) to give compound 42d as a brown oil (80 mg). MS: calc'd 341 (MH₊), measured 341 (MH₊).

Step 4: preparation of N,6-dimethyl-4-piperazin-1-yl-pyridin-2-amine (compound 42e)

To a flask was added benzyl 4-[2-methyl-6-(methylamino)-4-pyridyl]piperazine-1-carboxylate (compound 42d, 80 mg, 235 μmol), Pd/C (10 wt. %, 10 mg, 71 μmol) and MeOH (2 mL), the solution was purged with H₂ for 3 times and stirred at rt for 2 hrs. Then the mixture was filtered and concentrated to give compound 42e (50 mg) as an oil. MS: calc'd 207 (MH₊), measured 207 (MH₊).

Step 5: preparation of 5-[(2R,6S)-2-methyl-6-[[4-[2-methyl-6-(methylamino)-4-pyridyl]piperazin-1-yl]methyl]morpholin-4-yl]quinoline-8-carbonitrile (Example 42)

To a flask was added [(2R,6R)-4-(8-cyano-5-quinolyl)-6-methyl-morpholin-2-yl]methyl trifluoromethanesulfonate (Intermediate A, 50 mg, 120 μmol), N,6-dimethyl-4-piperazin-1-yl-pyridin-2-amine (compound 42e, 25 mg, 120 μmol), potassium carbonate (50 mg, 361 μmol) and ACN (4 mL), the mixture was stirred at 85° C. for 2 hrs. Then the mixture was cooled down and filtered through celite, the filtrate was concentrated to give a yellow oil which was purified by prep-HPLC to give Example 42 (31 mg) as a light yellow powder. MS: calc'd 472 (MH₊), measured 472 (MH₊). ¹H NMR (400 MHz, METHANOL-d₄) δ=8.99 (dd, J=1.7, 4.2 Hz, 1H), 8.66 (dd, J=1.7, 8.6 Hz, 1H), 8.16 (d, J=7.9 Hz, 1H), 7.65 (dd, J=4.3, 8.6 Hz, 1H), 7.28 (d, J=7.9 Hz, 1H), 6.48 (d, J=1.5 Hz, 1H), 5.93 (d, J=2.2 Hz, 1H), 4.50 (dt, J=5.3, 7.7 Hz, 1H), 4.24-4.14 (m, 1H), 4.06-3.71 (m, 4H), 3.65-3.46 (m, 4H), 3.45-3.36 (m, 4H), 2.96 (s, 3H), 2.83-2.69 (m, 2H), 2.40 (s, 3H), 1.32 (d, J=6.2 Hz, 3H).

### Example 43

### 5-[(2S,6R)-2-[[4-(2-amino-6-methyl-4-pyridyl)-1-piperidyl]methyl]-6-methyl-morpholin-4-yl]quinoline-8-carbonitrile

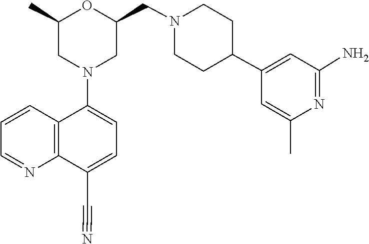 [SMILES: Cc1cc(C2CCN(C[C@H]3CN(c4ccc(C#N)c5ncccc45)C[C@@H](C)O3)CC2)cc(N)n1]

The title compound was prepared according to the following scheme:

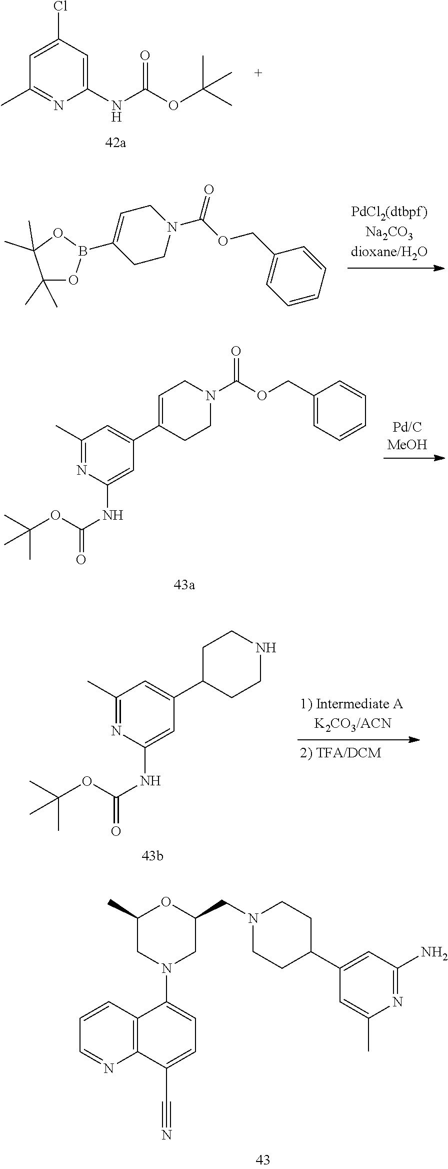 [SMILES: Cc1cc(Cl)cc(NC(=O)OC(C)(C)C)n1 ; CC1(C)OB(C2=CCN(C(=O)OCc3ccccc3)CC2)OC1(C)C ; Cc1cc(C2=CCN(C(=O)OCc3ccccc3)CC2)cc(NC(=O)OC(C)(C)C)n1 ; Cc1cc(C2CCNCC2)cc(NC(=O)OC(C)(C)C)n1 ; Cc1cc(C2CCN(C[C@H]3CN(c4ccc(C#N)c5ncccc45)C[C@@H](C)O3)CC2)cc(N)n1]

Step 1: preparation of benzyl 4-[2-(tert-butoxycarbonylamino)-6-methyl-4-pyridyl]-3,6-dihydro-2H-pyridine-1-carboxylate (compound 43a) To a tube was added tert-butyl N-(4-chloro-6-methyl-2-pyridyl)-N-methyl-carbamate (compound 42a, 85 mg, 350 μmol), benzyl 4-(4,4,5,5-tetramethyl-1,3,2-dioxaborolan-2-yl)-3,6-dihydropyridine-1(2H)-carboxylate (CAS: 286961-15-7, Vendor: Bidepharm, 180 mg, 525 μmol), sodium carbonate (111 mg, 1.05 mmol), 1,4-dioxane (4 mL) and water (0.40 mL), the suspension was bubbled with N₂ for 5 mins and 1,1′-bis(di-tert-butylphosphino)ferrocene palladium dichloride (23 mg, 35 μmol) was added. After being stirred at 90° C. for 16 hrs, the mixture was cooled down and filtered, the filtrate was concentrated to give an oil which was purified by flash column (EA/PE=0 to 35%) to give compound 43a (112 mg) as an oil. MS: calc'd 424 (MH₊), measured 424 (MH₊).

Step 2: preparation of tert-butyl N-[6-methyl-4-(4-piperidyl)-2-pyridyl]carbamate (compound 43b)

The compound 43a was dissolved in MeOH (4 mL) and Pd/C (10 wt. %, 15 mg, 107 μmol) was added. The mixture was sucking in vacuum and purged with H₂ for 3 times, then it was stirred at rt for 2 hrs. The mixture was filtered and the filtrate was concentrated to give compound 43b (76 mg) as an oil. MS: calc'd 292 (MH₊), measured 292 (MH₊).

Step 3: preparation of 5-[(2S,6R)-2-[[4-(2-amino-6-methyl-4-pyridyl)-1-piperidyl]methyl]-6-methyl-morpholin-4-yl]quinoline-8-carbonitrile (Example 43)

To a flask was added [(2R,6R)-4-(8-cyano-5-quinolyl)-6-methyl-morpholin-2-yl]methyl trifluoromethanesulfonate (Intermediate A, 50 mg, 120 μmol), tert-butyl N-[6-methyl-4-(4-piperidyl)-2-pyridyl]carbamate (compound 43b, 42 mg, 144 μmol), potassium carbonate (50 mg, 361 μmol) and ACN (4 mL), the mixture was stirred at 85° C. for 2 hrs. Then the mixture was cooled down and filtered through celite, the filtrate was concentrated to give a yellow oil. The oil was dissolved in DCM (2 mL) and TFA (738 mg, 500 μL, 6.47 mmol), then it was stirred at rt for 2 hrs. The mixture was concentrated directly to give an oil which was purified via prep-HPLC to give Example 43 (19 mg) as a light yellow powder. MS: calc'd 457 (MH₊), measured 457 (MH₊). ¹H NMR (400 MHz, METHANOL-d₄) δ=9.02 (dd, J=1.7, 4.2 Hz, 1H), 8.69 (dd, J=1.6, 8.6 Hz, 1H), 8.19 (d, J=7.9 Hz, 1H), 7.67 (dd, J=4.2, 8.6 Hz, 1H), 7.31 (d, J=8.1 Hz, 1H), 6.80-6.69 (m, 2H), 4.58-4.47 (m, 1H), 4.26-4.15 (m, 1H), 3.95-3.81 (m, 2H), 3.45 (br d, J=11.4 Hz, 2H), 3.38 (br d, J=6.2 Hz, 2H), 3.31-3.18 (m, 2H), 3.05-2.94 (m, 1H), 2.85-2.70 (m, 2H), 2.50 (s, 3H), 2.27-1.97 (m, 4H), 1.34 (d, J=6.2 Hz, 3H).

### Example 44

### 5-[(2S,6R)-2-[[2-[2-(1-hydroxyethyl)-6-methyl-4-pyridyl]-2,6-diazaspiro[3.3]heptan-6-yl]methyl]-6-methyl-morpholin-4-yl]quinoline-8-carbonitrile

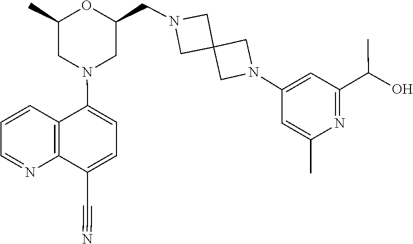 [SMILES: Cc1cc(N2CC3(CN(C[C@H]4CN(c5ccc(C#N)c6ncccc56)C[C@@H](C)O4)C3)C2)cc(C(C)O)n1]

The title compound was prepared in analogy to the preparation of Example 1 by using 1-(4-bromo-6-methyl-2-pyridyl)ethanol (compound 44b) instead of (4-bromo-6-methyl-2-pyridyl)methanol (compound 1a). Example 44 (17 mg) was obtained as a light yellow powder. MS: calc'd 499 (MH₊), measured 499 (MH₊). ¹H NMR (400 MHz, METHANOL-d₄) δ=9.00 (dd, J=1.5, 4.2 Hz, 1H), 8.65 (dd, J=1.5, 8.6 Hz, 1H), 8.16 (d, J=7.9 Hz, 1H), 7.66 (dd, J=4.2, 8.6 Hz, 1H), 7.27 (d, J=7.9 Hz, 1H), 6.49 (d, J=2.1 Hz, 1H), 6.40 (d, J=1.7 Hz, 1H), 4.45-4.37 (m, 4H), 4.23-4.02 (m, 6H), 3.43-3.39 (m, 1H), 3.37 (s, 4H), 2.76 (br t, J=11.0 Hz, 1H), 2.68 (br t, J=11.1 Hz, 1H), 2.51 (s, 3H), 1.51 (d, J=6.6 Hz, 3H), 1.29 (d, J=6.1 Hz, 3H).

The compound 44b was prepared according to the following scheme:

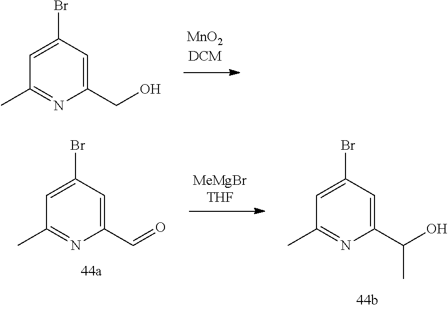

Step 1: preparation of 4-bromo-6-methyl-pyridine-2-carbaldehyde (compound 44a)

To a flask was added (4-bromo-6-methyl-2-pyridyl)methanol (500 mg, 2.47 mmol), manganese dioxide (1.08 g, 12.40 mmol) and DCM (10 mL), a dark suspension was formed. The mixture was heated to reflux and stirred for 10 hrs. Then it was cooled and filtered, the filtrate was concentrated to give compound 44a (372 mg) as a white solid. MS: calc'd 200 (MH₊), measured 200 (MH₊).

Step 2: preparation of 1-(4-bromo-6-methyl-2-pyridyl)ethanol (compound 44b)

To a flask was added 4-bromo-6-methyl-pyridine-2-carbaldehyde (compound 44a, 100 mg, 500 μmol) and THF (3 mL), a pale yellow solution was formed, then it was cooled with dry ice/ethanol bath and methylmagnesium bromide (1 M in THF, 1 mL, 1.00 mmol) was added portion wise. The mixture was warmed to rt slowly and stirred for 2 hrs. Then it was quenched with sat. NH₄Cl and diluted with 20 mL water. The mixture was extracted with EA (20 mL) twice, the organic layer was dried over Na₂SO₄ and concentrated to give compound 44b (105 mg) as a crude oil. MS: calc'd 216 (MH₊), measured 216 (MH₊).

### Example 45

### 5-[(2S,6R)-2-[[4-[2-amino-6-(hydroxymethyl)-4-pyridyl]piperazin-1-yl]methyl]-6-methyl-morpholin-4-yl]quinoline-8-carbonitrile

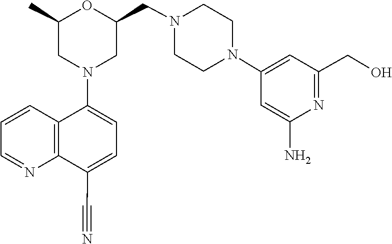 [SMILES: C[C@@H]1CN(c2ccc(C#N)c3ncccc23)C[C@H](CN2CCN(c3cc(N)nc(CO)c3)CC2)O1]

The title compound was prepared according to the following scheme:

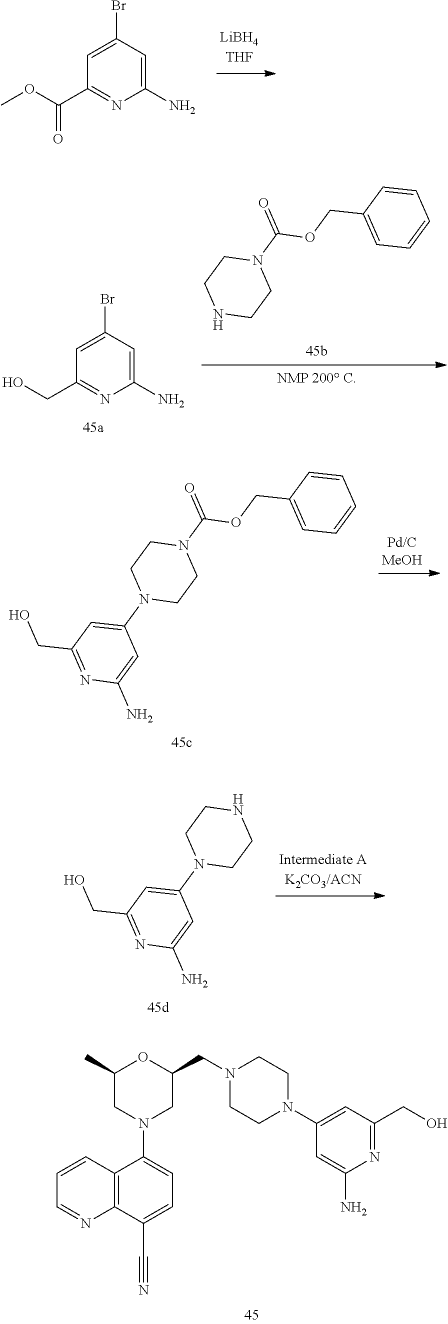 [SMILES: COC(=O)c1cc(Br)cc(N)n1 ; Nc1cc(Br)cc(CO)n1 ; O=C(OCc1ccccc1)N1CCNCC1 ; Nc1cc(N2CCN(C(=O)OCc3ccccc3)CC2)cc(CO)n1 ; Nc1cc(N2CCNCC2)cc(CO)n1 ; C[C@@H]1CN(c2ccc(C#N)c3ncccc23)C[C@H](CN2CCN(c3cc(N)nc(CO)c3)CC2)O1]

Step 1: preparation of (6-amino-4-bromo-2-pyridyl)methanol (compound 45a)

To a microwave tube was added methyl 6-amino-4-bromopicolinate (CAS: 885326-88-5, Vendor: PharmBlock, 231 mg, 1.00 mmol), THF (3 mL) and LiBH₄ (2 M in THF, 1 mL, 2.00 mmol). After being stirred at 65° C. for 2 hrs under microwave, the reaction was quenched by adding Na₂SO₄.10H₂O and the mixture was stirred at rt for another hour. Then it was filtered through celite and the filtrate was concentrated to give compound 45a (230 mg) as a crude oil. MS: calc'd 203 (MH₊), measured 203 (MH₊).

Step 2: preparation of benzyl 4-[2-amino-6-(hydroxymethyl)-4-pyridyl]piperazine-1-carboxylate (compound 45c)

To a microwave tube was added (6-amino-4-bromo-2-pyridyl)methanol (compound 45a, 80 mg, 394 μmol), benzyl piperazine-1-carboxylate (compound 45b, 87 mg, 394 μmol) and NMP (4 mL), the tube was sealed and stirred under microwave at 200° C. for 1 h. After being cooled down, the mixture was purified via prep-HPLC to give compound 45c (25 mg) as a pale yellow powder. MS: calc'd 343 (MH₊), measured 343 (MH₊).

Step 3: preparation of (6-amino-4-piperazin-1-yl-2-pyridyl)methanol (compound 45d)

The compound 45c was dissolved in MeOH (4 mL) and Pd/C (10 wt. %, 10 mg, 71 μmol) was added. The mixture was purged with H₂ for 3 times, and then stirred at rt for 2 hrs. The mixture was filtered and the filtrate was concentrated to give compound 45d (15 mg) as an oil. MS: calc'd 209 (MH₊), measured 209 (MH₊).

Step 4: preparation of 5-[(2S,6R)-2-[[4-[2-amino-6-(hydroxymethyl)-4-pyridyl]piperazin-1-yl]methyl]-6-methyl-morpholin-4-yl]quinoline-8-carbonitrile (Example 45)

To a flask was added [(2R,6R)-4-(8-cyano-5-quinolyl)-6-methyl-morpholin-2-yl]methyl trifluoromethanesulfonate (Intermediate A, 28 mg, 67 μmol), (6-amino-4-piperazin-1-yl-2-pyridyl)methanol (compound 45d, 14 mg, 67 μmol), potassium carbonate (19 mg, 135 μmol) and ACN (4 mL), the mixture was stirred at 85° C. for 2 hrs. After being cooled down, the mixture was filtered, and the filtrate was concentrated to give a yellow oil which was purified by prep-HPLC to give Example 45 (20 mg) as a light yellow powder. MS: calc'd 474 (MH₊), measured 474 (MH₊). ¹H NMR (400 MHz, METHANOL-d₄) δ=9.00 (dd, J=1.6, 4.3 Hz, 1H), 8.66 (dd, J=1.7, 8.6 Hz, 1H), 8.17 (d, J=7.9 Hz, 1H), 7.65 (dd, J=4.3, 8.6 Hz, 1H), 7.28 (d, J=8.1 Hz, 1H), 6.58 (d, J=2.3 Hz, 1H), 6.07 (d, J=2.4 Hz, 1H), 4.60 (s, 2H), 4.55-4.47 (m, 1H), 4.24-4.14 (m, 1H), 3.89 (br s, 4H), 3.59 (br s, 4H), 3.46-3.39 (m, 4H), 2.83-2.76 (m, 1H), 2.76-2.68 (m, 1H), 1.32 (d, J=6.2 Hz, 3H).

### Example 46

### 5-[(2S,6R)-2-[[2-[2-(1-hydroxyethyl)-4-pyridyl]-2,6-diazaspiro[3.3]heptan-6-yl]methyl]-6-methyl-morpholin-4-yl]quinoline-8-carbonitrile

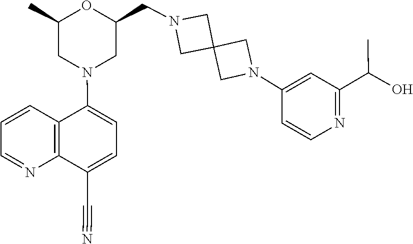 [SMILES: CC(O)c1cc(N2CC3(CN(C[C@H]4CN(c5ccc(C#N)c6ncccc56)C[C@@H](C)O4)C3)C2)ccn1]

The title compound was prepared in analogy to the preparation of Example 1 by using 1-(4-bromo-2-pyridyl)ethanol (CAS: 1471260-48-6, Vendor: Accela ChemBio Inc) instead of (4-bromo-6-methyl-2-pyridyl)methanol (compound 1a). Example 46 (31 mg) was obtained as a light yellow powder. MS: calc'd 485 (MH₊), measured 485 (MH₊). ¹H NMR (400 MHz, METHANOL-d₄) δ=9.01 (dd, J=1.6, 4.3 Hz, 1H), 8.66 (dd, J=1.6, 8.6 Hz, 1H), 8.18 (d, J=7.9 Hz, 1H), 8.02 (d, J=7.2 Hz, 1H), 7.66 (dd, J=4.3, 8.6 Hz, 1H), 7.29 (d, J=8.1 Hz, 1H), 6.65-6.56 (m, 2H), 4.97-4.91 (m, 1H), 4.68-4.39 (m, 8H), 4.29 (br t, J=9.7 Hz, 1H), 4.19-4.09 (m, 1H), 3.53-3.47 (m, 1H), 3.46-3.37 (m, 3H), 2.78 (dd, J=10.5, 11.7 Hz, 1H), 2.71 (dd, J=10.3, 12.2 Hz, 1H), 1.52 (d, J=6.6 Hz, 3H), 1.31 (d, J=6.2 Hz, 3H).

### Example 47

### 5-[2S,6R)-2-[[2-[2-(difluoromethyl)-4-pyridyl]-2,6-diazaspiro[3.3]heptan-6-yl]methyl]-6-methyl-morpholin-4-yl]quinoline-8-carbonitrile

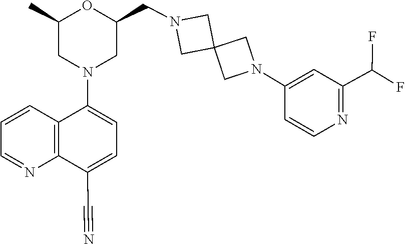 [SMILES: C[C@@H]1CN(c2ccc(C#N)c3ncccc23)C[C@H](CN2CC3(C2)CN(c2ccnc(C(F)F)c2)C3)O1]

The title compound was prepared in analogy to the preparation of Example 1 by using 4-bromo-2-(difluoromethyl)pyridine (CAS: 1211580-54-9, Vendor: BePharm) instead of (4-bromo-6-methyl-2-pyridyl)methanol (compound 1a). Example 47 (5 mg) was obtained as a yellow powder. MS: calc'd 491 (MH₊), measured 491 (MH₊). ¹H NMR (400 MHz, METHANOL-d₄) δ=9.00 (dd, J=1.7, 4.2 Hz, 1H), 8.65 (dd, J=1.7, 8.6 Hz, 1H), 8.17 (dd, J=5.2, 7.4 Hz, 2H), 7.65 (dd, J=4.3, 8.6 Hz, 1H), 7.28 (d, J=8.1 Hz, 1H), 7.07-6.78 (m, 2H), 6.71 (dd, J=2.4, 6.8 Hz, 1H), 4.71-4.41 (m, 8H), 4.27 (br t, J=9.8 Hz, 1H), 4.18-4.09 (m, 1H), 3.53-3.46 (m, 1H), 3.45-3.36 (m, 3H), 2.81-2.73 (m, 1H), 2.69 (dd, J=10.3, 12.0 Hz, 1H), 1.30 (d, J=6.2 Hz, 3H).

### Example 48

### 5-[(2S,6R)-2[[2-[2-(1-hydroxy-1-methyl-ethyl)-4-pyridyl]-2,6-diazaspiro[3.3]heptan-6-yl]methyl]-6-methyl-morpholin-4-yl]quinoline-8-carbonitrile

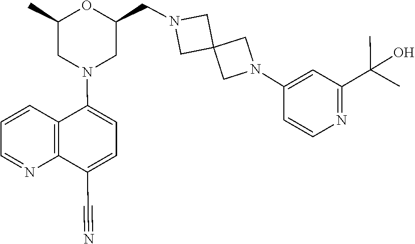 [SMILES: C[C@@H]1CN(c2ccc(C#N)c3ncccc23)C[C@H](CN2CC3(C2)CN(c2ccnc(C(C)(C)O)c2)C3)O1]

The title compound was prepared in analogy to the preparation of Example 1 by using 2-[4-(2,6-diazaspiro[3.3]heptan-2-yl)-2-pyridyl]propan-2-ol (compound 48c) instead of [442,6-diazaspiro[3.3]heptan-2-yl)-6-methyl-2-pyridyl]methanol;2,2,2-trifluoroacetic acid (compound 1d). Example 48 (2 mg) was obtained as a yellow solid. MS: calc'd 499 (MH₊), measured 499

(MH₊). ¹H NMR (400 MHz, METHANOL-d₄) δ=9.01 (dd, J=1.7, 4.2 Hz, 1H), 8.66 (dd, J=1.7, 8.6 Hz, 1H), 8.18 (d, J=7.9 Hz, 1H), 8.00 (d, J=7.2 Hz, 1H), 7.66 (dd, J=4.3, 8.6 Hz, 1H), 7.29 (d, J=8.1 Hz, 1H), 6.67-6.56 (m, 2H), 4.55 (br s, 8H), 4.31-4.22 (m, 1H), 4.18-4.09 (m, 1H), 3.49-3.36 (m, 4H), 2.78 (t, J=11.1 Hz, 1H), 2.70 (dd, J=10.3, 12.0 Hz, 1H), 1.60 (s, 6H), 1.31 (d, J=6.2 Hz, 3H).

The compound 48c was prepared according to the following scheme:

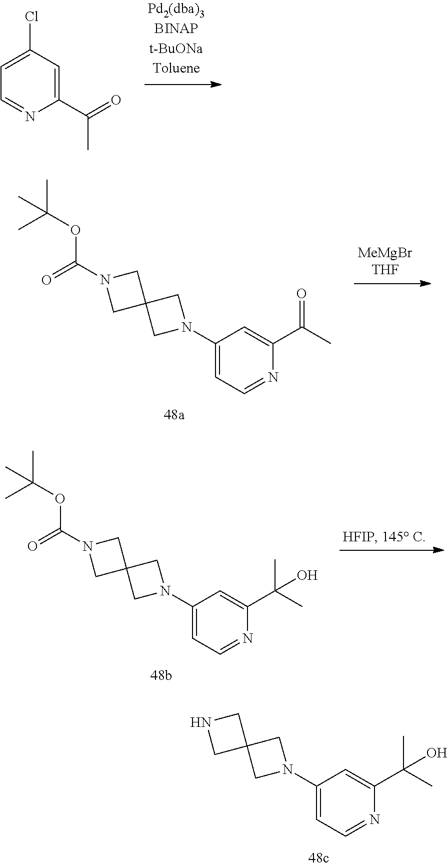

Step 1: preparation of tert-butyl 6-(2-acetyl-4-pyridyl)-2,6-diazaspiro[3.3]heptane-2-carboxylate (compound 48a)

To a microwave tube was added 1-(4-chloro-2-pyridyl)ethanone (CAS: 60159-37-7, Vendor: BePharm, 150 mg, 964 μmol), tert-butyl 2,6-diazaspiro[3.3]heptane-2-carboxylate hemioxalate (328 mg, 675 μmol), sodium tert-butoxide (278 mg, 2.89 mmol) and toluene (4 mL), the suspension was bubbled with N₂ for 5 mins and Pd₂(dba)₃ (88 mg, 96 μmol) and 2,2′-bis(diphenylphosphaneyl)-1,1′-binaphthalene (120 mg, 193 μmol) were added. The tube was sealed and stirred at 100° C. for 12 hrs. After being cooled down, the mixture was diluted with 10 mL EA and filtered through celite. The filtrate was concentrated to give a brown oil which was purified by flash column (EA/PE=0 to 100%) to give compound 48a (85 mg) as a yellow oil. MS: calc'd 318 (MH₊), measured 318 (MH₊).

Step 2: preparation of tert-butyl 6-[2-(1-hydroxy-1-methyl-ethyl)-4-pyridyl]-2,6-diazaspiro[3.3]heptane-2-carboxylate (compound 48b)

To a flask was added tert-butyl 6-(2-acetyl-4-pyridyl)-2,6-diazaspiro[3.3]heptane-2-carboxylate (compound 48a, 85 mg, 268 μmol) and THF (2 mL). After being cooled with dry ice/ethanol bath, the mixture was added with methylmagnesium bromide (3M in Et₂O, 402 μL, 1.21 mmol) portion wise, then warmed to rt slowly and stirred for 2 hrs. Then it was quenched with sat. NH₄Cl and diluted with 20 mL water. The mixture was extracted with EA (20 mL) twice and DCM (15 mL) twice, and the combined organic layer was dried over Na₂SO₄ and concentrated to give compound 48b (70 mg) as an oil. MS: calc'd 334 (MH₊), measured 334 (MH₊).

Step 3: preparation of 2-[4-(2,6-diazaspiro[3.3]heptan-2-yl)-2-pyridyl]propan-2-ol (compound 48c)

The compound 48b was dissolved in 1,1,1,3,3,3-hexafluoro-2-propanol (6.48 g, 4 mL, 38.50 mmol) and heated under microwave at 140° C. for 40 mins. Then the mixture was concentrated directly to give compound 48c (50 mg) as a brown oil. MS: calc'd 234 (MH₊), measured 234 (MH₊).

### Example 49

### 5-[(2S,6R)-2[[2-(2-cyclopropyl-4-pyridyl)-2,6-diazaspiro[3.3]heptan-6-yl]methyl]-6-methyl-morpholin-4-yl]quinoline-8-carbonitrile

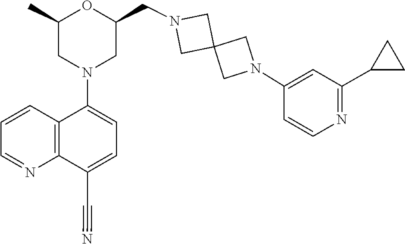 [SMILES: C[C@@H]1CN(c2ccc(C#N)c3ncccc23)C[C@H](CN2CC3(C2)CN(c2ccnc(C4CC4)c2)C3)O1]

The title compound was prepared in analogy to the preparation of Example 1 by using 4-bromo-2-cyclopropylpyridine instead of (4-bromo-6-methyl-2-pyridyl)methanol (compound 1a). Example 49 (25 mg) was obtained as a yellow solid. MS: calc'd 481 (MH₊), measured 481 (MH₊). ¹H NMR (400 MHz, METHANOL-d₄) δ=8.99 (dd, J=1.6, 4.3 Hz, 1H), 8.65 (dd, J=1.6, 8.6 Hz, 1H), 8.16 (d, J=8.1 Hz, 1H), 7.92 (d, J=7.1 Hz, 1H), 7.65 (dd, J=4.3, 8.6 Hz, 1H), 7.27 (d, J=8.1 Hz, 1H), 6.50 (br d, J=4.9 Hz, 1H), 6.26 (s, 1H), 4.64-4.38 (m, 8H), 4.26 (br t, J=9.5 Hz, 1H), 4.17-4.08 (m, 1H), 3.50-3.45 (m, 1H), 3.45-3.42 (m, 1H), 3.41-3.36 (m, 2H), 2.80-2.73 (m, 1H), 2.69 (dd, J=10.4, 12.1 Hz, 1H), 2.13-2.03 (m, 1H), 1.29 (d, J=6.2 Hz, 3H), 1.28-1.23 (m, 2H), 1.08-1.02 (m, 2H).

### Example 50

### 5-[2S,6R)-2-[[4-[2-(hydroxymethyl)-6-methyl-4-pyridyl]piperazin-1-yl]methyl]-6-methyl-morpholin-4-yl]quinoline-8-carbonitrile

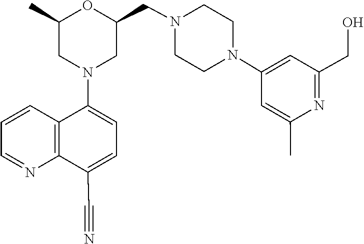 [SMILES: Cc1cc(N2CCN(C[C@H]3CN(c4ccc(C#N)c5ncccc45)C[C@@H](C)O3)CC2)cc(CO)n1]

The title compound was prepared in analogy to the preparation of Example 1 by using tert-butyl piperazine-1-carboxylate instead of tert-butyl 2,6-diazaspiro[3.3]heptane-2-carboxylate;oxalic acid (compound 1b). Example 50 (4 mg) was obtained as a yellow solid. MS: calc'd 473 (MH₊), measured 473 (MH₊). ¹H NMR (400 MHz, METHANOL-d₄) δ=9.01 (dd, J=1.6, 4.3 Hz, 1H), 8.67 (dd, J=1.7, 8.6 Hz, 1H), 8.17 (d, J=8.1 Hz, 1H), 7.66 (dd, J=4.2, 8.6 Hz, 1H), 7.29 (d, J=8.1 Hz, 1H), 7.14 (d, J=2.6 Hz, 1H), 7.08 (d, J=2.4 Hz, 1H), 4.74 (s, 2H), 4.56-4.47 (m, 1H), 4.25-4.16 (m, 1H), 4.07 (br s, 4H), 3.59 (br s, 4H), 3.45 (br d, J=12.8 Hz, 2H), 3.41-3.35 (m, 2H), 2.84-2.70 (m, 2H), 2.60 (s, 3H), 1.33 (d, J=6.2 Hz, 3H).

### Example 51

### 5-[2S,6R)-2-[[8-[2-(hydroxymethyl)-4-pyridyl]-5-oxa-2,8-diazaspiro[3.5]nonan-2-yl]methyl]-6-methyl-morpholin-4-yl]quinoline-8-carbonitrile

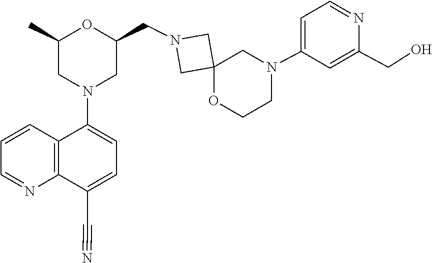 [SMILES: C[C@@H]1CN(c2ccc(C#N)c3ncccc23)C[C@H](CN2CC3(C2)CN(c2ccnc(CO)c2)CCO3)O1]

The title compound was prepared in analogy to the preparation of Example 1 by using (4-bromo-2-pyridyl)methanol and tert-butyl 5-oxa-2,8-diazaspiro[3.5]nonane-2-carboxylate instead of (4-bromo-6-methyl-2-pyridyl)methanol (compound 1a) and tert-butyl 2,6-diazaspiro[3.3]heptane-2-carboxylate;oxalic acid (compound 1b). Example 51 (9 mg) was obtained as a light yellow solid. MS: calc'd 501 (MH₊), measured 501 (MH₊). ¹H NMR (400 MHz, METHANOL-d₄) δ=8.87 (dd, J=1.6, 4.3 Hz, 1H), 8.53 (dd, J=1.6, 8.6 Hz, 1H), 8.05 (d, J=8.1 Hz, 1H), 8.00 (d, J=6.2 Hz, 1H), 7.52 (dd, J=4.3, 8.6 Hz, 1H), 7.14 (d, J=8.1 Hz, 1H), 6.97 (d, J=2.4 Hz, 1H), 6.73 (dd, J=2.6, 6.2 Hz, 1H), 4.50 (s, 2H), 4.01-3.88 (m, 2H), 3.71-3.61 (m, 2H), 3.50 (s, 2H), 3.44 (br dd, J=8.5, 16.7 Hz, 2H), 3.31-3.24 (m, 4H), 3.06 (dd, J=5.7, 8.3 Hz, 2H), 2.70-2.48 (m, 4H), 1.14 (d, J=6.2 Hz, 3H).

### Example 52

The following tests were carried out in order to determine the activity of the compounds of formula (I) and (Ia) in HEK293-Blue-hTLR-7/8/9 cells assay.

HEK293-Blue-hTLR-7 cells assay:

A stable HEK293-Blue-hTLR-7 cell line was purchased from InvivoGen (Cat. #: hkb-htlr7, San Diego, Calif., USA). These cells were originally designed for studying the stimulation of human TLR7 by monitoring the activation of NE-κB. A SEAP (secreted embryonic alkaline phosphatase) reporter gene was placed under the control of the IFN-β minimal promoter fused to five NE-κB and AP-1-binding sites. The SEAP was induced by activating NE-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qbl, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000-450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signalling pathway that TLR7 activation leads to downstream NE-κB activation has been widely accepted, and therefore similar reporter assay was modified for evaluating TLR7 antagonist.

HEK293-Blue-hTLR-8 cells assay:

A stable HEK293-Blue-hTLR-8 cell line was purchased from InvivoGen (Cat. #: hkb-htlr8, San Diego, Calif., USA). These cells were originally designed for studying the stimulation of human TLR8 by monitoring the activation of NE-κB. A SEAP (secreted embryonic alkaline phosphatase) reporter gene was placed under the control of the IFN-β minimal promoter fused to five NE-κB and AP-1-binding sites. The SEAP was induced by activating NE-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat. #: rep-qbl, Invivogen, San Diego, Ca, USA) at a wavelength of 640 nm, a detection medium that turns purple or blue in the presence of alkaline phosphatase.

HEK293-Blue-hTLR8 cells were incubated at a density of 250,000-450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 60 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signalling pathway that TLR8 activation leads to downstream NE-κB activation has been widely accepted, and therefore similar reporter assay was modified for evaluating TLR8 antagonist.

HEK293-Blue-hTLR-9 cells assay:

A stable HEK293-Blue-hTLR-9 cell line was purchased from InvivoGen (Cat. #: hkb-htlr9, San Diego, Calif., USA). These cells were originally designed for studying the stimulation of human TLR9 by monitoring the activation of NE-KB. A SEAP (secreted embryonic alkaline phosphatase) reporter gene was placed under the control of the IFN-β minimal promoter fused to five NE-κB and AP-1-binding sites. The SEAP was induced by activating NE-κB and AP-1 via stimulating HEK-Blue hTLR9 cells with TLR9 ligands. Therefore the reporter expression was declined by TLR9 antagonist under the stimulation of a ligand, such as ODN₂₀₀₆ (Cat. #: tlrl-2006-1, Invivogen, San Diego, Calif., USA), for incubation of 20 hrs. The cell culture supernatant SEAP reporter activity was determined using QUANTI-Blue™ kit (Cat. #: rep-qb1, Invivogen, San Diego, Calif., USA) at a wavelength of 640 nm, a detection medium that turns purple or blue in the presence of alkaline phosphatase.

HEK293-Blue-hTLR9 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM ODN₂₀₀₆ in above DMEM, perform incubation under 37° C. in a CO₂ incubator for 20 hrs. Then 20 μL of the supernatant from each well was incubated with 180 μL Quanti-blue substrate solution at 37° C. for 2 h and the absorbance was read at 620˜655 nm using a spectrophotometer. The signaling pathway that TLR9 activation leads to downstream NE-κB activation has been widely accepted, and therefore similar reporter assay was modified for evaluating TLR9 antagonist.

The compounds of formula (I) have human TLR7 and/or TLR8 inhibitory activities (IC₅₀ value)<0.5 μM. Moreover, some compounds also have human TLR9 inhibitory activity <0.5 μM. Activity data of the compounds of the present invention were shown in Table 2.

TABLE 2
The activity of the compounds of present invention
in HEK293-Blue-hTLR-7/8/9 cells assays

| | HEK/hTLR7 | HEK/hTLR8 | HEK/hTLR9 |
|---|---|---|---|
| Example No | IC₅₀ (μM) | IC₅₀ (μM) | IC₅₀ (μM) |
| R1 (reference | 0.095 | 0.142 | 6.623 |
| compound) | | | |
| R2 (reference | 0.210 | 0.342 | 7.034 |
| compound) | | | |
| 1 | 0.028 | 0.081 | 0.034 |
| 3 | <0.003 | <0.003 | 0.048 |
| 4A | 0.009 | 0.007 | 0.148 |
| 4B | 0.01 | 0.005 | 0.145 |
| 5 | 0.059 | 0.033 | 0.032 |
| 6 | 0.044 | 0.053 | 0.041 |
| 7 | 0.026 | 0.031 | 0.039 |
| 8 | 0.007 | 0.007 | <0.032 |
| 9 | 0.016 | 0.051 | 0.032 |
| 10 | 0.057 | 0.022 | <0.032 |
| 11 | 0.008 | 0.011 | 0.04 |
| 12 | 0.013 | 0.012 | 0.049 |
| 14 | <0.003 | <0.003 | <0.032 |
| 15 | 0.007 | 0.006 | 0.071 |
| 16 | <0.003 | <0.003 | 0.038 |
| 17A | <0.003 | <0.003 | 0.154 |
| 17B | 0.011 | 0.003 | <0.032 |
| 19 | 0.006 | 0.003 | 0.062 |
| 20 | 0.2 | 0.07 | 0.036 |
| 21 | 0.062 | 0.015 | <0.032 |
| 22 | 0.037 | 0.038 | 0.034 |
| 23 | 0.229 | 0.041 | <0.032 |
| 24 | 0.026 | 0.01 | <0.032 |
| 25 | 0.034 | 0.014 | <0.032 |
| 28 | 0.122 | 0.032 | 0.057 |
| 29 | 0.156 | 0.025 | 0.059 |
| 30 | 0.065 | 0.014 | 0.088 |
| 33 | 0.012 | 0.007 | 0.122 |
| 34 | 0.012 | 0.004 | 0.155 |
| 35 | 0.017 | 0.009 | 0.034 |
| 36 | 0.017 | 0.012 | 0.215 |
| 37 | 0.034 | <0.003 | 0.16 |
| 38 | 0.035 | 0.004 | 0.039 |
| 40 | 0.008 | <0.003 | 0.041 |
| 42 | 0.013 | <0.003 | <0.032 |
| 43 | 0.007 | 0.018 | 0.054 |
| 44 | 0.02 | 0.03 | <0.032 |
| 45 | 0.039 | 0.014 | 0.091 |
| 46 | 0.032 | 0.09 | <0.032 |
| 48 | 0.055 | 0.198 | 0.148 |
| 49 | 0.013 | 0.02 | 0.034 |
| 50 | 0.01 | 0.004 | 0.134 |
| 51 | <0.003 | 0.003 | 0.035 |

### Example 53

hERG channel inhibition assay:

The hERG channel inhibition assay is a highly sensitive measurement that identifies compounds exhibiting hERG inhibition related to cardiotoxicity in vivo. The hERG K⁺ channels were cloned in humans and stably expressed in a CHO (Chinese hamster ovary) cell line. CHO_{hERG} cells were used for patch-clamp (voltage-clamp, whole-cell) experiments. Cells were stimulated by a voltage pattern to activate hERG channels and conduct I_{KhERG} currents (rapid delayed outward rectifier potassium current of the hERG channel). After the cells were stabilized for a few minutes, the amplitude and kinetics of I_{KhERG} were recorded at a stimulation frequency of 0.1 Hz (6 bpm). Thereafter, the test compound was added to the preparation at increasing concentrations. For each concentration, an attempt was made to reach a steady-state effect, usually, this was achieved within 3-10 min at which time the next highest concentration was applied. The amplitude and kinetics of I_{KhERG} are recorded in each concentration of the drug which were compared to the control values (taken as 100%). (references: Redfern W S, Carlsson L, Davis A S, Lynch W G, MacKenzie I, Palethorpe S, Siegl P K, Strang I, Sullivan A T, Wallis R, Camm A J, Hammond T G. 2003; Relationships between preclinical cardiac electrophysiology, clinical QT interval prolongation and torsade de pointes for a broad range of drugs: evidence for a provisional safety margin in drug development. Cardiovasc. Res. 58:32-45, Sanguinetti M C, Tristani-Firouzi M. 2006; hERG potassium channels and cardiac arrhythmia. Nature 440:463-469, Webster R, Leishman D, Walker D. 2002; Towards a drug concentration effect relationship for QT prolongation and torsades de pointes. Curr. Opin. Drug Discov. Devel. 5:116-26).

Results of hERG are given in Table 3. A safety ratio (hERG IC₂₀/EC₅₀) >30 suggests a sufficient window to differentiate the pharmacology by inhibiting TLR7/8/9 pathways from the potential hERG related cardiotoxicity. According to the calculation of hERG IC₂₀/TLR7/8/9 IC₅₀ below which serves as early selectivity index to assess hERG liability. obviously reference compounds ER-887258, ER-888285, ER-888286,R1 and R2 have much narrower safety window compared to the compounds of this invention.

TABLE 3
hERG and safety ratio results

| | | | hERG | hERG | hERG |
|---|---|---|---|---|---|
| | hERG | hERG | IC₂₀/ | IC₂₀/ | IC₂₀/ |
| Example | IC₂₀ | IC₅₀ | TLR7 | TLR8 | TLR9 |
| No | (μM) | (μM) | IC₅₀ | IC₅₀ | IC₅₀ |
| ER-887258 | 0.687 | 2.784 | 8.1 | N.A. | 0.3 |
| ER-888285 | 1.006 | 3.105 | 8.4 | N.A. | 0.5 |
| ER-888286 | 0.348 | 1.297 | 0.3 | N.A. | 0.2 |
| R1 | 0.879 | 2.745 | 9.3 | 6.2 | 0.1 |
| R2 | 0.280 | 0.770 | 1.3 | 0.8 | 0.0 |
| 1 | >10.000 | >20.000 | >357.1 | >123.5 | >294.1 |
| 5 | 7.184 | >20.000 | 121.8 | 217.7 | 224.5 |
| 6 | 5.825 | >20.000 | 132.4 | 109.9 | 142.1 |
| 7 | >10.000 | >20.000 | >384.6 | >322.6 | >256.4 |
| 30 | 4.508 | >10.000 | 69.4 | 322.0 | 51.2 |
| 45 | >10.000 | >20.000 | >256.4 | >714.3 | >109.9 |
| 46 | 4.690 | >10.000 | 146.6 | 52.1 | 146.6 |

### Example 54

The compounds would be desirable to have minimal DDI liabilities. Therefore, the effects of compounds of formula (I) or (Ia) on CYP2D6 are determined.

CYP inhibition assay

This is a high throughput screening assay used for assessment of reversible inhibition of CYP2D6 activity of test compounds in human liver microsome (HLM) in early discovery stage.

TABLE 4
Chemicals and materials used in the CYP inhibition assay

| | | | | Final | |
|---|---|---|---|---|---|
| | | | | Concentration | |
| Substances | Description | Source | Cat. No. | in incubation | |
| Human Liver | | BD-Gentest | 452117 | 0.2 | mg/mL |
| Microsomes | | | | | |
| Dextromethorphan | CYP2D6 substrate | Sigma | D-2531 | 5 | μM |
| Dextrorphan | CYP2D6 product | | | | |
| Dextrorphan-D3 | CYP2D6 internal | Promochem | CERD-041 | | |
| | standard | | | | |
| Quinidine | CYP2D6 inhibitor | | | 0.5 | μM |

### Procedure

10 mM DMSO stock solutions of test compounds were diluted in DMSO to generate 2 mM intermediate stock solution. 250 nL of intermediate stock solution were transferred in duplicate into 3 separate 384 well microtitre plates (assay-ready plates). A mixture of HLM and each substrate was made up. 45 μL of HLM substrate mix was then transferred to each well of an assay ready plate and mixed. The negative (solvent) and positive control (standard inhibitor for CYP 2D6) were included in each assay ready plate. The assay ready plate was warmed to 37° C. in an incubator over 10 minutes. 5 μL pre-warmed NADPH regenerating system was added to each incubation well to start the reaction. Final incubation volume was 50 μL. The assay plate then was placed back in the 37° C. incubator. After 10 minutes incubation, incubates were quenched by addition of 50 μL 100% acetonitrile containing internal standards (20 ng/mL D3-Dextrorphan). The supernatants were collected for RapidFire/MS/MS analysis.

RapidFire online solid phase extraction/sample injection system (Agilent) coupled with API4000 triple quadrupole mass spectrometer (AB Sciex) were used for sample analysis. The mobile phase composed of acetonitrile and water supplemented with 0.1% formic acid. A C4 solid phase extraction cartridge is used for sample separation. MS detection is achieved in positive ion MRM mode.

Data analysis Peak areas for substrate, metabolite and internal standard are determined using the RapidFire integrator software (version 3.6.12009.12296). Peak area ratios (PAR) of metabolite and internal standard (stable-labelled metabolite) are then calculated. The measurement window for each experiment is then defined:

PAR (0% activity), average PAR for all incubations containing concentrated inhibitor; Par (100% activity), average PAR for all incubations containing no inhibitor (DMSO controls);

% Activity (test inhibitor)=

=[PAR(test inhibitor)-PAR(0% activity)]/[PAR(100% activity)-PAR(0% activity)];

% Inhibition (test inhibitor)=100-% Activity (test inhibitor).

The compounds of present invention were found to have low CYP inhibition for CYP2D6 determined in the assays described above.

TABLE 5
CYP inhibition for CYP2D6

| | CYP 2D6 inhibition |
|---|---|
| Example No | %@10 μM |
| ER-888286 | 52.5 |
| 1 | 6 |
| 3 | 6 |
| 5 | 7.5 |
| 6 | 8 |
| 7 | −21.5 |
| 9 | −5 |
| 10 | 3.5 |
| 11 | −7.5 |
| 12 | −3 |
| 14 | 19 |
| 15 | 4 |
| 16 | 5 |
| 17B | 5 |
| 21 | 2 |
| 22 | 2 |
| 24 | −9.5 |
| 25 | 8.5 |
| 30 | 8.5 |
| 35 | −4 |
| 38 | 0 |
| 39 | 7.5 |
| 40 | 14.5 |
| 42 | −0.5 |
| 43 | −13 |
| 44 | 5 |
| 45 | −2.5 |
| 46 | 17 |
| 50 | 10.5 |
| 51 | −15.5 |

ND: not detected; percentage inhibition <0: not or weak inhibitor

### Example 55

Human microsome stability assay

The human microsomal stability assay is used for early assessment of metabolic stability of a test compound in human liver microsomes.

Human liver microsomes (Cat.NO.: 452117, Corning, USA;Cat.NO.:H2610, Xenotech, USA) were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 6 below.

TABLE 6
Human liver microsome stability of
the compounds of present invention

| | Clearance of Human |
|---|---|
| Example No | microsome (mL/min/kg) |
| ER-887258 | 17.86 |
| ER-888285 | 17.15 |
| R1 | 10.84 |
| R2 | 15.43 |
| 1 | 6.35 |
| 4A | 6.15 |
| 5 | 7.47 |
| 6 | 6.15 |
| 7 | 6.15 |
| 9 | 6.15 |
| 20 | 6.15 |
| 26 | 7.72 |
| 27 | 7.67 |
| 28 | 6.25 |
| 30 | 7.14 |
| 31 | 7.61 |
| 37 | 6.15 |
| 40 | 6.15 |
| 44 | 7.21 |
| 46 | 7.66 |
| 48 | 6.87 |
| 49 | 6.39 |
| 50 | 7.72 |

1. A compound of formula (I), 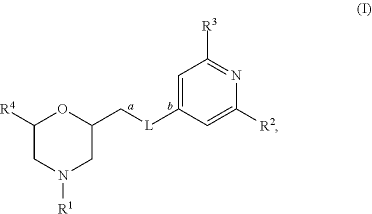
wherein
R¹ is 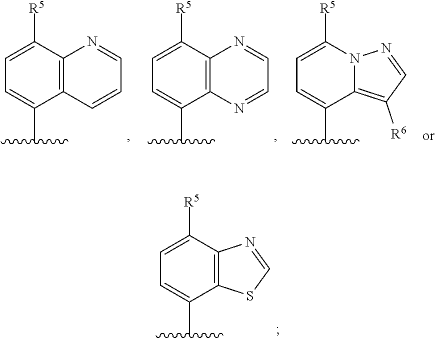
wherein R⁵ is cyano or halogen; R⁶ is H or halogen;
R² is H, amino or C₁₋₆alkyl;
R³ is amino, C₁₋₆alkylamino, C₁₋₆alkyl, haloC₁₋₆alkyl, heterocyclyl, hydroxyC₁₋₆alkyl or C₃₋₇cycloalkyl;
R⁴ is C₁₋₆alkyl;
L is 1,3,3a,4,6,6a-hexahydropyrrolo[3,4-c]pyrrolyl; 1,6-diazaspiro[3.3]heptanyl; 2,3,4a,5,7,7a-hexahydropyrrolo[3,4-b][1,4]oxazinyl; 2,6-diazaspiro[3.3]heptanyl; 2,7-diazaspiro[3.4]octanyl; 5-oxa-2,8-diazaspiro[3.5]nonanyl; (C₁₋₆alkyl)aminoazetidinyl; aminoazetidinyl; azetidinyl(C₁₋₆alkyl)amino; azetidinylamino; (phenylC₁₋₆alkyl)piperazinyl; (hydroxyC₁₋₆alkyl)piperazinyl; (C₁₋₆alkyl)piperazinyl; piperazinyl; piperidinyl; (C₁₋₆alkyl)aminopiperidinyl; aminohalopiperidinyl; amino(hydroxy)piperidinyl; aminopiperidinyl; piperidinylamino; amino(hydroxy)pyrrolidinyl; aminopyrrolidinyl; or pyrrolidinylamino;
or a pharmaceutically acceptable salt, enantiomer or diastereomer thereof.

2. A compound of formula (Ia), 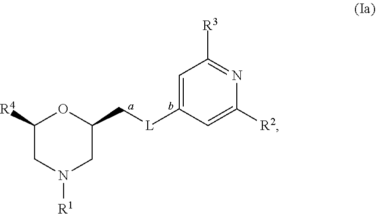
wherein
R¹ is 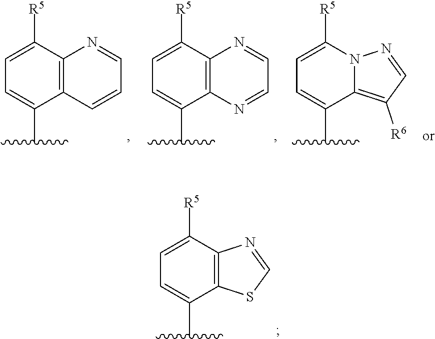
wherein R⁵ is cyano or halogen; R⁶ is H or halogen;
R² is H, amino or C₁₋₆alkyl;
R³ is amino, C₁₋₆alkylamino, C₁₋₆alkyl, haloC₁₋₆alkyl, heterocyclyl, hydroxyC₁₋₆alkyl or C₃₋₇cycloalkyl;
R⁴ is C₁₋₆alkyl;
L is 1,3,3a,4,6,6a-hexahydropyrrolo[3,4-c]pyrrolyl; 1,6-diazaspiro[3.3]heptanyl; 2,3,4a,5,7,7a-hexahydropyrrolo[3,4-b][1,4]oxazinyl; 2,6-diazaspiro[3.3]heptanyl; 2,7-diazaspiro[3.4]octanyl; 5-oxa-2,8-diazaspiro[3.5]nonanyl; (C₁₋₆alkyl)aminoazetidinyl; aminoazetidinyl; azetidinyl(C₁₋₆alkyl)amino; azetidinylamino; (phenylC₁₋₆alkyl)piperazinyl; (hydroxyC₁₋₆alkyl)piperazinyl; (C₁₋₆alkyl)piperazinyl; piperazinyl; piperidinyl; (C₁₋₆alkyl)aminopiperidinyl; aminohalopiperidinyl; amino(hydroxy)piperidinyl; aminopiperidinyl; piperidinylamino; amino(hydroxy)pyrrolidinyl; aminopyrrolidinyl; or pyrrolidinylamino;
or a pharmaceutically acceptable salt, enantiomer or diastereomer thereof.

3. A compound according to claim 1 or 2, wherein R¹ is 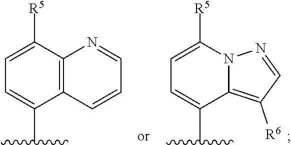
wherein R⁵ is cyano, R⁶ is H.

4. A compound according to claim 3, wherein L is 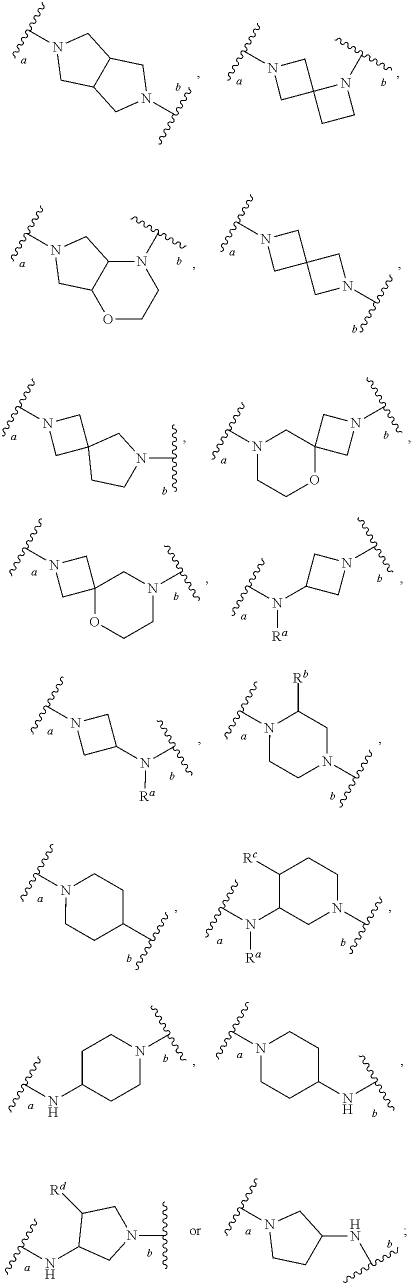
wherein R^{a} is H or C₁₋₆alkyl; R^{b} is H, phenylC₁₋₆alkyl, hydroxyC₁₋₆alkyl or C₁₋₆alkyl; R^{c} is H, halogen or hydroxy; R^{d} is H or hydroxy.

5. A compound according to claim 4, wherein L is 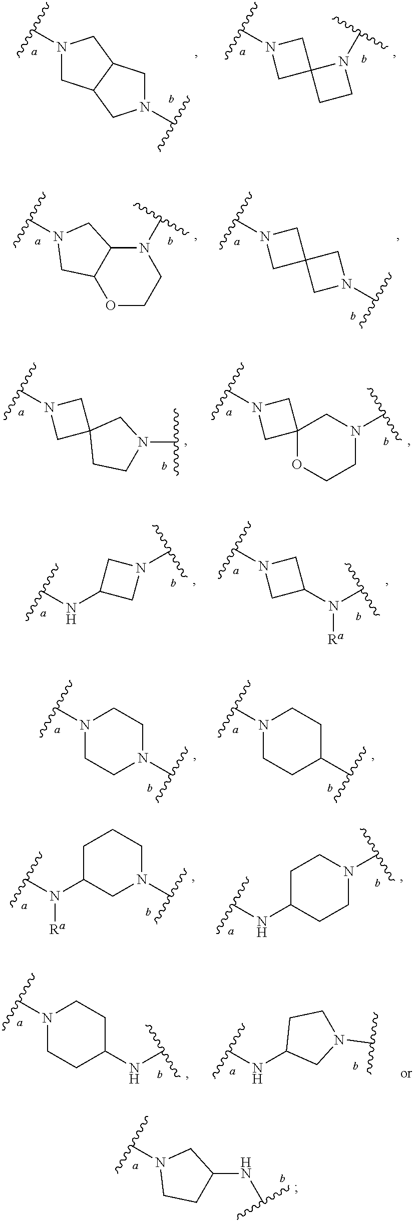
wherein R^{a} is H or C₁₋₆alkyl.

6. A compound according to claim 5, wherein L is

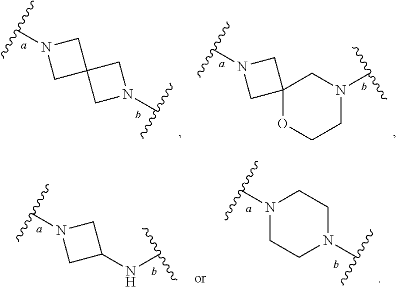 [SMILES: *N1CC2(C1)CN(*)C2 ; *N1CCOC2(C1)CN(*)C2 ; *NC1CN(*)C1 ; *N1CCN(*)CC1]

7. A compound according to claim 6, wherein R³ is amino, C₁₋₆alkylamino, C₁₋₆alkyl, hydroxyC₁₋₆alkyl or C₃₋₇cycloalkyl.

8. A compound according to claim 7, wherein R³ is amino, cyclopropyl, hydroxyethyl, hydroxymethyl, methyl or methylamino.

9. A compound according to claim 1 or 2, wherein
R¹ is 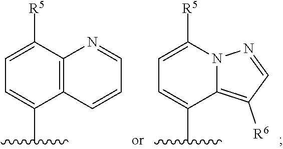
wherein R⁵ is cyano; R⁶ is H;
R² is H or C₁₋₆alkyl;
R³ is C₁₋₆alkyl, hydroxyC₁₋₆alkyl or C₃₋₇cycloalkyl;
R⁴ is C₁₋₆alkyl;
L is 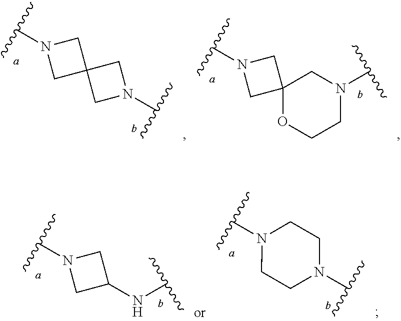 [SMILES: *N1CC2(C1)CN(*)C2 ; *N1CCOC2(C1)CN(*)C2 ; *NC1CN(*)C1 ; *N1CCN(*)CC1]
or a pharmaceutically acceptable salt, enantiomer or diastereomer thereof.

10. A compound according to claim 9, wherein
R¹ is 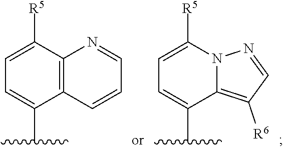
wherein R⁵ is cyano; R⁶ is H;
R² is H or methyl;
R³ is methyl, hydroxymethyl or cyclopropyl;
R⁴ is methyl;
L is 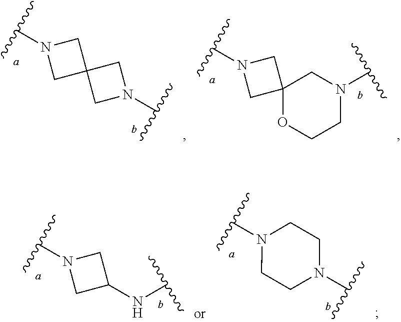 [SMILES: *N1CC2(C1)CN(*)C2 ; *N1CCOC2(C1)CN(*)C2 ; *NC1CN(*)C1 ; *N1CCN(*)CC1]
or a pharmaceutically acceptable salt, enantiomer or diastereomer thereof.

11. A compound selected from:
5-[(2S,6R)-2-[[2-[2-(hydroxymethyl)-6-methyl-4-pyridyl]-2,6-diazaspiro[3.3]heptan-6-yl]methyl]-6-methyl-morpholin-4-yl]quinoline-8-carbonitrile;
5-[(2S,6R)-2-[[8-(2-cyclopropyl-4-pyridyl)-5-oxa-2,8-diazaspiro[3.5]nonan-2-yl]methyl]-6-methyl-morpholin-4-yl]quinoline-8-carbonitrile;
5-[(2S,6R)-2-[[4-[2-(hydroxymethyl)-6-methyl-4-pyridyl]-2-methyl-piperazin-1-yl]methyl]-6-methyl-morpholin-4-yl]quinoline-8-carbonitrile;
5-[(2S,6R)-2-[[(2S)-4-[2-(hydroxymethyl)-6-methyl-4-pyridyl]-2-methyl-piperazin-1-yl]methyl]-6-methyl-morpholin-4-yl]quinoline-8-carbonitrile;
5-[(2S,6R)-2-[[(2R)-4-[2-(hydroxymethyl)-6-methyl-4-pyridyl]-2-methyl-piperazin-1-yl]methyl]-6-methyl-morpholin-4-yl]quinoline-8-carbonitrile;
5-[(2S,6R)-2-[[4-[[2-(hydroxymethyl)-6-methyl-4-pyridyl]amino]-1-piperidyl]methyl]-6-methyl-morpholin-4-yl]quinoline-8-carbonitrile;
5-[(2S,6R)-2-[[3-[[2-(hydroxymethyl)-6-methyl-4-pyridyl]amino]pyrrolidin-1-yl]methyl]-6-methyl-morpholin-4-yl]quinoline-8-carbonitrile;
5-[(2S,6R)-2-[[3-[(2,6-dimethyl-4-pyridyl)amino]azetidin-1-yl]methyl]-6-methyl-morpholin-4-yl]quinoline-8-carbonitrile;
5-[(2S,6R)-2-[[1-(2,6-dimethyl-4-pyridyl)-1,6-diazaspiro[3.3]heptan-6-yl]methyl]-6-methyl-morpholin-4-yl]quinoline-8-carbonitrile;
5-[(2S,6R)-2-[[7-(2,6-dimethyl-4-pyridyl)-2,7-diazaspiro[3.4]octan-2-yl]methyl]-6-methyl-morpholin-4-yl]quinoline-8-carbonitrile;
cis-5-[(2S,6R)-2-[[5-[2-(hydroxymethyl)-6-methyl-4-pyridyl]-1,3,3a,4,6,6a-hexahydropyrrolo[3,4-c]pyrrol-2-yl]methyl]-6-methyl-morpholin-4-yl]quinoline-8-carbonitrile;
5-[(2S,6R)-2-[[3-[(2,6-dimethyl-4-pyridyl)-methyl-amino]azetidin-1-yl]methyl]-6-methyl-morpholin-4-yl]quinoline-8-carbonitrile;
5-[(2S,6R)-2-[[1-[2-(hydroxymethyl)-6-methyl-4-pyridyl]-1,6-diazaspiro[3.3]heptan-6-yl]methyl]-6-methyl-morpholin-4-yl]quinoline-8-carbonitrile;
5-[(2S,6R)-2-[[2-(2,6-dimethyl-4-pyridyl)-5-oxa-2,8-diazaspiro[3.5]nonan-8-yl]methyl]-6-methyl-morpholin-4-yl]quinoline-8-carbonitrile;
5-[(2S,6R)-2-[[8-(2,6-dimethyl-4-pyridyl)-5-oxa-2,8-diazaspiro[3.5]nonan-2-yl]methyl]-6-methyl-morpholin-4-yl]quinoline-8-carbonitrile;
5-[(2R,6S)-2-methyl-6-[[1-(2-methyl-4-pyridyl)-1,6-diazaspiro[3.3]heptan-6-yl]methyl]morpholin-4-yl]quinoline-8-carbonitrile;
5-[(2R,6S)-2-methyl-6-[[8-(2-methyl-4-pyridyl)-5-oxa-2,8-diazaspiro[3.5]nonan-2-yl]methyl]morpholin-4-yl]quinoline-8-carbonitrile;
5-[(2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile;
trans-5-[(2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile;
cis-5-[(2S,6R)-2-[[4-(2,6-dimethyl-4-pyridyl)-2,3,4a,5,7,7a-hexahydropyrrolo[3,4-b][1,4]oxazin-6-yl]methyl]-6-methyl-morpholin-4-yl]quinoline-8-carbonitrile;
5-[(2S,6R)-2-[[4-(2-amino-4-pyridyl)piperazin-1-yl]methyl]-6-methyl-morpholin-4-yl]quinoline-8-carbonitrile;
5-[(2S,6R)-2-[[[1-(2-amino-4-pyridyl)-4-piperidyl]amino]methyl]-6-methyl-morpholin-4-yl]quinoline-8-carbonitrile;
5-[(2S,6R)-2-[[[(3R)-1-(2-amino-4-pyridyl)-3-piperidyl]amino]methyl]-6-methyl-morpholin-4-yl]quinoline-8-carbonitrile;
5-[(2S,6R)-2-[[[(3S)-1-(2-amino-4-pyridyl)-3-piperidyl]amino]methyl]-6-methyl-morpholin-4-yl]quinoline-8-carbonitrile;
5-[(2S,6R)-2-[[[1-[2-(hydroxymethyl)-6-methyl-4-pyridyl]-4-piperidyl]amino]methyl]-6-methyl-morpholin-4-yl]quinoline-8-carbonitrile;
5-[(2S,6R)-2-[[[(3R)-1-[2-(hydroxymethyl)-6-methyl-4-pyridyl]-3-piperidyl]amino]methyl]-6-methyl-morpholin-4-yl]quinoline-8-carbonitrile;
5-[(2S,6R)-2-[[[(3S)-1-[2-(hydroxymethyl)-6-methyl-4-pyridyl]-3-piperidyl]amino]methyl]-6-methyl-morpholin-4-yl]quinoline-8-carbonitrile;
trans-5-[(2S,6R)-2-[[[3-hydroxy-1-[2-(hydroxymethyl)-6-methyl-4-pyridyl]-4-piperidyl]amino]methyl]-6-methyl-morpholin-4-yl]quinoline-8-carbonitrile;
trans-5-[(2S,6R)-2-[[[4-hydroxy-1-[2-(hydroxymethyl)-6-methyl-4-pyridyl]-3-piperidyl]amino]methyl]-6-methyl-morpholin-4-yl]quinoline-8-carbonitrile;
5-[(2S,6R)-2-[[[(3R)-1-[2-(hydroxymethyl)-6-methyl-4-pyridyl]pyrrolidin-3-yl]amino]methyl]-6-methyl-morpholin-4-yl]quinoline-8-carbonitrile;
5-[(2S,6R)-2-[[[(3S)-1-[2-(hydroxymethyl)-6-methyl-4-pyridyl]pyrrolidin-3-yl]amino]methyl]-6-methyl-morpholin-4-yl]quinoline-8-carbonitrile;
5-[(2S,6R)-2-[[[1-[2-(hydroxymethyl)-6-methyl-4-pyridyl]azetidin-3-yl]amino]methyl]-6-methyl-morpholin-4-yl]quinoline-8-carbonitrile;
trans-5-[(2S,6R)-2-[[[4-hydroxy-1-[2-(hydroxymethyl)-6-methyl-4-pyridyl]pyrrolidin-3-yl]amino]methyl]-6-methyl-morpholin-4-yl]quinoline-8-carbonitrile;
trans-5-[(2S,6R)-2-[[[1-(2,6-dimethyl-4-pyridyl)-4-hydroxy-3-piperidyl]amino]methyl]-6-methyl-morpholin-4-yl]quinoline-8-carbonitrile;
cis-5-[(2S,6R)-2-[[[4-fluoro-1-[2-(hydroxymethyl)-6-methyl-4-pyridyl]-3-piperidyl]amino]methyl]-6-methyl-morpholin-4-yl]quinoline-8-carbonitrile;
trans-5-[(2S,6R)-2-[[[4-fluoro-1-[2-(hydroxymethyl)-6-methyl-4-pyridyl]-3-piperidyl]amino]methyl]-6-methyl-morpholin-4-yl]quinoline-8-carbonitrile;
5-[(2S,6R)-2-[[[(3R)-1-[2-(hydroxymethyl)-6-methyl-4-pyridyl]-3-piperidyl]-methyl-amino]methyl]-6-methyl-morpholin-4-yl]quinoline-8-carbonitrile;
5-[(2S,6R)-2-[[[5,5-difluoro-1-[2-(hydroxymethyl)-6-methyl-4-pyridyl]-3-piperidyl]amino]methyl]-6-methyl-morpholin-4-yl]quinoline-8-carbonitrile;
5-[(2S,6R)-2-[[[1-[2-(hydroxymethyl)-6-methyl-4-pyridyl]azetidin-3-yl]-methyl-amino]methyl]-6-methyl-morpholin-4-yl]quinoline-8-carbonitrile;
5-[(2S,6R)-2-[[[(3S)-1-[2-(hydroxymethyl)-6-methyl-4-pyridyl]-3-piperidyl]-methyl-amino]methyl]-6-methyl-morpholin-4-yl]quinoline-8-carbonitrile;
4-[(2S,6R)-2-[[2-benzyl-4-(2,6-dimethyl-4-pyridyl)piperazin-1-yl]methyl]-6-methyl-morpholin-4-yl]pyrazolo[1,5-a]pyridine-7-carbonitrile;
5-[(2S,6R)-2-[[4-(2-amino-6-methyl-4-pyridyl)piperazin-1-yl]methyl]-6-methyl-morpholin-4-yl]quinoline-8-carbonitrile;
5-[(2S,6R)-2-[[(2R)-4-(2-amino-6-methyl-4-pyridyl)-2-(hydroxymethyl)piperazin-1-yl]methyl]-6-methyl-morpholin-4-yl]quinoline-8-carbonitrile;
5-[(2R,6S)-2-methyl-6-[[4-[2-methyl-6-(methylamino)-4-pyridyl]piperazin-1-yl]methyl]morpholin-4-yl]quinoline-8-carbonitrile;
5-[(2S,6R)-2-[[4-(2-amino-6-methyl-4-pyridyl)-1-piperidyl]methyl]-6-methyl-morpholin-4-yl]quinoline-8-carbonitrile;
5-[(2S,6R)-2-[[2-[2-(1-hydroxyethyl)-6-methyl-4-pyridyl]-2,6-diazaspiro[3.3]heptan-6-yl]methyl]-6-methyl-morpholin-4-yl]quinoline-8-carbonitrile;
5-[(2S,6R)-2-[[4-[2-amino-6-(hydroxymethyl)-4-pyridyl]piperazin-1-yl]methyl]-6-methyl-morpholin-4-yl]quinoline-8-carbonitrile;
5-[(2S,6R)-2-[[2-[2-(1-hydroxyethyl)-4-pyridyl]-2,6-diazaspiro[3.3]heptan-6-yl]methyl]-6-methyl-morpholin-4-yl]quinoline-8-carbonitrile;
5-[(2S,6R)-2-[[2-[2-(difluoromethyl)-4-pyridyl]-2,6-diazaspiro[3.3]heptan-6-yl]methyl]-6-methyl-morpholin-4-yl]quinoline-8-carbonitrile;
5-[(2S,6R)-2-[[2-[2-(1-hydroxy-1-methyl-ethyl)-4-pyridyl]-2,6-diazaspiro[3.3]heptan-6-yl]methyl]-6-methyl-morpholin-4-yl]quinoline-8-carbonitrile;
5-[(2S,6R)-2-[[2-(2-cyclopropyl-4-pyridyl)-2,6-diazaspiro[3.3]heptan-6-yl]methyl]-6-methyl-morpholin-4-yl]quinoline-8-carbonitrile;
5-[(2S,6R)-2-[[4-[2-(hydroxymethyl)-6-methyl-4-pyridyl]piperazin-1-yl]methyl]-6-methyl-morpholin-4-yl]quinoline-8-carbonitrile; and
5-[(2S,6R)-2-[[8-[2-(hydroxymethyl)-4-pyridyl]-5-oxa-2,8-diazaspiro[3.5]nonan-2-yl]methyl]-6-methyl-morpholin-4-yl]quinoline-8-carbonitrile;
or a pharmaceutically acceptable salt, enantiomer or diastereomer thereof.

12. A process for the preparation of a compound according to any one of claims 1 to 11 comprising any of the following step:
a) the coupling of compound of formula (IX), 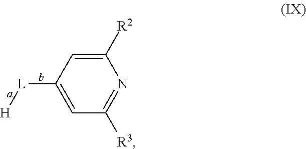
with compound of formula (IV) in the presence of a base;
wherein the base in step a) and b) is K₂CO₃, DIPEA, or Cs₂CO₃; R², R³ and L are defined as in any one of claims 1 to 10.

13. A compound or pharmaceutically acceptable salt, enantiomer or diastereomer according to any one of claims 1 to 11 for use as therapeutically active substance.

14. A pharmaceutical composition comprising a compound in accordance with any one of claims 1 to 11 and a therapeutically inert carrier.

15. The use of a compound according to any one of claims 1 to 11 for the treatment or prophylaxis of systemic lupus erythematosus or lupus nephritis.

16. The use of a compound according to any one of claims 1 to 11 for the preparation of a medicament for the treatment or prophylaxis of systemic lupus erythematosus or lupus nephritis.

17. The use of a compound according to any one of claims 1 to 11 as the TLR7 or TLR8 or TLR9 antagonist.

18. The use of a compound according to any one of claims 1 to 11 as the TLR7 and TLR8 antagonist.

19. The use of a compound according to any one of claims 1 to 11 for the preparation of a medicament for TLR7 and TLR8 and TLR9 antagonist.

20. A compound or pharmaceutically acceptable salt, enantiomer or diastereomer according to any one of claims 1 to 11 for the treatment or prophylaxis of systemic lupus erythematosus or lupus nephritis.

21. A compound or pharmaceutically acceptable salt, enantiomer or diastereomer according to any one of claims 1 to 11, when manufactured according to a process of claim 12.

22. A method for the treatment or prophylaxis of systemic lupus erythematosus or lupus nephritis, which method comprises administering a therapeutically effective amount of a compound as defined in any one of claims 1 to 11.
